SEQUENCE LISTING (13 sequences)

SEQ ID NO: 1 - DNA, 1788 bp, Homo sapiens
gagaccaaaggctaccacagtctccctgaaggtctagatatggaaagacggtggggtcaagtttctcaggctgtggagcgttcttccctgggacctacagagaggaccgatgagaataactacatggagattgtcaacgtaagctgtgtttccggtgctattccaaacaacagtactcaaggaagcagcaaagaaaaacaagaactactcccttgccttcagcaagacaataatcggcctgggattttaacatctgatattaaaactgagctggaatctaaggaactttcagcaactgtagctgagtccatgggtttatatatggattctgtaagagatgctgactattcctatgagcagcagaaccaacaaggaagcatgagtccagctaagatttatcagaatgttgaacagctggtgaaattttacaaaggaaatggccatcgtccttccactctaagttgtgtgaacacgcccttgagatcatttatgtctgactctgggagctccgtgaatggtggcgtcatgcgcgccattgttaaaagccctatcatgtgtcatgagaaaagcccgtctgtttgcagccctctgaacatgacatcttcggtttgcagccctgctggaatcaactctgtgtcctccaccacagccagctttggcagttttccagtgcacagcccaatcacccagggaactcctctgacatgctcccctaatgctgaaaatcgaggctccaggtcgcacagccctgcacatgctagcaatgtgggctctcctctctcaagtccgttaagtagcatgaaatcctcaatttccagccctccaagtcactgcagtgtaaaatctccagtctccagtcccaataatgtcactctgagatcctctgtgtctagccctgcaaatattaacaactcaaggtgctctgtttccagcccttcgaacactaataacagatccacgctttccagtccggcagccagtactgtgggatctatctgtagccctgtaaacaatgccttcagctacactgcttctggcacctctgctggatccagtacattgcgggatgtggttcccagtccagacacgcaggagaaaggtgctcaagaggtcccttttcctaagactgaggaagtagagagtgccatctcaaatggtgtgactggccagcttaatattgtccagtacataaaaccagaaccagatggagcttttagcagctcatgtctaggaggaaatagcaaaataaattcggattcttcattctcagtaccaataaagcaagaatcaaccaagcattcatgttcaggcacctcttttaaagggaatccaacagtaaacccgtttccatttatggatggctcgtatttttcctttatggatgataaagactattattccctatcaggaattttaggaccacctgtgcccggctttgatggtaactgtgaaggcagcggattcccagtgggtattaaacaagaaccagatgacgggagctattacccagaggccagcatcccttcctctgctattgttggggtgaattcaggtggacagtccttccactacaggattggtgctcaaggtacaatatctttatcacgatcggctagagaccaatctttccaacacctgagttcctttcctcctgtcaatactttagtggagtcatggaaatcacacggcgacctgtcgtctagaagaagtgatgggtatccggtcttagaatacattccagaaaatgtatcaagctctactttacgaagtgtttctactggatcttca

SEQ ID NO: 2 - protein, 597 aa, Homo sapiens
METKGYHSLPEGLDMERRWGQVSQAVERSSLGPTERTDENNYMEIVNVSCVSGAIPNNSTQGSSKEKQELLPCLQQDNNRPGILTSDIKTELESKELSATVAESMGLYMDSVRDADYSYEQQNQQGSMSPAKIYQNVEQLVKFYKGNGHRPSTLSCVNTPLRSFMSDSGSSVNGGVMRAIVKSPIMCHEKSPSVCSPLNMTSSVCSPAGINSVSSTTASFGSFPVHSPITQGTPLTCSPNAENRGSRSHSPAHASNVGSPLSSPLSSMKSSISSPPSHCSVKSPVSSPNNVTLRSSVSSPANINNSRCSVSSPSNTNNRSTLSSPAASTVGSICSPVNNAFSYTASGTSAGSSTLRDVVPSPDTQEKGAQEVPFPKTEEVESAISNGVTGQLNIVQYIKPEPDGAFSSSCLGGNSKINSDSSFSVPIKQESTKHSCSGTSFKGNPTVNPFPFMDGSYFSFMDDKDYYSLSGILGPPVPGFDGNCEGSGFPVGIKQEPDDGSYYPEASIPSSAIVGVNSGGQSFHYRIGAQGTISLSRSARDQSFQHLSSFPPVNTLVESWKSHGDLSSRRSDGYPVLEYIPENVSSSTLRSVSTGSS

SEQ ID NO: 3 - DNA, 135 bp, Homo sapiens
atgttgggggacggggattccccggggccgggatttaccccccacgactccgccccctacggcgctctggatatggccgacttcgagtttgagcagatgtttaccgatgcccttggaattgacgagtacggtggg

SEQ ID NO: 4 - DNA, 942 bp, Homo sapiens
gcacgaaagtcaaagaagttgggaaagttaaaagggattcacgaggagcagccacagcagcagcagcccccacccccacccccacccccgcaaagcccagaggaagggacaacgtacatcgctcctgcaaaagaaccctcggtcaacacagcactggttcctcagctctccacaatctcacgagcgctcacaccttcccccgttatggtccttgaaaacattgaacctgaaattgtatatgcaggctatgacagctcaaaaccagatacagccgaaaatctgctctccacgctcaaccgcttagcaggcaaacagatgatccaagtcgtgaagtgggcaaaggtacttccaggatttaaaaacttgcctcttgaggaccaaattaccctaatccagtattcttggatgtgtctatcatcatttgccttgagctggagatcgtacaaacatacgaacagccaatttctctattttgcaccagacctagtctttaatgaagagaagatgcatcagtctgccatgtatgaactatgccaggggatgcaccaaatcagccttcagttcgttcgactgcagctcacctttgaagaatacaccatcatgaaagttttgctgctactaagcacaattccaaaggatggcctcaaaagccaggctgcatttgaagaaatgaggacaaattacatcaaagaactgaggaagatggtaactaagtgtcccaacaattctgggcagagctggcagaggttctaccaactgaccaagctgctggactccatgcatgacctggtgagcgacctgctggaattctgcttctacaccttccgagagtcccatgcgctgaaggtagagttccccgcaatgctggtggagatcatcagcgaccagctgcccaaggtggagtcggggaacgccaagccgctctacttccaccggaagtga

SEQ ID NO: 5 - protein, 313 aa, Homo sapiens
ARKSKKLGKLKGIHEEQPQQQQPPPPPPPPQSPEEGTTYIAPAKEPSVNTALVPQLSTISRALTPSPVMVLENIEPEIVYAGYDSSKPDTAENLLSTLNRLAGKQMIQVVKWAKVLPGFKNLPLEDQITLIQYSWMCLSSFALSWRSYKHTNSQFLYFAPDLVFNEEKMHQSAMYELCQGMHQISLQFVRLQLTFEEYTIMKVLLLLSTIPKDGLKSQAAFEEMRTNYIKELRKMVTKCPNNSGQSWQRFYQLTKLLDSMHDLVSDLLEFCFYTFRESHALKVEFPAMLVEIISDQLPKVESGNAKPLYFHRK

SEQ ID NO: 6 - DNA, 441 bp, Homo sapiens
atgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcg

SEQ ID NO: 7 - DNA, 1404 bp, Homo sapiens
atgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgccggaattcccggggatccgtgcacgaaagtcaaagaagttgggaaagttaaaagggattcacgaggagcagccacagcagcagcagcccccacccccacccccacccccgcaaagcccagaggaagggacaacgtacatcgctcctgcaaaagaaccctcggtcaacacagcactggttcctcagctctccacaatctcacgagcgctcacaccttcccccgttatggtccttgaaaacattgaacctgaaattgtatatgcaggctatgacagctcaaaaccagatacagccgaaaatctgctctccacgctcaaccgcttagcaggcaaacagatgatccaagtcgtgaagtgggcaaaggtacttccaggatttaaaaacttgcctcttgaggaccaaattaccctaatccagtattcttggatgtgtctatcatcatttgccttgagctggagatcgtacaaacatacgaacagccaatttctctattttgcaccagacctagtctttaatgaagagaagatgcatcagtctgccatgtatgaactatgccaggggatgcaccaaatcagccttcagttcgttcgactgcagctcacctttgaagaatacaccatcatgaaagttttgctgctactaagcacaattccaaaggatggcctcaaaagccaggctgcatttgaagaaatgaggacaaattacatcaaagaactgaggaagatggtaactaagtgtcccaacaattctgggcagagctggcagaggttctaccaactgaccaagctgctggactccatgcatgacctggtgagcgacctgctggaattctgcttctacaccttccgagagtcccatgcgctgaaggtagagttccccgcaatgctggtggagatcatcagcgaccagctgcccaaggtggagtcggggaacgccaagccgctctacttccaccggaagtga

SEQ ID NO: 8 - DNA, 1965 bp, Homo sapiens
atgttgggggacggggattccccggggccgggatttaccccccacgactccgccccctacggcgctctggatatggccgacttcgagtttgagcagatgtttaccgatgcccttggaattgacgagtacggtggggaattcccggggatccgtcgacctgagaccaaaggctaccacagtctccctgaaggtctagatatggaaagacggtggggtcaagtttctcaggctgtggagcgttcttccctgggacctacagagaggaccgatgagaataactacatggagattgtcaacgtaagctgtgtttccggtgctattccaaacaacagtactcaaggaagcagcaaagaaaaacaagaactactcccttgccttcagcaagacaataatcggcctgggattttaacatctgatattaaaactgagctggaatctaaggaactttcagcaactgtagctgagtccatgggtttatatatggattctgtaagagatgctgactattcctatgagcagcagaaccaacaaggaagcatgagtccagctaagatttatcagaatgttgaacagctggtgaaattttacaaaggaaatggccatcgtccttccactctaagttgtgtgaacacgcccttgagatcatttatgtctgactctgggagctccgtgaatggtggcgtcatgcgcgccattgttaaaagccctatcatgtgtcatgagaaaagcccgtctgtttgcagccctctgaacatgacatcttcggtttgcagccctgctggaatcaactctgtgtcctccaccacagccagctttggcagttttccagtgcacagcccaatcacccagggaactcctctgacatgctcccctaatgctgaaaatcgaggctccaggtcgcacagccctgcacatgctagcaatgtgggctctcctctctcaagtccgttaagtagcatgaaatcctcaatttccagccctccaagtcactgcagtgtaaaatctccagtctccagtcccaataatgtcactctgagatcctctgtgtctagccctgcaaatattaacaactcaaggtgctctgtttccagcccttcgaacactaataacagatccacgctttccagtccggcagccagtactgtgggatctatctgtagccctgtaaacaatgccttcagctacactgcttctggcacctctgctggatccagtacattgcgggatgtggttcccagtccagacacgcaggagaaaggtgctcaagaggtcccttttcctaagactgaggaagtagagagtgccatctcaaatggtgtgactggccagcttaatattgtccagtacataaaaccagaaccagatggagcttttagcagctcatgtctaggaggaaatagcaaaataaattcggattcttcattctcagtaccaataaagcaagaatcaaccaagcattcatgttcaggcacctcttttaaagggaatccaacagtaaacccgtttccatttatggatggctcgtatttttcctttatggatgataaagactattattccctatcaggaattttaggaccacctgtgcccggctttgatggtaactgtgaaggcagcggattcccagtgggtattaaacaagaaccagatgacgggagctattacccagaggccagcatcccttcctctgctattgttggggtgaattcaggtggacagtccttccactacaggattggtgctcaaggtacaatatctttatcacgatcggctagagaccaatctttccaacacctgagttcctttcctcctgtcaatactttagtggagtcatggaaatcacacggcgacctgtcgtctagaagaagtgatgggtatccggtcttagaatacattccagaaaatgtatcaagctctactttacgaagtgtttctactggatcttcaaagcttctagataagtaa

SEQ ID NO: 9 - DNA, 17 bp, Artificial Sequence
  oligonucleotide
cggagtactgtcctccg

SEQ ID NO: 10 - DNA, 103 bp, Artificial Sequence
  oligonucleotide
cggagtactgtcctccgagcggagtactgtcctccgactcgagcggagtactgtcctccgatcggagtactgtcctccgcgaattccggagtactgtcctccg

SEQ ID NO: 11 - DNA, 714 bp, Homo sapiens
cctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatctccttaactattgcttccaaacatttttggataagaccatgagtattgaattccccgagatgttagctgaaatcatcaccaatcagataccaaaatattcaaatggaaatatcaaaaaacttctgtttcatcaaaagtga

SEQ ID NO: 12 - protein, 237 aa, Homo sapiens
PEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQK

SEQ ID NO: 13 - DNA, 1173 bp, Homo sapiens
atgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgccggaattcccggggatccctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatctccttaactattgcttccaaacatttttggataagaccatgagtattgaattccccgagatgttagctgaaatcatcaccaatcagataccaaaatattcaaatggaaatatcaaaaaacttctgtttcatcaaaagtga

(19) United States
(12) Patent Application Publication
(10) Pub. No.: US 2009/0054417 A1
(43) Pub. Date: Feb. 26, 2009

(54) COMPOUNDS AND COMPOSITIONS AS MODULATORS OF STEROID HORMONE NUCLEAR RECEPTORS

(75) Inventors: Pierre-Yves Michellys, San Marcos, CA (US); H. Michael Petrassi, Cardiff-by-the-Sea, CA (US); Wendy Richmond, San Diego, CA (US); Wei Pei, San Diego, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton (BM)

(21) Appl. No.: 11/572,903

(22) PCT Filed: Jul. 28, 2005
(86) PCT No.: PCT/US2005/027086

§ 371 Date: Jan. 22, 2008

Related U.S. Application Data
(60) Provisional application No. 60/592,076, filed on Jul. 28, 2004.

(51) Int. Cl. A61K 31/5415; C07D 265/36; A61K 31/538; A61K 31/498; A61P 5/00; C07D 241/36; C07D 279/16

(52) U.S. Cl. 514/224.2; 544/105; 514/230.5; 544/52; 544/354; 514/249

(57) ABSTRACT

The invention provides compounds, pharmaceutical compositions comprising such compounds and methods of using such compounds to treat or prevent diseases or disorders associated with the activation of steroid hormone nuclear receptors.

### CROSS-REFERENCE TO RELATED APPLICATIONS

This application claims the benefit of priority to U.S. Provisional Patent Application Nos. 60/592,076, filed 28 Jul. 2004. The full disclosure of this application is incorporated herein by reference in its entirety and for all purposes.

### BACKGROUND OF THE INVENTION

1. Field of the Invention

The invention provides compounds, pharmaceutical compositions comprising such compounds and methods of using such compounds to treat or prevent diseases or disorders associated with the activation of steroid hormone nuclear receptors.

2. Background

Steroid hormone receptors represent a subset of the nuclear hormone receptor superfamily. So named according to the cognate ligand which complexes with the receptor in its native state, the steroid hormone nuclear receptors include the glucocorticoid receptor (GR), the androgen receptor (AR), the mineralocorticoid receptor (MR), the estrogen receptor (ER), and the progesterone receptor (PR). MR is expressed in epithelial tissues, heart, kidneys, brain, vascular tissues and bone. Aldosterone is the endogenous ligand of MR and is primarily synthesized in the adrenal glands, heart, brain and blood vessels. Several detrimental effects are attributable to aldosterone, for example: sodium/water retention, renal fibrosis, vascular inflammation, vascular fibrosis, endothelial dysfunction, coronary inflammation, decrease in coronary blood flow, ventricular arrhythmias, myocardial fibrosis, ventricular hypertrophy and direct damage to cardiovascular systems, primarily the heart, vasculature and kidneys. Aldosterone action on all target organs is through activation of the MR receptor. GR is expressed in almost all tissues and organ systems and is crucial for the integrity of the function of the central nervous system and the maintenance of cardiovascular, metabolic, and immune homeostasis.

The novel compounds of the invention modulate the activity of the steroid hormone nuclear receptors and are, therefore, expected to be useful in the treatment of diseases in which aberrant activity of steroidal nuclear hormone receptors contributes to the pathology and/or symptomology of the disease.

### SUMMARY OF THE INVENTION

In one aspect, the present invention provides compounds of Formula I:

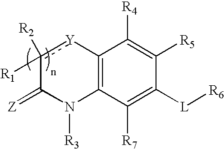

in which:

n is selected from 0, 1 and 2;

Z is selected from O and S;

Y is selected from O, S and NR₈; wherein R₈ is selected from hydrogen, C₁₋₆alkyl and halo-substituted-C₁₋₆alkyl;

L is selected from a bond, C₁₋₆alkylene, C₂₋₆alkenylene and C₂₋₆alkynylene; wherein any alkylene can be cyclized and alkylene or alkenylene of L can optionally have a methylene replaced with C(O), O, S(O)₀₋₂, and NR₉; wherein R₉ is selected from hydrogen and C₁₋₆alkyl, halo-substituted-C₁₋₆alkyl, C₆₋₁₀aryl, C₅₋₁₀heteroaryl, C₃₋₁₂cycloalkyl and C₃₋₈heterocycloalkyl; and wherein any alkylene or alkenylene of L is optionally substituted by 1 to 3 radicals independently selected from —C(O)OR₉ and C₁₋₆alkyl;

R₁ and R₂ are independently selected from hydrogen, halo and C₁₋₆alkyl;

R₃ is selected from hydrogen, C₁₋₆alkyl, —C(O)R₁₅ and —S(O)₀₋₂R₁₅; wherein R₁₅ is selected from hydrogen, C₁₋₆alkyl, cyano, nitro and halo-substituted-C₁₋₆alkyl, C₆₋₁₀aryl and C₅₋₁₀heteroaryl; wherein any ary or heteroaryl of R₉ is optionally substituted with 1 to 3 halo radicals;

R₄ is selected from hydrogen, halo, cyano, R₆, C₁₋₆alkyl, C₁₋₆alkylthio, halo-substituted-C₁₋₆alkyl, halo-substituted-C₁₋₆alkoxy and halo-substituted-C₁₋₆alkylthio;

R₅ and R₇ are independently selected from hydrogen, halo, C₁₋₆alkyl, C₁₋₆alkoxy, C₁₋₆alkylthio, halo-substituted-C₁₋₆alkyl, halo-substituted-C₁₋₆alkoxy and halo-substituted-C₁₋₆alkylthio;

R₆ is selected from C₆₋₁₅aryl, C₅₋₁₂heteroaryl, C₃₋₁₂cycloalkyl and C₃₋₈heterocycloalkyl; wherein any aryl, heteroaryl, cycloalkyl or heterocycloalkyl of R₆ is optionally substituted with 1 to 3 radicals independently selected from halo, hydroxy, amino, cyano, nitro, C₁₋₆alkyl, cyano-C₁₋₆alkyl, hydroxy-C₁₋₆alkyl, C₁₋₆alkoxy, C₁₋₆alkthio, halo-substituted-C₁₋₆alkyl, halo-substituted-C₁₋₆alkoxy, 2,2,2-trifluoro-1-hydroxy-ethyl, —XNR₁₀R₁₀, —XC(O)NR₁₀R₁₀, —XNR₁₀C(O)R₁₀, —XNR₁₀C(O)OXR₁₁, —XOR₁₀, —XOC(O)R₁₀, —XC(O)R₁₀, —XC(O)OR₁₀, —XS(O)₀₋₂NR₁₀R₁₀ and —NR₁₀R₁₁ and R₁₁; wherein each X is independently selected from a bond, C₁₋₆alkylene, C₂₋₆alkenylene and C₂₋₆alkynylene; each R₁₀ is independently selected from hydrogen and C₁₋₆alkyl; and R₁₁ is selected from C₆₋₁₀aryl, C₆₋₁₀aryl-C₁₋₄alkoxy, C₅₋₁₀heteroaryl, C₃₋₁₂cycloalkyl and C₃₋₈heterocycloalkyl; wherein any aryl, heteroaryl, cycloalkyl or heterocycloalkyl of R₁₁ is optionally substituted with 1 to 3 radicals independently selected from halo, cyano, hydroxy, —NR₁₀R₁₀, —NR₁₀C(O)R₁₀, —NR₁₀S(O)₀₋₂R₁₀, —NR₁₀-benzyl, C₁₋₆alkoxy, C₁₋₆alkyl and halo-substituted-C₁₋₆alkyl; in which R₁₀ is as described above;

with the proviso that if n is equal to zero, R₆ is not represented by Formula II:

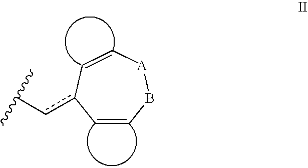

in which A and B are independently selected from O, S, C and NR₁₀; wherein R₁₀ is as described above; and the N-oxide derivatives, prodrug derivatives, protected derivatives, individual isomers and mixture of isomers thereof; and the pharmaceutically acceptable salts and solvates (e.g. hydrates) of such compounds.

In a second aspect, the present invention provides a pharmaceutical composition which contains a compound of Formula I or a N-oxide derivative, individual isomers and mixture of isomers thereof; or a pharmaceutically acceptable salt thereof, in admixture with one or more suitable excipients.

In a third aspect, the present invention provides a method of treating a disease in an animal in which modulation of steroid nuclear hormone receptor activities can prevent, inhibit or ameliorate the pathology and/or symptomology of the diseases, which method comprises administering to the animal a therapeutically effective amount of a compound of Formula I or a N-oxide derivative, individual isomers and mixture of isomers thereof, or a pharmaceutically acceptable salt thereof.

In a fourth aspect, the present invention provides the use of a compound of Formula I in the manufacture of a medicament for treating a disease in an animal in which steroid nuclear hormone receptor activity contributes to the pathology and/or symptomology of the disease.

In a fifth aspect, the present invention provides a process for preparing compounds of Formula I and the N-oxide derivatives, prodrug derivatives, protected derivatives, individual isomers and mixture of isomers thereof, and the pharmaceutically acceptable salts thereof.

### DETAILED DESCRIPTION OF THE INVENTION

### Definitions

“Alkyl” as a group and as a structural element of other groups, for example halo-substituted-alkyl and alkoxy, can be either straight-chained or branched. C₁₋₆alkoxy includes, methoxy, ethoxy, and the like. Halo-substituted alkyl includes trifluoromethyl, pentafluoroethyl, and the like.

“Aryl” means a monocyclic or fused bicyclic aromatic ring assembly containing six to ten ring carbon atoms. For example, aryl can be phenyl, naphthyl, 10,11-dihydro-5H-dibenzo[a,d]cycloheptene, and the like. “Arylene” means a divalent radical derived from an aryl group. “Heteroaryl” is as defined for aryl where one or more of the ring members are a heteroatom. For example heteroaryl includes pyridyl, indolyl, indazolyl, quinoxalinyl, quinolinyl, benzofuranyl, benzopyranyl, benzothiopyranyl, Benzo[1,2,5]oxadiazole, 3,4-Dihydro-2H-benzo[1,4]oxazine, 2,3-Dihydro-benzo[1,4]dioxine, Benzofuran, Benzo[1,3]dioxole, Benzo[b]thiophene, Benzo[1,3]dioxole, 1H-indazolyl, 9H-Thioxanthene, 6,11-Dihydro-dibenzo[b,e]oxepine, 8H-Indeno[1,2-d]thiazole, 5,6-Dihydro-4H-cyclopentathiazole, 4,5,6,7-Tetrahydro-benzothiazole, 4,5-Dihydro-2-oxa-6-thia-1,3,8-triaza-as-indacene, 1,2,3,4-Tetrahydro-isoquinoline, 4,5,6,7-Tetrahydro-thieno[2,3-c]pyridinebenzo[1,3]dioxole, imidazolyl, benzo-imidazolyl, pyrimidinyl, furanyl, oxazolyl, isoxazolyl, triazolyl, tetrazolyl, pyrazolyl, thienyl, etc. “C₆₋₁₀arylC₀₋₄alkyl” means an aryl as described above connected via a alkylene grouping. For example, C₆₋₁₀arylC₀₋₄alkyl includes phenethyl, benzyl, etc.

“Cycloalkyl” means a saturated or partially unsaturated, monocyclic, fused bicyclic or bridged polycyclic ring assembly containing the number of ring atoms indicated. For example, C₃₋₁₀cycloalkyl includes cyclopropyl, cyclobutyl, cyclopentyl, cyclohexyl, etc.

“Heterocycloalkyl” means cycloalkyl, as defined in this application, provided that one or more of the ring carbons indicated, are replaced by a moiety selected from —O—, —N═, —NR—, —C(O)—, —S—, —S(O)— or —S(O)₂—, wherein R is hydrogen, C₁₋₄alkyl or a nitrogen protecting group. For example, C₃₋₈heterocycloalkyl as used in this application to describe compounds of the invention includes morpholino, pyrrolidinyl, piperazinyl, piperidinyl, piperidinylone, 1,4-dioxa-8-aza-spiro[4.5]dec-8-yl, etc.

“Halogen” (or halo) preferably represents chloro or fluoro, but can also be bromo or iodo.

“Treat”, “treating” and “treatment” refer to a method of alleviating or abating a disease and/or its attendant symptoms.

### DESCRIPTION OF THE PREFERRED EMBODIMENTS

The present invention provides compounds, compositions and methods for the treatment of diseases, in which modulation of aberrant steroid nuclear hormone receptor activity can prevent, inhibit or ameliorate the pathology and/or symptomology of the diseases, which method comprises administering to the animal a therapeutically effective amount of a compound of Formula I.

In one embodiment of the invention, with respect to compounds of Formula I:

n is selected from 0 and 1;

Y is selected from O, S and NR₈; wherein R₈ is selected from hydrogen and C₁₋₆alkyl;

Z is selected from O and S;

L is selected from a bond, C₁₋₆alkylene, C₂₋₆alkenylene and C₂₋₆alkynylene; wherein any alkylene can be cyclized and alkylene or alkenylene of L can optionally have a methylene replaced with C(O), O, S(O)₀₋₂, and NR₉; wherein R₉ is selected from hydrogen and C₁₋₆alkyl, halo-substituted-C₁₋₆alkyl, C₆₋₁₀aryl, C₅₋₁₀heteroaryl, C₃₋₁₂cycloalkyl and C₃₋₈heterocycloalkyl; and wherein any alkylene or alkenylene of L is optionally substituted by 1 to 3 radicals independently selected from —C(O)OR₉ and C₁₋₆alkyl;

R₁ and R₂ are independently selected from hydrogen, halo and C₁₋₆alkyl;

R₃ is selected from hydrogen, C₁₋₆alkyl, —C(O)R₁₅ and —S(O)₀₋₂R₁₅; wherein R₁₅ is selected from hydrogen, C₁₋₆alkyl, cyano, nitro and halo-substituted-C₁₋₆alkyl, C₆₋₁₀aryl and C₅₋₁₀heteroaryl; wherein any ary or heteroaryl of R₉ is optionally substituted with 1 to 3 halo radicals;

R₄ is selected from hydrogen, halo, cyano, C₁₋₆alkyl and R₆;

R₅ and R₇ are independently selected from hydrogen, halo and C₁₋₆alkyl; and

R₆ is selected from C₆₋₁₅aryl, C₅₋₁₂heteroaryl, C₃₋₁₂cycloalkyl and C₃₋₈heterocycloalkyl; wherein any aryl, heteroaryl, cycloalkyl or heterocycloalkyl of R₆ is optionally substituted with 1 to 3 radicals independently selected from halo, hydroxy, amino, cyano, nitro, C₁₋₆alkyl, cyano-C₁₋₆alkyl, hydroxy-C₁₋₆alkyl, C₁₋₆alkoxy, C₁₋₆alkthio, halo-substituted-C₁₋₆alkyl, halo-substituted-C₁₋₆alkoxy, 2,2,2-trifluoro-1-hydroxy-ethyl, —XNR₁₀R₁₀, —XC(O)NR₁₀R₁₀, —XNR₁₀C(O)R₁₀, —XNR₁₀C(O)OXR₁₁, —XOR₁₀, —XOC(O)R₁₀, —XC(O)R₁₀, —XC(O)OR₁₀, —XS(O)₀₋₂NR₁₀R₁₀ and —NR₁₀R₁₁ and R₁₁; wherein each X is independently selected from a bond, C₁₋₆alkylene, C₂₋₆alkenylene and C₂₋₆alkynylene; each R₁₀ is independently selected from hydrogen and C₁₋₆alkyl; and R₁₁ is selected from C₆₋₁₀aryl, C₆₋₁₀aryl-C₁₋₄alkoxy, C₅₋₁₀heteroaryl, C₃₋₁₂cycloalkyl and C₃₋₈heterocycloalkyl; wherein any aryl, heteroaryl, cycloalkyl or heterocycloalkyl of R₁₁ is optionally substituted with 1 to 3 radicals independently selected from halo, cyano, hydroxy, —NR₁₀R₁₀, —NR₁₀C(O)R₁₀, —NR₁₀S(O)₀₋₂R₁₀, —NR₁₀-benzyl, C₁₋₆alkoxy, C₁₋₆alkyl and halo-substituted-C₁₋₆alkyl; in which R₁₀ is as described above.

In a further embodiment, R₄ is selected from hydrogen, halo, methyl and R₆; and R₇ is selected from hydrogen and methyl.

In a further embodiment, R₆ is selected from C₁₋₆alkyl, phenyl, thiazolyl, pyridinyl, indolyl, oxazolyl, Benzo[1,2,5]oxadiazole, 3,4-dihydro-2H-benzo[1,4]oxazine, 2,3-Dihydro-benzo[1,4]dioxine, 1H-indazolyl, 9H-thioxanthene, 6,11-dihydro-dibenzo[b,e]oxepine, 8H-indeno[1,2-d]thiazole, 5,6-dihydro-4H-cyclopentathiazole, 4,5,6,7-tetrahydro-benzothiazole, 4,5-dihydro-2-oxa-6-thia-1,3,8-triaza-as-indacene, 1,2,3,4-tetrahydro-isoquinoline, 4,5,6,7-tetrahydro-thieno[2,3-c]pyridine, naphthyl, thienyl, 1,2,3,4-tetrahydro-isoquinolinyl, 1,3-dihydro-isoindolyl, 3,4-dihydro-1H-isoquinolinyl, benzo[1,3]dioxolyl, benzo[b]furanyl, benzo[b]thienyl, benzo[1,2,5]oxadiazolyl, benzoxazolyl and 2,3-dihydro-benzo[1,4]dioxinyl; wherein R₁₀ is optionally substituted with 1 to 3 radicals independently selected from halo, methyl, trifluoromethyl, nitro, hydroxy, methyl-carbonyl-oxy, methoxy, cyano, ethyl, acetyl, methoxy-carbonyl, amino, amino-sulfonyl, methyl-carbonyl-methyl, dimethyl-amino, dimethylamino-sulfonyl, hydroxy-methyl and cyano-methyl.

Preferred compounds of Formula I are selected from the examples and tables, infra.

### Pharmacology and Utility

Compounds of the invention modulate the activity of steroidal nuclear hormone receptors and, as such, are useful for treating diseases or disorders in which aberrant steroidal nuclear hormone receptor activity contributes to the pathology and/or symptomology of the disease. The invention further provides compounds for use in the preparation of medicaments for the treatment of diseases or disorders in which steroidal nuclear hormone receptor activity contributes to the pathology and/or symptomology of the disease.

Mineralocorticoids and glucocorticoids exert profound influences on a multitude of physiological functions by virtue of their diverse roles in growth, development, and maintenance of homeostasis. Their actions are mediated by the MR and GR.

In visceral tissues, such as the kidney and the gut, MR regulates sodium retention, potassium excretion, and water balance in response to aldosterone. Elevations in aldosterone levels, or excess stimulation of mineralocorticoid receptors, are linked to several pathological disorders or pathological disease states including, Conn's Syndrome, primary and secondary hyperaldosteronism, increased sodium retention, increased magnesium and potassium excretion (diuresis), increased water retention, hypertension (isolated systolic and combined systolic/diastolic), arrhythmias, myocardial fibrosis, myocardial infarction, Barter's Syndrome, congestive heart failure (CHF), and disorders associated with excess catecholamine levels. In addition, MR expression in the brain appears to play a role in the control of neuronal excitability, in the negative feedback regulation of the hypothalamic-pituitary-adrenal axis, and in the cognitive aspects of behavioral performance. Further, aldosterone antagonists are useful in the treatment of subjects suffering from one or more cognitive dysfunctions including, but not limited to psychoses, cognitive disorders (such as memory disturbances), mood disorders (such as depression and bipolar disorder), anxiety disorders, and personality disorders. In particular, mineralocorticoid receptors, and modulation of MR activity, are involved in anxiety and major depression. Finally, expression of MR may be related to differentiation of breast carcinomas. Thus MR modulators may also have utility in treating cancer, particularly of the breast.

GR is expressed in almost all tissues and organ systems and is crucial for the integrity of the function of the central nervous system and the maintenance of cardiovascular, metabolic, and immune homeostasis. Glucocorticoids (e.g. cortisol, corticosterone, and cortisone), and the glucocorticoid receptor, have been implicated in the etiology of a variety of pathological disorders or pathologic disease states. For example, cortisol hypo-secretion is implicated in the pathogenesis of diseases resulting in muscle weakness, increased melanin pigmentation of the skin, weight loss, hypotension, and hypoglycemia. On the other hand, excessive or prolonged secretion of glucocorticoids has been correlated to Cushing's Syndrome and can also result in obesity, hypertension, glucose intolerance, hyperglycemia, diabetes mellitus, osteoporosis, polyuria, and polydipsia.

Further, GR selective agents could modulate GR activity and, thus, be useful in the treatment of inflammation, tissue rejection, auto-immunity, malignancies such as leukemias and lymphomas, Cushing's syndrome, acute adrenal insufficiency, congenital adrenal hyperplasia, rheumatic fever, polyarteritis nodosa, granulomatous polyarteritis, inhibition of myeloid cell lines, immune proliferation/apoptosis, HPA axis suppression and regulation, hypercortisolemia, modulation of the Th1/Th2 cytokine balance, chronic kidney disease, stroke and spinal cord injury, hypocalcaemia, hyperglycemia, acute adrenal insufficiency, chronic primary adrenal insufficiency, secondary adrenal insufficiency, congenital adrenal hyperplasia, cerebral edema, thrombocytopenia, and Little's syndrome. It has been reported that GR modulators are especially useful in disease states involving systemic inflammation such as inflammatory bowel disease, systemic lupus erythematosus, polyartitis nodosa, Wegener's granulomatosis, giant cell arthritis, rheumatoid arthritis, osteoarthritis, hay fever, allergic rhinitis, urticaria, angioneurotic edema, chronic obstructive pulmonary disease, asthma, tendonitis, bursitis, Crohn's disease, ulcerative colitis, autoimmune chronic active hepatitis, organ transplantation, hepatitis, and cirrhosis; and that GR modulating compounds have been used as immunostimulants, repressors, and as wound healing and tissue repair agents. In addition, GR modulators have also found use in a variety of topical diseases such as inflammatory scalp alopecia, panniculitis, psoriasis, discoid lupus erythematosus, inflamed cysts, atopic dermatitis, pyoderma gangrenosum, pemphigus vulgaris, bullous pemphigoid, systemic lupus erythematosus, dermatomyositis, eosinophilic fasciitis, relapsing polychondritis, inflammatory vasculitis, sarcoidosis, Sweet's disease, type 1 reactive leprosy, capillary hemangiomas, contact dermatitis, atopic dermatitis, lichen planus, exfoliative dermatitis, erythema nodosum, acne, hirsutism, toxic epidermal necrolysis, erythema multiform, and cutaneous T-cell lymphoma. Finally, GR Modulators may also have utility in treating respiratory disorders, such as emphysema, and neuroinflammatory disorders, such as multiple sclerosis and Alzheimer's disease.

Accordingly, the present invention provides a method for treating any of the diseases or disorders described above in a subject in need of such treatment, which method comprises administering to said subject a therapeutically effective amount (See, “Administration and Pharmaceutical Compositions”, infra) of a compound of Formula I or a pharmaceutically acceptable salt thereof. For any of the above uses, the required dosage will vary depending on the mode of administration, the particular condition to be treated and the effect desired.

### Administration and Pharmaceutical Compositions

In general, compounds of the invention will be administered in therapeutically effective amounts via any of the usual and acceptable modes known in the art, either singly or in combination with one or more therapeutic agents. A therapeutically effective amount can vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrollidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions can be sterilized and/or contain adjuvants, such as preserving, stabilizing, wetting or emulsifying agents, solution promoters, salts for regulating the osmotic pressure and/or buffers. In addition, they can also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations can also be used. Suitable formulations for topical application, e.g., to the skin and eyes, are preferably aqueous solutions, ointments, creams or gels well-known in the art. Such can contain solubilizers, stabilizers, tonicity enhancing agents, buffers and preservatives.

Compounds of the invention can be administered in therapeutically effective amounts in combination with one or more therapeutic agents (pharmaceutical combinations). For example, synergistic effects can occur with other substances used in the treatment of hypokalemia, hypertension, congestive heart failure, renal failure, in particular chronic renal failure, restenosis, atherosclerosis, syndrome X, obesity, nephropathy, post-myocardial infarction, coronary heart disease, increased formation of collagen, fibrosis and remodeling following hypertension and endothelial dysfunction. Examples of such compounds include anti-obesity agents, such as orlistat, anti-hypertensive agents, inotropic agents and hypolipidemic agents e.g., loop diuretics, such as ethacrynic acid, furosemide and torsemide; angiotensin converting enzyme (ACE) inhibitors, such as benazepril, captopril, enalapril, fosinopril, lisinopril, moexipril, perinodopril, quinapril, ramipril and trandolepril; inhibitors of the Na—K-ATPase membrane pump, such as digoxin; neutralendopeptidase (NEP) inhibitors; ACE/NEP inhibitors, such as omapatrilat, sampatrilat, and fasidotril; angiotensin II antagonists, such as candesartan, eprosartan, irbesartan, losartan, telmisartan and valsartan, in particularvalsartan; β-adrenergic receptor blockers, such as acebutolol, betaxolol, bisoprolol, metoprolol, nadolol, propanolol, sotalol and timolol; inotropic agents, such as digoxin, dobutamine and milrinone; calcium channel blockers, such as amlodipine, bepridil, diltiazem, felodipine, nicardipine, nimodipine, nifedipine, nisoldipine and verapamil; and 3-hydroxy-3-methyl-glutaryl coenzyme A reductase (HMG-CoA) inhibitors, such as lovastatin, pitavastatin, simvastatin, pravastatin, cerivastatin, mevastatin, velostatin, fluvastatin, dalvastatin, atorvastatin, rosuvastatin and rivastatin. Where the compounds of the invention are administered in conjunction with other therapies, dosages of the co-administered compounds will of course vary depending on the type of co-drug employed, on the specific drug employed, on the condition being treated and so forth.

The invention also provides for a pharmaceutical combinations, e.g. a kit, comprising a) a first agent which is a compound of the invention as disclosed herein, in free form or in pharmaceutically acceptable salt form, and b) at least one co-agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of Formula I and a co-agent, are both administered to a patient simultaneously in the form of a single entity or dosage. The term “non-fixed combination” means that the active ingredients, e.g. a compound of Formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

### Processes for Making Compounds of the Invention

The present invention also includes processes for the preparation of compounds of the invention. In the reactions described, it can be necessary to protect reactive functional groups, for example hydroxy, amino, imino, thio or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Chemistry”, John Wiley and Sons, 1991.

Compounds of Formula I, in which Y and Z are both oxygen, can be prepared by proceeding as in the following Reaction Scheme I:

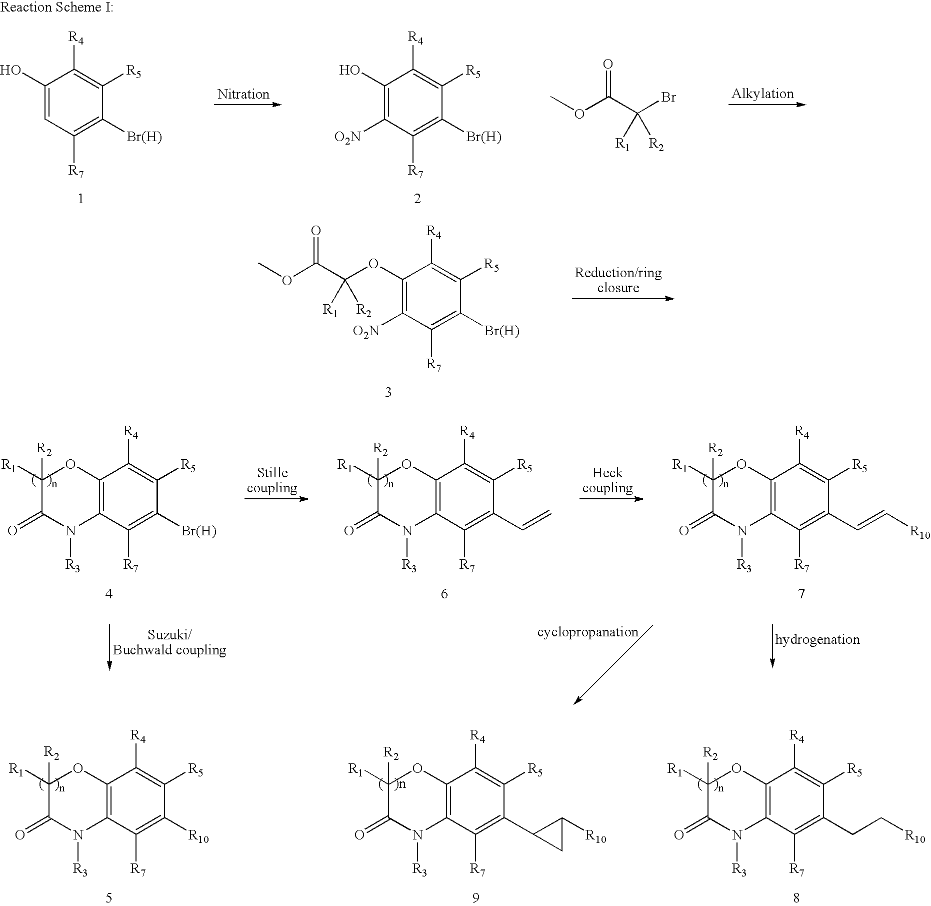

in which n, R₁, R₂, R₃, R₄, R₅, R₆, R₇ and R₁₀ are as defined for Formula I in the Summary of the Invention. Compounds of Formula I are prepared from phenolic derivatives (1). Nitration of (1), bearing either a proton or bromine substituent at the R₆ position, is accomplished with desired regiochemistry using ytterbium triflate (Synlett, 2000, 1, 57) as catalyst to afford the desired nitrophenols (2). The phenols are alkylated with methyl bromoacetate to afford ethers (3). Reduction of the nitro group with iron (Synthesis, 1993, 51) and acetic acid affords the desired benzoxazinone precursors (4) which can be subjected to a Suzuki or Buchwald coupling to afford the derivatives (5) or to a Stille coupling to give the vinyligous derivatives (6). Following a Heck coupling with various halogenated derivatives (6) affords the stilbene derivatives (7) that can be transformed into the corresponding cyclopropane derivatives (9) or phenethyl (8) by hydrogenation.

Compounds of Formula I, in which W is a heteroaryl group, can be synthesized according to reaction schemes II and III:

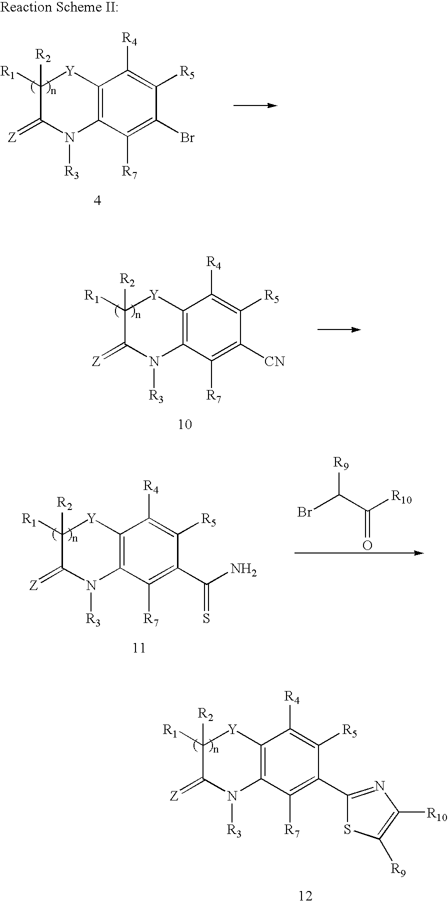

in which n, Y, Z, R₁, R₂, R₃, R₄, R₅, R₇, R₉ and R₁₀ are as defined for Formula I in the Summary of the Invention. Compounds of Formula I are prepared from 6-bromo-4H-benzo[1,4]oxazin-3-ones (4) by cyanation using Zn(CN)₂ and a palladium mediated coupling to afford 6-cyano-4H-benzo[1,4]oxazin-3-ones (10). The nitriles (10) are converted to the corresponding thioamides (11) via treatment with H₂S gas. The thioamides (11) are reacted with α-halo ketones to afford the desired thiazoles (12).

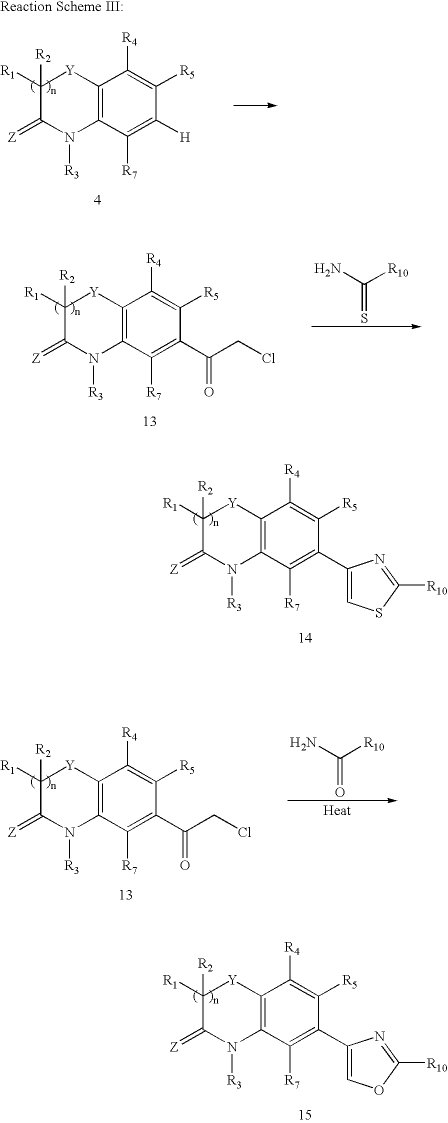

in which n, Y, Z, R₁, R₂, R₃, R₄, R₅, R₇ and R₁₀ are as defined for Formula I in the Summary of the Invention. Compounds of Formula I are prepared from 4H-benzo[1,4]oxazin-3-ones (4) by a Friedel crafts acylation with chloroacetyl chloride to afford the chloro ketones (13). The 6-(2-chloro-acetyl)-4H-benzo[1,4]oxazin-3-ones (13) are then reacted with a thioamide to afford the desired thiazole (14). Alternatively, thermolysis of derivatives (13) with an amide derivative affords the corresponding oxazole derivatives (15). Compounds of formula I where Y is S or NR₈ (wherein R₈ being as described above) may be synthesized from the following reaction scheme IV.

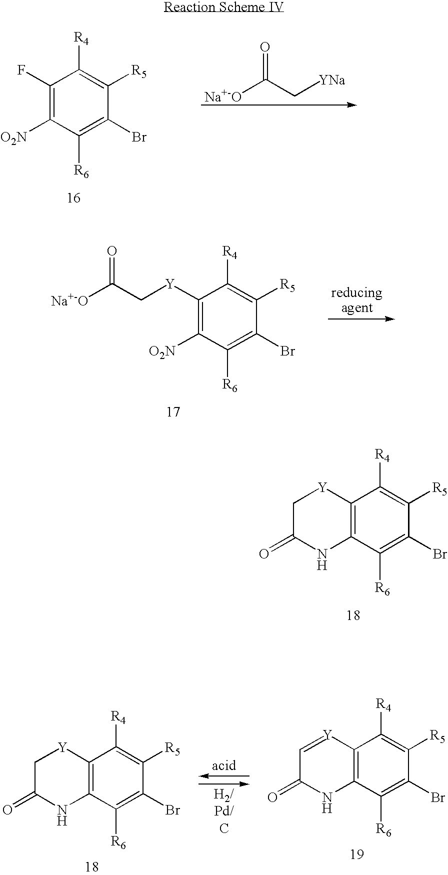

wherein a halo derivative 16 is subjected to an aromatic substitution with an anion to afford the deriavtive 17. The nitro group of 17 is then subjected to a reduction reaction (tin (II) chloride or the like) to give the derivative 13 that can easily be transformed into 19 in the presence of acid. Both 18 and 19 may be further utilized according to reaction scheme I, II and III.

Specific examples of synthesis of compounds of the invention are detailed, infra.

### Additional Processes for Making Compounds of the Invention

A compound of the invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the invention can be prepared by reacting the free acid form of the compound with a pharmaceutically acceptable inorganic or organic base. Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example a compound of the invention in an acid addition salt form can be converted to the corresponding free base by treating with a suitable base (e.g., ammonium hydroxide solution, sodium hydroxide, and the like). A compound of the invention in a base addition salt form can be converted to the corresponding free acid by treating with a suitable acid (e.g., hydrochloric acid, etc.).

Compounds of the invention in unoxidized form can be prepared from N-oxides of compounds of the invention by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3^{rd} edition, John Wiley and Sons, Inc., 1999.

Compounds of the present invention can be conveniently prepared, or formed during the process of the invention, as solvates (e.g., hydrates). Hydrates of compounds of the present invention can be conveniently prepared by recrystallization from an aqueous/organic solvent mixture, using organic solvents such as dioxin, tetrahydrofuran or methanol.

Compounds of the invention can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981.

In summary, the compounds of Formula I can be made by a process, which involves:

(a) that of reaction scheme I, II, III or IV; and

(b) optionally converting a compound of the invention into a pharmaceutically acceptable salt;

(c) optionally converting a salt form of a compound of the invention to a non-salt form;

(d) optionally converting an unoxidized form of a compound of the invention into a pharmaceutically acceptable N-oxide;

(e) optionally converting an N-oxide form of a compound of the invention to its unoxidized form;

(f) optionally resolving an individual isomer of a compound of the invention from a mixture of isomers;

(g) optionally converting a non-derivatized compound of the invention into a pharmaceutically acceptable prodrug derivative; and

(h) optionally converting a prodrug derivative of a compound of the invention to its non-derivatized form.

Insofar as the production of the starting materials is not particularly described, the compounds are known or can be prepared analogously to methods known in the art or as disclosed in the Examples hereinafter.

One of skill in the art will appreciate that the above transformations are only representative of methods for preparation of the compounds of the present invention, and that other well known methods can similarly be used.

### EXAMPLES

The present invention is further exemplified, but not limited, by the following reference examples (intermediates) and examples that illustrate the preparation of compounds of Formula I according to the invention.

### Reference 1

### Heck Coupling

A 40 mL scintillation vial is charged with 6-vinyl-4H-benzo[1,4]oxazin-3-one (30 mg, 0.17 mmol), Pd₂(dba)₃ (8 mg, 0.009° mmol) and [(t-Bu)₃PH]BF₄] (15 mg, 0.05 mmol) aryl halide (0.20 mmol), and Cy₂NMe (37 mL, 0.19 mmol) are added. The vial is then purged under a pressure of nitrogen and N-methylpyrrolidone (1 mL) is added via syringe and the reaction is stirred overnight (minimum 12 hours) at 110° C. under an atmosphere of nitrogen. After filtration through a nylon filter the product is purified from the reaction mixture by preparative LCMS.

### Reference 2

### Hydrogenation

To the ethyl acetate: methanol (2 to 3 mL, 3:1 v:v) solution of the alkene is added a catalytic amount of palladium on activated carbon (10 wt %, Aldrich # 20, 569-9) in a 40 mL scintillation vial. The vial is then evacuated and backfilled with hydrogen three times. Following the last hydrogen fill the reaction mixture is stirred overnight (minimum 12 hours) at room temperature under an atmosphere of hydrogen. After filtration through a nylon filter the product is purified from the reaction mixture by preparative LCMS. Alternatively, ammonium acetate may be used as hydrogen source instead of hydrogen gas.

### Reference 3

### Suzuki Coupling

A 40 mL scintillation vial is charged with the benzoxazinone halide (0.1 mmol), potassium phosphate (65 mg, 0.3 mmol), aryl boronic acid or pinicol ester (0.2 mmol), and chloro(di-2-norbornylphosphino)(2′-dimethylamino-1,1′-biphenyl-2-yl)palladium (II) (Strem 46-0270) (2.5 mg 0.05 mmol). The vial is then purged under a pressure of nitrogen and 1,4-dioxane (4 mL) is added via syringe and the reaction is stirred overnight (minimum 12 hours) at 95° C. under an atmosphere of nitrogen. The reaction is cooled to room temperature and then diluted with brine (10 mL) and ethyl acetate (4 mL). The layers are separated and the organic layer is concentrated under reduced pressure. The organic layers are dissolved in dimethylsulfoxide (DMSO) and, following filtration of the crude DMSO solution through a nylon filter, the product is purified from the reaction mixture by preparative LCMS.

In some cases, benzoxazinone pinicol ester is used (in lieu of a benzoxazinone halide) and a halobenzene (in lieu of a boronic acid or pinicol ester) is used the amounts of reagents are constant.

### Reference 4

### Alternate Heck Coupling

A 40 mL scintillation vial is charged with 6-bromo-4H-benzo[1,4]oxazin-3-one (38 mg, 0.17 mmol), Pd₂(dba)₃ (8 mg, 0.009 mmol) and [(t-Bu)₃PH]BF₄] (15 mg, 0.05 mmol) styrene (0.34 mmol), and Cy₂NMe (37 mL, 0.19 mmol) are added. The vial is then purged under a pressure of nitrogen and N-methylpyrrolidone (1 mL) is added via syringe and the reaction is stirred overnight (minimum 12 hours) at 110° C. under an atmosphere of nitrogen. After filtration through a nylon filter the product is purified from the reaction mixture by preparative LCMS.

### Reference 5

### Alternate Suzuki Coupling

A 40 mL scintillation vial is charged with the benzoxazinone halide (0.1 mmol), potassium phosphate (65 mg, 0.3 mmol), aryl boronic acid or pinicol ester (0.2 mmol), and chloro(di-2-norbornylphosphino)(2′-dimethylamino-1,1′-biphenyl-2-yl)palladium (II) (Strem 46-0270) (2.5 mg 0.05 mmol). The vial is then purged under a pressure of nitrogen and 1,4-dioxane (4 mL) is added via syringe and the reaction is stirred overnight (minimum 12 hours) at 95° C. under an atmosphere of nitrogen. The reaction is cooled to room temperature and then diluted with brine (10 mL) and ethyl acetate (4 mL). The layers are separated and the organic layer is concentrated under reduced pressure. The organic layers are dissolved in dimethylsulfoxide (DMSO) and following filtration of the crude DMSO solution through a nylon filter the product is purified from the reaction mixture by preparative LCMS.

In some cases, benzoxazinone pinicol ester is used (in lieu of a benzoxazinone halide) and a halobenzene (in lieu of a boronic acid or pinicol ester) is used the amounts of reagents are constant.

### Reference 6

### Hantzsch Thiazole Synthesis

To a vial are charged the α-haloketone (0.2 mmol), thioamide (0.2 mmol) and ethanol (2 mL). The reaction is heated to 180° C. for 10 min and then cooled to room temperature. The solvent is decanted off, the yellow residue is dissolved in DMSO and the product purified from the reaction mixture via preparative HPLC.

### Reference 7

### Acetate Cleavage

To a vial charged with the desired acetate was added methanol (2 ml per mmol) potassium carbonate (30 eq.). The reaction is stirred for 1 h at room temperature, quenched with water, filtered through celite and then the product is purified by preparative LCMS. Alternatively, a mixture of 3:1:1 THF/methanol/water and lithium hydroxide (4 eq.) may be used instead of K₂CO₃/MeOH. In this case, the reaction is stirred for 4 h at room temperature, neutralized with 1M HCl, and filtered through celite. The product is purified by preparative LCMS.

### Reference 8

### Buchwald Coupling

To a scintillation vial charged with the 6-bromo-4H-benzo[1,4]oxazin-3-one, Pd₂(dba)₃ (2.5% substrate), 2-(dicyclohexylphosphino)-2′-(N,N-dimethylamino)biphenyl (6% subatrate). The vial is purged under a positive flow of nitrogen and 1,4-dioxane, the amine and lithium hexamethyldisylazide (1 equivalent substrate) was added via syringe. The reaction is stirred for overnight at 90° C. under an atmosphere of nitrogen. Upon cooling the reaction is concentrated onto celite under reduced pressure and purified via flash column chromatography or by preparative LCMS.

The following examples of table 1 were synthesized according to reference 1.

TABLE 1

| | | Physical Data |
|---|---|---|
| | | ¹H NMR 400 MHz |
| Compound | | (CDCl₃ or DMSO) and/or |
| Number | Structure | MS (m/z) (M + 1)⁺ |
| 1 | 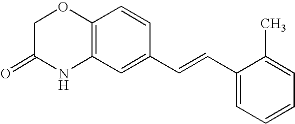 6-(2-o-tolyl-vinyl)-4H-benzo[1,4]oxazin-3-one | ¹H NMR (400 MHz,DMSO-_{d6}) δ 10.66 (s,1 H), 7.56-7.59 (m, 1 H),7.06-7.16, (m, 5 H), 7.03 (d,J = 2 Hz, 1 H), 6.97 (d, J =16 Hz, 1 H), 6.87 (d, J = 16Hz, 1 H) 4.51 (s, 2 H), 2.30(s, 3 H). MS: (ES⁺) 266 m/z(M + 1)⁺ C₁₇H₁₆NO₂ requires266 |
| 2 | 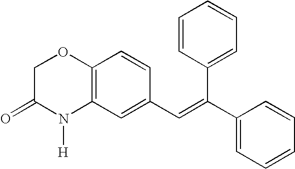 6-(2,2-Diphenyl-vinyl)-4H-benzo[1,4]oxazin-3-one | ¹H NMR (400 MHz,DMSO-_{d6}) δ 10.58 (s, 1 H),7.56-7.59 (m, 1 H), 7.17-7.34,(m, 8 H), 7.03-7.06(m, 2 H), 6.88 (s, 1 H),6.58-6.62 (m, 2 H), 6.38-6.41 (m,1 H), 4.43 (s, 2 H). MS:(ES⁺) 328 m/z (M + 1)⁺C₂₂H₁₈NO₂ requires 328 |
| 3 | 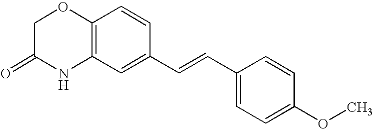 6-[2-(4-Methoxy-phenyl)-4H-benzo[1,4]oxazin-3-one | ¹H NMR (400 MHz,CDCl₃) δ 7.60 (s, 1 H), 7.43(d, J = 10.0 Hz, 2 H), 7.11(dd, J = 12.0 Hz, 9.9 Hz,1 H), 6.98-6.87 (m, 5 H),4.64 (s, 2 H), 3.84 (s, 3 H).MS: (ES⁺) 282 m/z (M + 1)⁺C₁₇H₁₅NO₃ requires 282 |
| 4 | 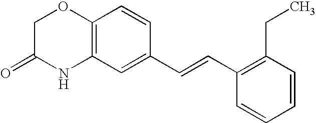 6-[2-(2-Ethyl-phenyl)-vinyl]-4H-benzo[1,4]oxazin-3-one | ¹H NMR (400 MHz,CDCl₃) δ 8.10 (s, 1 H), 8.03(d, J = 10.0 Hz, 2 H), 7.54(d, J = 10.0 Hz, 2 H), 7.18-7.10(m, 2 H), 7.03-6.95 (m,3 H), 4.65, (s, 2 H), 1.45 (q,J = 15.0 Hz, 2 H), 1.25 (t,J = 10.0 Hz, 3 H), 2.85-3.09(m, 4 H). MS: (ES⁺) 280m/z (M + 1)⁺ C₁₈H₁₇NO₂requires 280 |
| 5 | 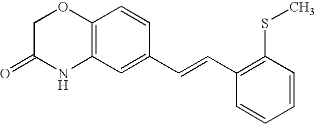 6-[2-(2-Methylsulvanyl-phenyl)-vinyl]-4H-benzo[1,4]oxazin-3-one | ¹H NMR (400 MHz,CDCl₃) δ 7.80 (s, 1 H), 7.55(d, J = 8.0 Hz, 1 H), 7.43 (d,J = 15.0 Hz, 1 H), 7.31-7.24(m, 2 H), 7.21-7.14 (m, 2 H),7.00-6.96 (m, 2 H), 6.95 (d,J = 16.0 Hz, 1 H), 4.65 (s,2 H), 2.57 (s, 3 H). MS:(ES⁺) 297 m/z (M + 1)⁺C₁₇H₁₄NO₂S requires 297 |
| 6 | 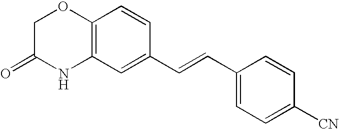 4-[2-(3-Oxo-3,4-dihydro-2H-benzo[1,4]oxazin-6-yl)-vinyl]-benzonitrile | ¹H NMR (400 MHz,CDCl₃) δ 7.87 (s, 1 H), 7.60(dd, J = 32.4 Hz, 8.4 Hz,4 H), 7.18-7.10 (m, 3 H),7.00-6.94 (m, 2 H), 4.6 (s,2 H). MS: (ES⁺) 307 m/z(M + 1)⁺ C₁₇H₁₂N₂O₂requires 307 |
| 7 | 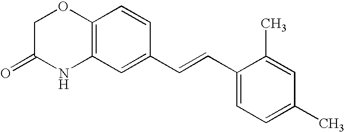 6-[2-(2-Dimethyl-phenyl)-vinyl]-4H-benzo[1,4]oxazin-3-one | ¹H NMR (400 MHz,CDCl₃) δ 7.60 (s, 1 H), 7.36(d, J = 7.6 Hz, 1 H), 7.10-7.02(m, 1 H), 6.91-6.86 (m,3 H), 6.82 (d, J = 2.0 Hz,1 H), 6.76 (d, J = 16.1 Hz,1 H), 4.54 (s, 2 H), 2.29 (s,3 H), 2.23 (s, 3 H). MS:(ES⁺) 280 m/z (M + 1)⁺C₁₈H₁₇NO₂ requires 280 |
| 8 | 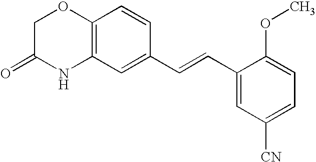 4-Methoxy-3-[2-(3-oxo-3,4-dihydro-2H-benzo[1,4]oxazin-6-yl)-vinyl]-benzonitrile | ¹H NMR (400 MHz,CDCl₃) δ 7.82 (d, J = 2.0Hz, 1 H), 7.66 (s, 1 H), 7.56-7.56(m, 1 H), 7.22 (s, 1 H),7.15 (dd, J = 2.0, 8.4 Hz,1 H), 6.94-7.02 (m, 4 H),4.65 (s, 2 H), 3.95 (s, 3 H).MS: (ES⁺) 307 m/z (M + 1)⁺C₁₈H₁₄N₂O₃ requires 307 |
| 9 | 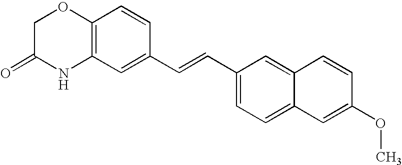 6-[2-(6-Methoxy-naphthalen-2-yl)-vinyl]-4H-benzo[1,4]oxazin-3-one | ¹H NMR (400 MHz,CDCl₃) δ 7.75 (s, 1 H),7.72-7.63 (m, 3 H), 7.47 (s,1 H), 7.16-7.10 (m, 3 H),7 07 (d, J = 6.0 Hz, 2 H),6.98-6.93 (m, 2 H), 4.63 (s,2 H), 3.91 (s, 3 H). MS:(ES⁺) 332 m/z (M + 1)⁺C₂₁H₁₇NO₃ requires 332 |
| 10 | 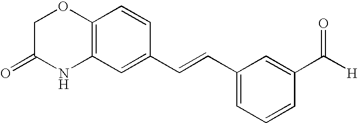 3-[2-(3-Oxo-3,4-dihydro-2H-benzo[1,4]oxazin-6-yl)-vinyl]-benzaldehyde | ¹H NMR (400 MHz,CDCl₃) δ 10.0 (s, 1 H), 7.96(m, 1 H), 7.87 (s, 1 H), 7.60(dd, J = 32.4 Hz, 8.4 Hz,4 H), 7.17 (d, J = 2.0 Hz,1 H), 7.15 (d, J = 2.0 Hz,1 H), 6.94-7.00 (m, 2 H).MS: (ES⁺) 280 m/z (M + 1)⁺C₁₇H₁₃NO₃ requires 280 |
| 11 | 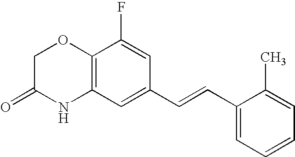 8-Fluoro-6-(2-o-tolyl-vinyl)-4H-benzo[1,4]oxazin-3-one | ¹H NMR (400 MHz,DMSO-_{d6}) δ 10.9 (s, 1 H),7.65 (m, 1 H), 7.30-7.26 (m,2 H), 7.20 (m, 3 H), 7.03 (d,J = 16.4 Hz, 1 H), 6.91 (s,1 H), 4.68 (s, 2 H), 2.39 (s,3 H). MS: (ES⁺) 284 m/z(M + 1)⁺ C₁₇H₁₄FNO₂ requires 284 |
| 12 | 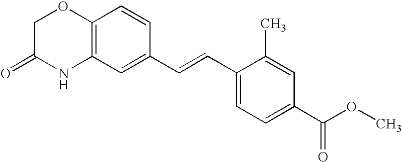 3-Methyl-4-[2-(3-oxo-3,4-dihydro-2H-benzoic[1,4]oxazin-6-yl)-vinyl]-benzo acid methyl ester | ¹H NMR (400 MHz,DMSO-_{d6}) δ 10.67 (s, 1 H),7.74-7.65 (m, 3 H), 7.17 (m,1 H), 7.13 (s, 2 H), 7.03 (d,J = 2.0 Hz, 1 H), 6.87 (d,J = 8.4 Hz, 1 H) 4.49 (s, 2 H),3.74 (s, 3 H), 2.34 (s, 3 H).MS: (ES⁺) 324 m/z (M + 1)⁺C₁₉H₁₇NO₄ requires 324 |
| 13 | 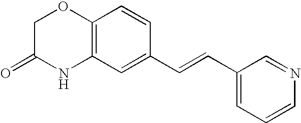 6-(2-Pyridin-3-yl-vinyl)-4H-benzo[1,4]oxazin-3-one | ¹H NMR (400 MHz,DMSO-_{d6}) δ 10.67 (s, 1 H),7.74-7.65 (m, 3 H), 7.17 (m,1 H), 7.13 (s, 2 H), 7.03 (d,J =2.0 Hz, 1 H), 6.87 (d, J =8.4 Hz, 1 H) 4.49 (s, 2 H),3.74 (s, 3 H), 2.34 (s, 3 H).MS: (ES⁺) 253 m/z (M + 1)⁺C₁₅H₁₂N₂O₂ requires 253 |
| 14 | 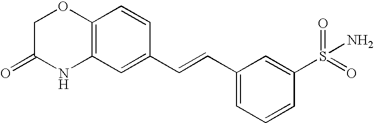 3-[2-(3-Oxo-3,4-dihydro-2H-benzo[1,4]oxazin-6-yl)-vinyl]-benzenesulfonamide | ¹H NMR (400 MHz,DMSO-_{d6}) δ 10.59 (s, 1 H),7.79 (dd, J = 4.0, 7.6 Hz,2 H), 7.40 (t, J = 8.4 Hz,1 H), 7.32 (t, J = 8.4 Hz,1 H), 7.09 (d, J = 2.4 Hz,1 H), 7.00-6.95 (m, 2 H),7.79 (d, J = 8.4 Hz, 1 H),4.42 (s, 2 H); MS: (ES⁺) 331m/z (M + 1)⁺C₁₆H₁₅N₂O₄S requires 331 |
| 15 | 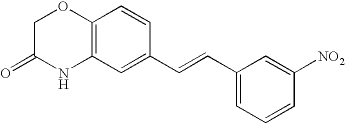 6-[2-(3-Nitro-phenyl)-vinyl]-4H-benzo[1,4]oxazin-3-one | ¹H NMR (400 MHz,DMSO-_{d6}) δ 10.86 (s, 1 H),8.43 (t, J = 1.6 Hz, 1 H),8.07 (t, J = 8.0 Hz, 2 H),7.65 (t, J = 8.0 Hz, 1 H),7.44 (d, J = 16.4 Hz, 1 H),7.26 (dd, J = 2.0, 8.4 Hz,1 H), 7.20 (d, J = 16.4 Hz,1 H), 7.10 (d, J = 6.0 Hz,1 H), 7.00 (d, J = 8.4 Hz,1 H), 4.60 (s, 2 H); MS:(ES⁺) 297 m/z (M + 1)⁺C₁₆H₁₃N₂O₄ requires 297 |
| 16 | 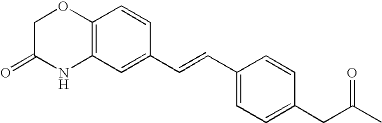 6-{2-(2-Oxo-propyl[-phenyl)-vinyl}-4H-benzo[1,4]oxazin-3-one | . ¹H NMR (400 MHz,DMSO-_{d6}) δ 10.78 (s, 1 H),7.53 (d, J = 8 Hz, 2 H),7.20-6.95 (m, 7 H), 4.59 (s,2 H), 3.80 (s, 2 H), 2.14 (s,3 H); MS: (ES⁺) 308 m/z(M + 1)⁺C₁₉H₁₈NO₃ requires 308 |
| 17 | 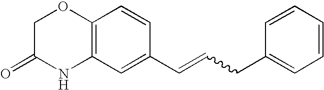 6-(3-Phenyl-propenyl)-4H-benzo[1,4]oxazin-3-one | MS: (ES⁺) 266 m/z (M + 1)⁺C₁₇H₁₆NO₂ requires 266 |
| 18 | 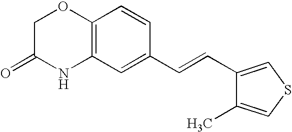 6-[2-(4-Methyl-thiophen-3-yl)-vinyl]-4H-benzo[1,4]oxazin-3-one | ¹H NMR (400 MHz,DMSO-_{d6}) δ 10.74 (s, 1 H),7.70 (d, J = 2.8 Hz, 1 H),7.19-7.16 (m, 1 H), 7.05 (d,J = 2.0 Hz, 1 H), 7.06-6.91(m, 4 H), 4.58 (s, 2 H), 2.28(s, 1 H); MS: (ES⁺) 272 m/z(M + 1)⁺C₁₅H₁₄NO₂S requires 272 |
| 19 | 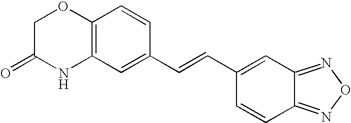 6-(2-Benzo[1,2,5]oxadiazol-5-yl-vinyl)-4H-benzo[1,4]oxazin-3-one | ¹H NMR (400 MHz,DMSO-_{d6}) δ 10.89 (s, 1 H),8.11-8.04 (m, 3 H), 7.56 (d,J =16.4 Hz, 1 H), 7.31-7.23(m, 2 H), 7.15 (d, J = 2.0Hz, 1 H), 7.02 (d, J = 8.4Hz, 1 H), 4.63 (s, 2 H); MS:(ES⁺) 294 m/z (M + 1)⁺C₁₆H₁₂N₃O₃ requires 294 |
| 20 | 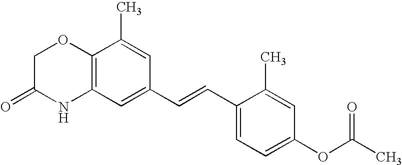 Acetic acid 3-methyl-4-[2-(8-methyl-3-oxo-3,4-dihydro-2H-benzo[1,4]oxazin-6-yl)-vinyl]-phenyl ester | ¹H NMR (400 MHz,DMSO-_{d6}) δ 10.67 (s, 1 H),7.67 (d, J = 8.4 Hz, 1 H),7.15 (d, J = 16.2 Hz, 1 H),7.12 (s, 1 H), 7.00-6.93 (m,4 H), 4.57 (s, 2 H), 2.38 (s,3 H), 2.26 (s, 3 H), 2.19 (s,3 H); MS: (ES⁺) 338 m/z(M + 1)⁺C₂₀H₂₀NO₄ requires 338 |
| 21 | 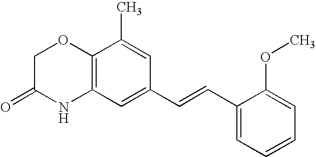 6-[2-(2-Methoxy-phenyl)-vinyl]-8-methyl-4H-benzo[1,4]oxazin-3-one | . ¹H NMR (400 MHz,DMSO-_{d6}) δ 10.63 (s, 1 H),7.63-7.61 (m, 1 H), 7.25-7.19(m, 1 H), 7.08-6.92 (m,6 H), 4.58 (s, 2 H), 3.59 (s,3 H), 2.18 (s, 3 H); MS:(ES⁺) 296 m/z (M + 1)⁺C₁₈H₁₈NO₃ requires 296 |
| 22 | 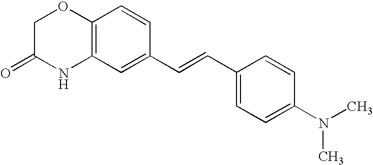 6-[2-(4-Dimethylamino-phenyl)-vinyl]-4H-benzo[1,4]oxazin-3-one | ¹H NMR (400 MHz,DMSO-_{d6}) δ 10.63 (s, 1 H),7.37 (d, J = 8.8 Hz, 2 H),7.05-7.03 (m, 1 H), 6.95 (d,J = 1.2 Hz, 1 H), 6.88-6.85(m, 3 H), 6.72-6.71 (m, 2 H),6.61 (d, J = 16.8 Hz, 1 H),4.56 (s, 2 H), 2.88 (s, 6 H);MS: (ES⁺) 295 m/z (M + 1)⁺C₁₈H₁₉N₂O₂ requires 295 |
| 23 | 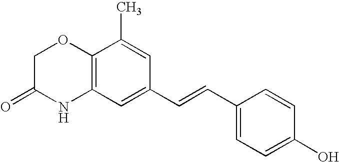 6-[2-(4-Hydroxy-phenyl)-vinyl]-8-methyl-4H-benzo[1,4]oxazin-3-one | ¹H NMR (400 MHz,DMSO-_{d6}) δ 10.68 (s, 1 H),9.55 (s, 1 H), 7.38 (d, J =8.8 Hz, 1 H), 7.02 (s, 1 H),6.89 (s, 1 H), 6.85 (d, J =2.0 Hz, 1 H), 6.74 (d, J =8.8 Hz, 1 H), 4.58 (s, 2 H),2.17 (s, 3 H); MS: (ES⁺) 282m/z (M + 1)⁺C₁₇H₁₆NO₃ requires 282 |
| 24 | 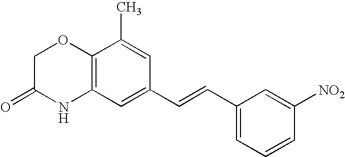 8-Methyl-6-[2-(3-nitro-phenyl)-vinyl]-4H-benzo[1,4]oxazin-3-one | ¹H NMR (400 MHz,DMSO-_{d6}) δ 11.02 (s, 1 H),8.64 (s, 1 H), 8.29 (t, J = 8.8Hz, 2 H), 7.87 (t, J = 8.0Hz, 1 H), 7.62 (d, J = 16.4Hz, 2 H), 7.44-7.40 (m, 2 H),7.17 (s, 1 H), 4.84 (s, 2 H),2.40 (s, 3 H); MS: (ES⁺)311 m/z (M + 1)⁺C₁₇H₁₅N₂O₄ requires 311 |
| 25 | 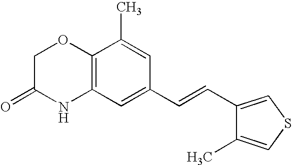 8-Methyl-6-[2-(4-methyl-thiophen-3-yl)-vinyl]-4H-benzo[1,4]oxazin-3-one | ¹H NMR (400 MHz,DMSO-_{d6}) δ 10.59 (s, 1 H),7.61 (d, J = 3.2 Hz, 1 H),7.09 (d, J = 2.4 Hz, 1 H),7.00 (s, 1 H), 6.85 (d, J =2.0 Hz, 2 H), 6.80 (d, J =2.0 Hz, 1 H), 4.51 (s, 2 H),2.19 (s, 3 H), 2.10 (s, 3 H);MS: (ES⁺) 286 m/z (M + 1)⁺C₁₆H₁₆NO₂S requires 286 |
| 26 | 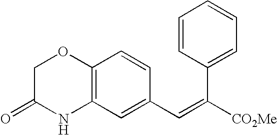 3-(3-Oxo-3,4-dihydro-2H-benzo[1,4]oxazin-6-yl)-2-phenyl-acrylicacid methyl ester | ¹H NMR (400 MHz,DMSO-_{d6}) δ 10.74 (s, 1 H),7.66 (s, 1 H), 7.41-7.36 (m,3 H), 7.16 (dd, J = 2.0, 8.0Hz, 2 H), 6.75 (d, J = 8.0Hz, 1 H), 6.67 (s, 1 H), 6.54(dd, J = 1.6, 7.6 Hz, 1 H),4.55 (s, 2 H), 3.69 (s, 3 H);MS: (ES⁺) 310 m/z (M + 1)⁺C₁₈H₁₆NO₄ requires 310 |
| 27 | 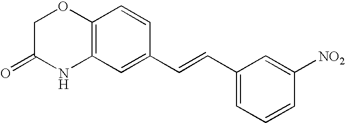 6-[2-(3-Nitro-phenyl)-vinyl]-4H-benzo[1,4]oxazin-3-one | . ¹H NMR (400 MHz,DMSO-_{d6}) δ 10.86 (s, 1 H),8.42 (s, 1 H), 8.09 (t, J = 7.6Hz, 2 H), 7.65 (t, J = 8.0Hz, 2 H), 7.43 (d, J = 16.8Hz, 1 H), 7.26 (dd, J = 1.6,8.0 Hz, 1 H), 7.19 (d, J =16.8 Hz, 1 H), 7.11 (d, J =2.0 Hz, 1 H), (d, J = 8.0 Hz,1 H), 4.60 (s, 2 H); MS:(ES⁺) 297 m/z (M + 1)⁺C₁₆H₁₃N₂O₄ requires 297 |
| 28 | 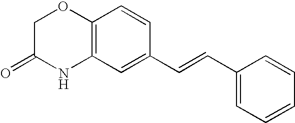 6-Styryl-4H-benzo[1,4]oxazin-3-one | . ¹H NMR (400 MHz,DMSO-_{d6}) δ 10.79 (s, 1 H),7.58 (d, J = 7.2 Hz, 1 H),7.36 (t, J = 5.2 Hz, 1 H),7.25 (t, J = 7.2 Hz, 1 H),7.21-7.16 (m, 2 H), 7.09-6.95(m, 3 H), 4.59 (s, 2 H);MS: (ES⁺) 252 m/z (M + 1)⁺C₁₆H₁₄NO₂ requires 252 |
| 29 | 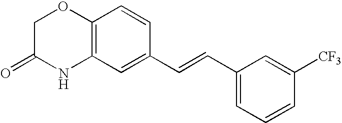 6-[2-(3-Trifluoromethyl-phenyl)-vinyl]-4H-benzo[1,4]oxazin-3-one | ¹H NMR (400 MHz,DMSO-_{d6}) δ 10.85 (s, 1 H),7.96 (s, 1 H), 7.91 (t, J = 3.6Hz, 1 H), 7.59 (d, J = 5.2Hz, 1 H), 7.38 (d, J = 16.8Hz, 1 H), 7.4 (dd, J = 1.6,8.4 Hz, 1 H), 7.16-7.09 (m,2 H), 6.98 (d, J = 8.4 Hz,1 H), 4.60 (s, 2 H); MS:(ES⁺) 320 m/z (M + 1)⁺C₁₇H₁₃F₂NO₂ requires 320 |
| 30 | 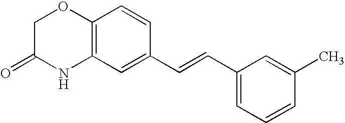 6-(2-m-Tolyl-vinyl)-4H-benzo[1,4]oxazin-3-one | ¹H NMR (400 MHz,DMSO-_{d6}) δ 10.79 (s, 1 H),7.41 (s, 1 H), 7.36 (d, J =8.0 Hz, 1 H), 7.25 (t, J = 7.6Hz, 1 H), 7.19-7.14 (m, 2 H),7.08-7.06 (m, 2 H), 7.00-6.94(s, 2 H), 4.59 (s, 2 H),2.32 (s, 3 H); MS: (ES⁺) 266m/z (M + 1)⁺C₁₇H₁₆NO₂ requires 266 |
| 31 | 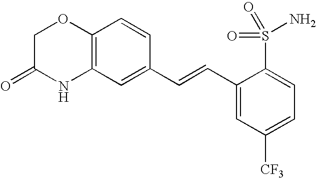 [2-(3-Oxo-3,4-dihydro-2H-benzo[1,4]oxazin-6-yl)-vinyl]-4-trifluoromethyl-benzenesulfonamide | . ¹H NMR (400 MHz,DMSO-_{d6}) δ 11.13 (s, 1 H),8.53 (s, 1 H), 8.33 (d, J =8.8 Hz, 1 H), 8.07-8.02 (m,3 H), 8.00 (d, J = 16 Hz,1 H), 7.71 (d, J = 16 Hz,1 H), 7.51 (dd, J = 2.0, 8.4Hz, 1 H), 7.42 (d, J = 2.0Hz, 1 H), 7.26 (d, J = 8.4Hz, 1 H), 4.86 (s, 2 H); MS:(ES⁺) 399 m/z (M + 1)⁺C₁₇H₁₄F₃N₂O₄S requires 399 |
| 32 | 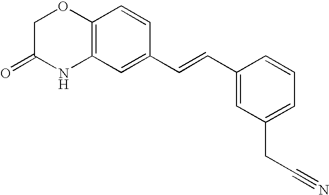 {2-[2-(3-Oxo-3,4-dihydro-2H-benzo[1,4]oxazin-6-yl)-vinyl]-phenyl}-acetonitrile | . ¹H NMR (400 MHz,CDCl₃) δ 7.87 (s, 1 H), 7.46(s, 1 H), 7.45 (d, J = 10.0Hz, 1 H), 7.37 (dd, J = 8.0Hz, 8.0 Hz, 1 H), 7.25 (d,J = 8.0 Hz, 1 H), 7.15 (dd,J = 9.2 Hz, 2.1 Hz, 1 H),7.03-6.94 (m, 4 H), 4.65 (s,2 H), 3.78 (s, 2 H). MS:(ES⁺) 291 m/z (M + 1)⁺C₁₈H₁₄N₂O₂ requires 291 |
| 33 | 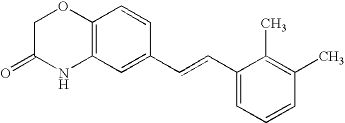 6-[2-(2,3-Dimethyl-phenyl)-vinyl]-4H-benzo[1,4]oxazin-3-one | . ¹H NMR (400 MHz,CDCl₃) δ 7.70 (s, 1 H),7.40-7.37 (m, 1 H), 7.30 (s,1 H), 7.14 (dd, J = 8.1 Hz,2.0 Hz, 1 H), 7.12 (d, J =4.0 Hz, 1 H), 7.10 (s, 1 H),6.97 (d, J = 10.2 Hz, 1 H),6.93 (d, J = 2.5 Hz, 1 H),6.82 (d, J = 16.1 Hz, 1 H)4.65 (s, 2 H), 2.34 (s, 6 H).MS: (ES⁺) 280 m/z (M + 1)⁺C₁₈H₁₇NO₂ requires 280 |
| 34 | 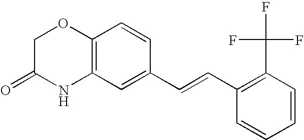 6-[2-(2-Trifluoromethyl-phenyl)-vinyl]-4H-benzo[1,4]oxazin-3-one | ¹H NMR (400 MHz,CDCl₃) δ 7.75 (d, J = 8.0Hz, 1 H), 7.67 (d, J = 7.2Hz, 1 H), 7.57-7.52 (m, 2 H),7.40-7.30 (m, 2 H), 7.15(dd, J = 8.4 Hz, 2.0 Hz, 1 H)7.01-6.94 (m, 3 H), 4.65 (s,2 H). MS: (ES⁺) 320 m/z(M + 1)⁺C₁₇H₁₂F₃NO₂ requires 320 |
| 35 | 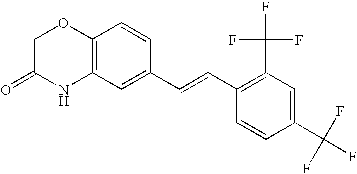 6-[2-(2,4-Bis-trifluoromethyl-phenyl)-vinyl]-4H-benzo[1,4]oxazin-3-one | ¹H NMR (400 MHz,CDCl₃) δ 7.94 (s, 1 H), 7.89(d, J = 8.4 Hz, 1 H), 7.80(dd, J = 9.2 Hz, 2.0 Hz,1 H), 7.53 (s, 1 H), 7.35 (s,1 H), 7.20-7.14 (m, 1 H),7.10-6.96 (m, 3 H), 4.65 (s,2 H). MS: (ES⁺) 388 m/z(M + 1)⁺C₁₈H₁₁F₆NO₂ requires 388 |
| 36 | 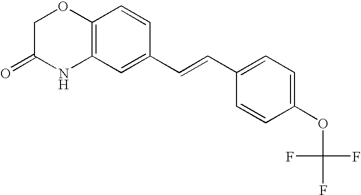 6-[2-(4-Trifluoromethoxy-phenyl)-vinyl]-4H-benzo[1,4]oxazin-3-one | ¹H NMR (400 MHz,CDCl₃) δ 7.81 (s, 1 H),7.51-7.49 (m, 2 H), 7.20 (d,J = 7.6 Hz, 2 H), 7.13 (dd,J = 8.4 Hz, 2.0 Hz, 1 H),7.00-6.91 (m, 4 H), 4.65 (s,2 H). MS: (ES⁺) 336 m/z(M + 1)⁺C₁₇H₁₂F₃NO₃ requires 336 |
| 37 | 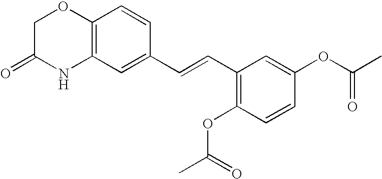 Acetic acid 4-acetoxy-3-[2-(3-oxo-3,4-dihydro-2H-benzo[1,4]oxazin-6-yl)-vinyl]-phenyl ester | . ¹H NMR (400 MHz,CDCl₃) δ 7.49 (s, 1 H), 7.37(d, J = 2.0 Hz, 1 H), 7.14-7.08(m, 2 H), 7.02-6.93 (m,4 H), 6.87 (d, J = 1.6 Hz,1 H), 4.65 (s, 2 H), 2.38 (s,3 H), 2.32 (s, 3 H). MS:(ES⁺) 368 m/z (M + 1)⁺C₂₀H₁₇NO₆ requires 368 |
| 38 | 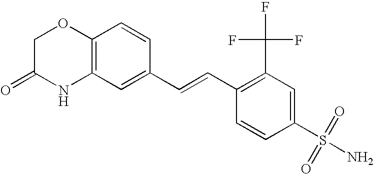 4-[2-(3-Oxo-3,4-dihydro-2H-benzo[1,4]oxazin-6-yl)-vinyl]-3-trifluoromethyl-benzenesulfonamide | ¹H NMR (400 MHz,DMSO-_{d6}) δ 10.80 (s, 1 H),8.25 (d, J = 8.4 Hz, 1 H),8.14-8.11 (m, 1 H), 8.06 (d,J = 8.8 Hz, 1 H), 7.59-7.57(m, 1 H), 7.52-7.47 (m, 1 H),7.22-7.18 (m, 2 H), 7.20 (d,J = 9.2 Hz, 1 H), 4.62 (s,2 H). MS: (ES⁺) 399 m/z(M + 1)⁺C₁₇H₁₃F₃N₂O₄S requires 399 |
| 39 | 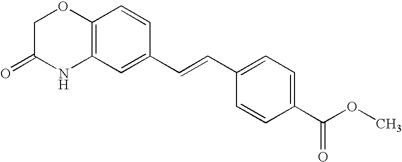 4-[2-(3-Oxo-3,4-dihydro-2H-benzo[1,4]oxazin-6-yl)-vinyl]-benzoicacid methyl ester | . ¹H NMR (400 MHz,DMSO-_{d6}) δ 10.82 (s, 1 H),7.96-7.92 (m, 2 H), 7.74 (d,J = 8.4 Hz, 2 H), 7.37 (d,J = 16.4 Hz, 1 H), 7.25 (dd,J = 8.4 Hz, 2.0 Hz, 1 H), 7.11(dd, J = 9.2 Hz, 7.2 Hz,2 H), 6.98 (d, J = 8.0 Hz,1 H), 4.62 (s, 2 H), 3.86 (s,3 H). MS: (ES⁺) 310 m/z(M + 1)⁺C₁₈H₁₅NO₄ requires 310 |
| 40 | 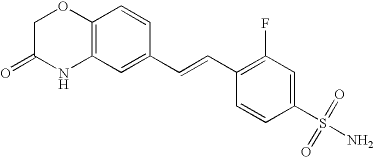 3-Fluoro-4-[2-(3-oxo-3,4-dihydro-2H-benzo[1,4]oxazin-6-yl)-vinyl]-benzenesulfonamide | ¹H NMR (400 MHz,DMSO-_{d6}) δ 10.80 (s, 1 H),8.03 (dd, J = 8.0 Hz, 8.0Hz, 1 H), 7.66-7.59 (m, 2 H),7.50 (s, 2 H), 7.43 (d, J =16.4 Hz, 1 H), 7.25 (dd, J =8.0 Hz, 1.6 Hz, 1 H), 7.16-7.14(m, 1 H) 7.11 (d, J =16.4 Hz, 1 H), 6.99 (d, J =8.4 Hz, 1 H), 4.62 (s, 2 H).MS: (ES⁺) 310 m/z (M + 1)⁺C₁₈H₁₅NO₄ requires 310 |
| 41 | 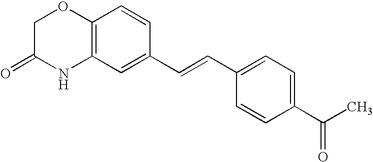 6-[2-(4-Acetyl-phenyl)-vinyl]-4H-benzo[1,4]oxazin-3-one | ¹H NMR (400 MHz,DMSO-_{d6}) δ 10.81 (s, 1 H),7.94 (d, J = 8.4 Hz, 2 H),7.73 (d, J = 8.4 Hz, 2 H),7.38 (d, J = 16.4 Hz, 1 H),7.25 (dd, J = 8.4 Hz, 2.4Hz, 1 H), 7.13 (dd, J = 10.0Hz, 8.4 Hz, 1 H), 4.62 (s,2 H), 2.57 (s, 3 H). MS:(ES⁺) 294 m/z (M + 1)⁺C₁₈H₁₅NO₃ requires 294 |
| 42 | 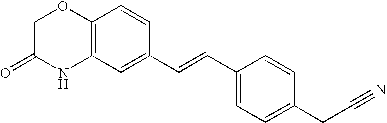 {4-[2-(3-Oxo-3,4-dihydro-2H-benzo[1,4]oxazin-6-yl)-vinyl]-phenyl}-acetonitrile | . ¹H NMR (400 MHz,DMSO-_{d6}) δ 10.80 (s, 1 H),7.61 (d, J = 8.4 Hz, 2 H),7.32 (d, J = 8.4 Hz, 2 H),7.21 (dd, J = 8.0 Hz, 6.0Hz, 1 H), 7.18 (s, 1 H), 7.08(d, J = 1.6 Hz, 1 H), 7.03 (d,J = 16.4 Hz, 1 H), 6.96 (d,J = 8.4 Hz, 1 H), 4.60 (s, 2 H),4.04 (s, 2 H). MS: (ES⁺)291 m/z (M + 1)⁺C₁₈H₁₄N₂O₂ requires 291 |
| 43 | 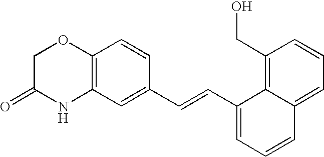 6-[2-(8-Hydroxymethyl-naphthalen-1-yl)-vinyl]-4H-benzo[1,4]oxazin-3-one | ¹H NMR (400 MHz,DMSO-_{d6}) δ 10.80 (s, 1 H),8.19 (d, J = 16.0 Hz, 1 H),7.89 (d, J = 7.6 Hz, 2 H),7.66-7.60 (m, 2 H), 7.52-7.44(m, 2 H), 7.23 (dd, J =8.0 Hz, 1.6 Hz, 1 H), 7.13(d, J = 2.0 Hz, 1 H), 7.00 (d,J = 8.4 Hz, 1 H), 6.82 (d,J = 16.0 Hz, 1 H), 5.52-5.48(m, 1 H), 4.93 (d, J = 5.2Hz, 2 H), 4.60 (s, 2 H). MS:(ES⁺) 332 m/z (M + 1)⁺C₂₁H₁₇NO₃ requires 332 |
| 44 | 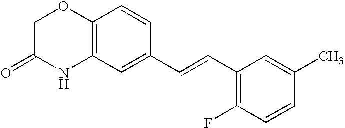 6-[2-(2-Fluoro-5-methyl-phenyl)-vinyl]-4H-benzo[1,4]oxazin-3-one | ¹H NMR (400 MHz,DMSO-_{d6}) δ 10.80 (s, 1 H),7.61 (d, J = 8.0 Hz, 1 H),7.25 (d, J = 16.4 Hz, 1 H),7.18 (dd, J = 8.0 Hz, 1.6Hz, 1 H), 7.13-7.08 (m, 3 H),7.04 (d, J = 16.8 Hz, 1 H),6.96 (d, J = 8.4 Hz, 1 H),4.60 (s, 2 H), 2.31 (s, 3 H).MS: (ES⁺) 284 m/z (M + 1)⁺C₁₇H₁₄FNO₂ requires 284 |
| 45 | 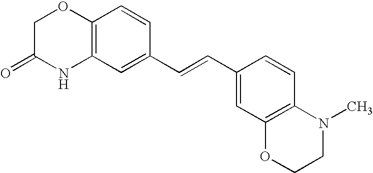 6-[2-(4-Methyl-3,4-dihydro-2H-benzo[1,4]oxazin-7-yl)-vinyl]-4H-benzo[1,4]oxazin-2-one | ¹H NMR (400 MHz,DMSO-_{d6}) δ 10.80 (s, 1 H),7.13-7.08 (m, 1 H), 7.00-6.96(m, 2 H), 6.94-6.89 (m,2 H), 6.85 (d, J = 9.6 Hz,2 H), 6.66 (d, J = 4.4 Hz,1 H), 4.57 (s, 2 H), 4.23 (t,J = 4.0 Hz, 2 H), 3.25 (t, J =4.4 Hz, 2 H), 2.85 (s, 3 H).MS: (ES⁺) 323 m/z (M + 1)⁺C₁₉H₁₈N₂O₃ requires 323 |
| 46 | 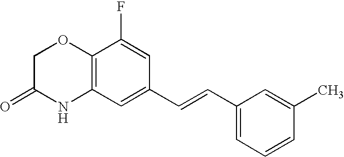 8-Fluoro-6-(2-m-tolyl-vinyl)-4H-benzo[1,4]oxazin-3-one | ¹H NMR (400 MHz,DMSO-_{d6}) δ 11.00 (s, 1 H),7.42 (s, 1 H), 7.36 (d, J =8.0 Hz, 1 H), 7.28-7.24 (m,1 H), 7.22 (dd, J = 12.0 Hz,2.0 Hz, 1 H), 7.17-7.04 (m,3 H), 6.87 (s, 1 H), 4.68 (s,2 H), 2.32 (s, 3 H). MS:(ES⁺) 284 m/z (M + 1)⁺C₁₄H₁₄FNO₂ requires 284 |
| 47 | 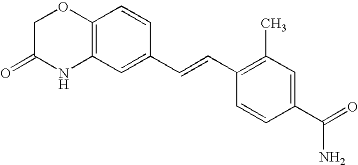 3-Methyl-4-[2-(3-oxo-3,4-dihydro-2H-benzo[1,4]oxazin-6-yl)-vinyl]-benzamide | . ¹H NMR (400 MHz,DMSO-_{d6}) δ 10.80 (s, 1 H),7.93 (s, 1 H), 7.78-7.68 (m,3 H), 7.30 (s, 1 H), 7.25 (dd,J = 8.4 Hz, 2.0 Hz, 1 H),7.20 (d, J = 4.0 Hz, 2 H),7.13 (d, J = 2.0 Hz, 1 H),6.97 (d, J = 8.0 Hz, 1 H),4.60 (s, 2 H), 2.43 (s, 3 H).MS: (ES⁺) 309 m/z (M + 1)⁺C₁₈H₁₆N₂O₃ requires 309 |
| 48 | 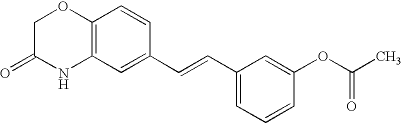 Acetic acid 3-[2-(3-oxo-3,4-dihydro-2H-benzo[1,4]oxazin-6-yl)-vinyl]-phenyl ester | ¹H NMR (400 MHz,CDCl₃) δ 10.80 (s, 1 H),7.46 (d, J = 7.6 Hz, 1 H),7.41-7.36 (m, 2 H), 7.25-7.18(m, 2 H), 7.07 (d, J =2.0 Hz, 1 H), 7.05-6.99 (m,2 H), 6.96 (d, J = 8.4 Hz,1 H), 4.60 (s, 2 H), 2.28 (s,3 H). MS: (ES⁺) 310 m/z(M + 1)⁺C₁₈H₁₅NO₄ requires 310 |
| 49 | 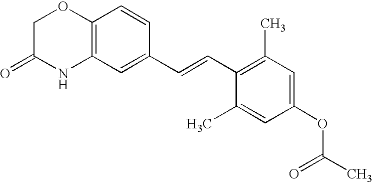 Acetic acid 3,5-dimethyl-4-[2-(3-oxo-3,4-dihydro-2H-benzo[1,4]oxazin-6-yl)-vinyl]-phenyl ester | ¹H NMR (400 MHz,CDCl₃) δ 10.80 (s, 1 H),7.28 (dd, J = 8.4 Hz, 2.0Hz, 2 H), 7.80 (d, J = 1.6Hz, 1 H), 7.00-6.94 (m, 2 H),6.84 (s, 2 H), 6.60 (d,J = 16.8 Hz, 1 H), 4.58 (s, 2 H),2.30 (s, 6 H), 2.25 (s, 3 H).MS: (ES⁺) 338 m/z (M + 1)⁺C₂₀H₁₉NO₄ requires 338 |
| 50 | 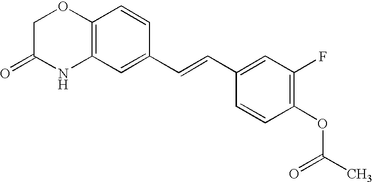 Acetic acid 2-fluoro-4-[2-(3-oxo-3,4-dihydro-2H-benzo[1,4]oxazin-6-yl)-vinyl]-phenyl ester | ¹H NMR (400 MHz,CDCl₃) δ 10.80 (s, 1 H),7.66 (dd, J = 12.0 Hz, 2.0Hz, 1 H), 7.42 (dd, J = 8.0Hz, 1.6 Hz, 1 H), 7.30-7.22(m, 2 H), 7.20 (dd, J = 8.4Hz, 1.6 Hz, 1 H), 7.07 (d,J = 2.0 Hz, 1 H), 7.02 (d,J = 16.4 Hz, 1 H), 6.97 (d, J =8.4 Hz, 1 H), 4.60 (s, 2 H),2.32 (s, 3 H). MS: (ES⁺)328 m/z (M + 1)⁺C₁₈H₁₄FNO₄ requires 328 |
| 51 | 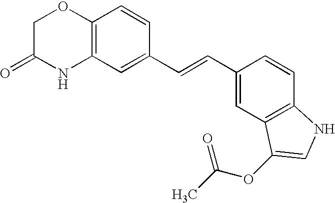 Acetic acid 5-[2-(3-oxo-3,4-dihydro-2H-benzo[1,4]oxazin-6-yl)-vinyl]-1H-indol3-yl ester | ¹H NMR (400 MHz,CDCl₃) δ 11.20 (s, 1 H),10.80 (s, 1 H), 7.56 (s, 1 H),7.43 (d, J = 8.0 Hz, 1 H),7.36-7.32 (m, 2 H), 7.18(dd, J = 8.0 Hz, 1.2 Hz,1 H), 7.09 (d, J = 2.8 Hz,2 H), 7.06 (d, J = 1.6 Hz,1 H), 6.94 (d, J = 8.4 Hz,1 H), 4.58 (s, 2 H), 2.34 (s,3 H). MS: (ES⁺) 349 m/z(M + 1)⁺C₂₀H₁₆N₂O₄ requires 349 |
| 52 | 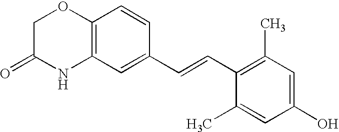 6-[2-(4-Hydroxy-2,6-dimethyl-phenyl)-vinyl]-4H-benzo[1,4]oxazin-3-one | . ¹H NMR (400 MHz,DMSO-_{d6}) δ 10.70 (s, 1 H),9.22 (s, 1 H), 7.12 (dd, J =8.4 Hz, 2.0 Hz, 1 H), 7.04(d, J = 2.0 Hz, 1 H), 6.94 (s,1 H), 6.91 (d, J = 7.2 Hz,1 H), 6.60-6.45 (m, 3 H),4.54 (s, 2 H), 2.24 (s, 6 H).MS: (ES⁺) 296 m/z (M + 1)⁺C₁₈H₁₇NO₃ requires 296 |
| 53 | 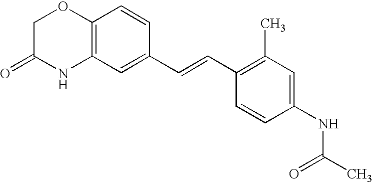 N-{3-Methyl-4-[2-(3-oxo-3,4-dihydro-2H-benzo[1,4]oxazin-6-yl)-vinyl]-phenyl}-acetamide | ¹H NMR (400 MHz,DMSO_{d6}) δ 10.72 (s, 1 H),9.92 (s, 1 H), 7.60 (d, J =9.6 Hz, 1 H), 7.44-7.42 (m,1 H), 7.42 (s, 1 H), 7.18 (dd,J = 8.4 Hz, 2.0 Hz, 1 H),7.14 (d, J = 15.6 Hz, 1 H),7.08 (d, J = 2.0 Hz, 1 H),7.00-6.92 (m, 2 H), 4.59 (s,2 H), 2.35 (s, 3 H), 2.04 (s,3 H). MS: (ES⁺) 323 m/z(M + 1)⁺C₁₉H₁₈N₂O₃ requires 323 |
| 54 | 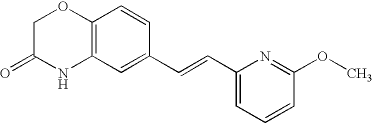 6-[2-(6-Methoxy-pyridin-2-yl)-vinyl]-4H-benzo[1,4]oxazin-3-one | ¹H NMR (400 MHz,DMSO-_{d6}) δ 10.80 (s, 1 H),7.66 (dd, J = 8.4 Hz, 7.6Hz, 1 H), 7.57 (d, J = 15.6Hz, 1 H), 7.23 (dd, J = 4.4Hz, 2.0 Hz, 1 H), 7.11 (d,J = 2.0 Hz, 1 H), 7.08 (d, J =7.2 Hz, 1 H), 7.02 (d, J =16.0 Hz, 1 H), 6.96 (d, J =8.4 Hz, 1 H), 6.67 (d, J =8.0 Hz, 1 H), 4.60 (s, 2 H),3.92 (s, 3 H). MS: (ES⁺)283 m/z (M + 1)⁺C₁₆H₁₄N₂O₃ requires 283 |
| 55 | 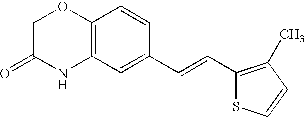 6-[2-(3-Methyl-thiophen-2-yl)-vinyl]-4H-benzo[1,4]oxazin-3-one | ¹H NMR (400 MHz,DMSO-_{d6}) δ 10.70 (s, 1 H),7.34 (d, J = 5.2 Hz, 1 H),7.22-7.15 (m, 2 H), 7.04 (d,J = 1.6 Hz, 1 H), 6.94 (d, J =8.0 Hz, 1 H), 6.90 (d, J =5.2 Hz, 1 H), 6.74 (d, J =16.0 Hz, 1 H), 4.58 (s, 2 H),2.28 (s, 3 H). MS: (ES⁺)272 m/z (M + 1)⁺C₁₅H₁₃NO₂S requires 272 |
| 56 | 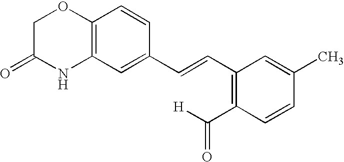 4-Methyl-2-[2-(3-oxo-3,4-dihydro-2H-benzo[1,4]oxazin-6-yl)-vinyl]-benzaldehyde | . ¹H NMR (400 MHz,DMSO_{d6}) δ 10.82 (s, 1 H),10.24 (s, 1 H), 8.01 (d, J =16.0 Hz, 1 H), 7.80-7.74 (m,2 H), 7.34-7.30 (m, 1 H),7.26-7.20 (m, 2 H), 7.15 (d,J = 2.0 Hz, 1 H), 7.00-6.96(m, 1 H), 4.60 (s, 2 H), 2.42(s, 3 H). MS: (ES⁺) 294m/z (M + 1)⁺C₁₈H₁₅NO₃ requires 294 |
| 57 | 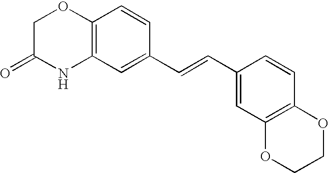 6-[2-(2,3-Dihydro-benzo[1,4]dioxin-6-yl)-vinyl]-4H-benzo[1,4]oxazin-3-one | ¹H NMR (400 MHz,DMSO-_{d6}) δ 10.75 (s, 1 H),7.14 (dd, J = 8.4 Hz, 2.0Hz, 1 H), 7.10 (d, J = 2.0Hz, 1 H), 7.06-6.98 (m, 3 H),6.94-6.86 (m, 2 H), 6.83 (d,J = 8.4 Hz, 1 H), 4.58 (s,2 H), 4.24 (s, 4 H). MS:(ES⁺) 310 m/z (M + 1)⁺C₁₈H₁₅NO₄ requires 310 |
| 58 | 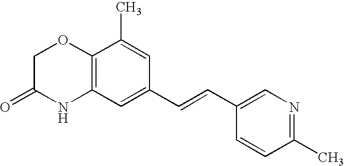 8-Methyl-6-[2-(6-methyl-pyridin-3-yl)-vinyl]-4H-benzo[1,4]oxazin-3-one | ¹H NMR (400 MHz,DMSO-_{d6}) δ 10.70 (s, 1 H),8.78 (s, 1 H), 8.30 (d, J =4.8 Hz, 1 H), 7.22 (d, J =4.8 Hz, 1 H), 7.17 (s, 1 H),7.14 (s, 2 H), 6.94 (d, J =1.6 Hz, 1 H), 4.60 (s, 2 H),2.40 (s, 3 H), 2.20 (s, 3 H).(ES⁺) 281 m/z (M + 1)⁺C₁₇H₁₆N₂O₂ requires 281 |
| 59 | 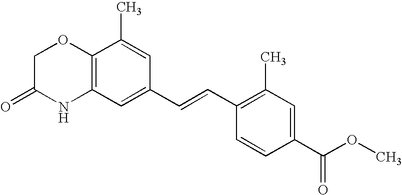 3-Methyl-2-[2-(8-methyl-3-oxo-3,4-dihydro-2H-benzo[1,4]oxazin-6-yl)-vinyl]-benzoic acid methyl ester | ¹H NMR (400 MHz,DMSO_{d6}) δ 10.70 (s, 1 H),7.84-7.75 (m, 3 H), 7.23-7.18(m, 3 H), 6.97 (s, 1 H),4 62 (s, 2 H), 3.84 (s, 3 H),2.45 (s, 3 H), 2.20 (s, 3 H).MS: (ES⁺) 338 m/z (M + 1)⁺C₂₀H₁₉NO₄ requires 338 |
| 60 | 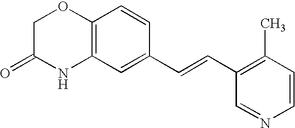 8-Methyl-6-[2-(4-methyl-pyridin-3-yl-vinyl]-4H-benzo[1,4]oxazin-3-one | . ¹H NMR (400 MHz,DMSO-_{d6}) δ 10.70 (s, 1 H),8.60 (d, J = 2.0 Hz, 1 H),7.92 (dd, J = 8.0 Hz, 2.0Hz, 1 H), 7.24 (d, J = 8.0Hz, 1 H), 7.20 (d, J = 16.4Hz, 1 H), 7.12 (d, J = 1.6Hz, 1 H), 7.00 (d, J = 16.4Hz, 1 H), 6.90 (d, J = 1.6Hz, 1 H), 4.60 (s, 2 H), 2.46(s, 3 H), 2.18 (s, 3 H). MS:(ES⁺) 281 m/z (M + 1)⁺C₁₇H₁₆N₂O₂ requires 281 |
| 61 | 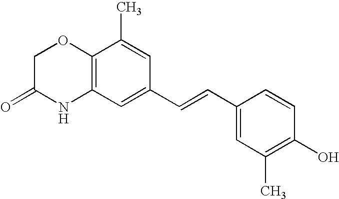 6-[2-(4-Hydroxy-3-methyl-phenyl)-vinyl-8-methyl-]-4H-benzo[1,4]oxazin-3-one | ¹H NMR (400 MHz,DMSO-_{d6}) δ 10.70 (s, 1 H),9.40 (s, 1 H), 7.30 (s, 1 H),7.18 (dd, J = 8.0 Hz, 1.6Hz, 1 H), 7.02 (d, J = 1.6Hz, 1 H), 6.88 (s, 2 H), 6.84(s, 1 H), 6.74 (d, J = 8.4 Hz,1 H), 4.58 (s, 2 H), 2.18 (s,3 H), 2.14 (s, 3 H). MS:(ES⁺) 296 m/z (M + 1)⁺C₁₈H₁₇NO₃ requires 296 |
| 62 | 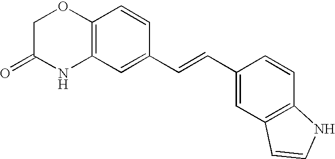 6-[2-(1H-Indol-5-yl)-vinyl]-4H-benzo[1,4]oxazin-3-one | . ¹H NMR (400 MHz,DMSO-_{d6}) δ 11.20 (s, 1 H),10.70 (s, 1 H), 7.70 (s, 1 H),7.40-7.37 (m, 2 H), 7.35-7.31(m, 1 H), 7.16 (dd, J =7.31 (m, 1H), 7.16 (dd, J =8.4 Hz, 1.6 Hz, 1 H), 7.08-7.057.05 (m, 3 H), 6.94 (d, J =8.0 Hz, 1 H), 6.42 (d, J =2.0 Hz, 1 H), 4.58 (s, 2 H).MS: (ES⁺) 291 m/z (M + 1)⁺C₁₈H₁₄N₂O₂ requires 291 |
| 63 | 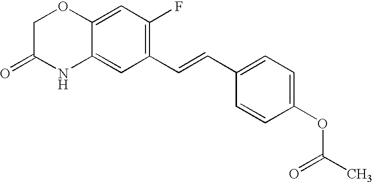 Acetic acid 4-[2-(7-fluoro-3-oxo-3,4-dihydro-2H-benzo[1,4]oxazin-6-yl)-vinyl]-phenyl | ¹H NMR (400 MHz,DMSO-_{d6}) δ, 10.80 (s, 1 H),7.65 (d, J = 8.4 Hz, 2 H),7.21-7.11 (m, 4 H), 7.05 (d,J = 16.4 Hz, 1 H), 6.95 (d,J = 11.2 Hz, 1 H), 4.62 (s,2 H), 2.28 (s, 3 H). MS:(ES⁺) 328 m/z (M + 1)⁺C₁₈H₁₄FNO₄ requires 328 |
| 64 | 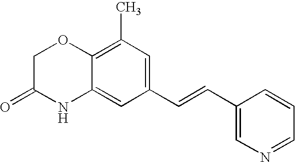 8-Methyl-6-(2-pyridin-3-yl-vinyl)-4H-benzo[1,4]oxazin-3-one | ¹H NMR (400 MHz,DMSO-_{d6}) δ^{~}165 (s, 1 H),8.64 (s, 1 H), 8.32 (d, J =4.0 Hz, 1 H), 7.92 (d, J =8.0 Hz, 1 H), 7.35-7.36 (m,1 H), 7.26-7.29 (m, 1 H),7.17 (d, J = 16.4 Hz, 1 H),7.03 (s, 1 H), 6.93 (d, J =16.4 Hz, 1 H), 6.82 (d, J =1.2 Hz, 1 H), 4.50 (s, 2 H),2.58 (s, 3 H). MS: (ES⁺) 267m/z (M + 1)⁺C₁₆H₁₅N₂O₂ requires 267 |
| 65 | 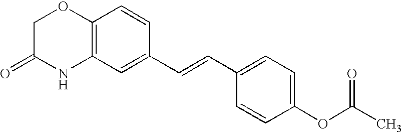 acetic acid 4-[2-(3-oxo-3,4-dihydro-2H-benzo[1,4]oxazin-6-yl)-vinyl]-phenyl ester | ¹H NMR (400 MHz,DMSO-_{d6}) δ 10.72 (s, 1 H),7.55 (d, J = 8.4 Hz, 2 H),7.11-7.14 (m, 1 H), 7.07 (d,J = 5.6 Hz, 2 H, 7.04 (s, 1 H),7.00 (d, J = 1.6 Hz, 1 H),6.98 (s, 1 H), 6.89 (d, J =8.4 Hz, 1 H), 4.52 (s, 2 H),2.21 (s, 3 H). MS: (ES⁺) 310m/z (M + 1)⁺C₁₈H₁₆NO₄ requires 310 |
| 66 | 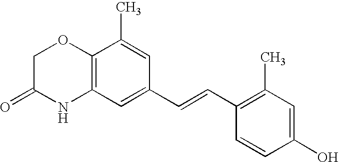 6-[2-(4-Hydroxy-2-methyl-phenyl)-vinyl]-8-methyl-4H-benzo[1,4]oxazin-3-one | . ¹H NMR (600 MHz,DMSO-_{d6}) δ 10.60 (s, 1 H),9.40 (s, 1 H), 7.47 (d, J =5.6 Hz, 1 H), 7.08 (d, J =10.8 Hz, 1 H), 7.04 (s, 1 H),6.88-6.90 (m, 1 H), 6.79 (d,J = 10.8 Hz, 1 H), 6.02-6.58(m, 2 H), 4.58 (s, 2 H), 2.30(s, 3 H), 2.18 (s, 3 H). MS:(ES⁺) 296 m/z (M + 1)⁺C₁₈H₁₈NO₃ requires 296 |
| 67 | 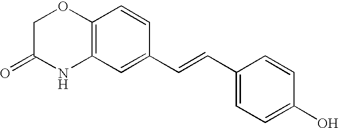 6-[2-(4-Hydroxy-phenyl)-vinyl]-4H-benzo[1,4]oxazin-3-one | ¹H NMR (400 MHz,DMSO-_{d6}) δ 10.53 (s, 1 H),9.35 (s, 1 H), 7.18 (d, J =8.4 Hz, 2 H), 6.88-6.92 (m,1 H), 6.81 (d, J = 2 Hz, 1 H),6.70-6.72 (m, 3 H), 6.54 (d,J = 8.4 Hz, 2 H), 4.36 (s,2 H). MS: (ES⁺) 268 m/z(M + 1)⁺C₁₆H₁₄NO₃ requires 268 |
| 68 | 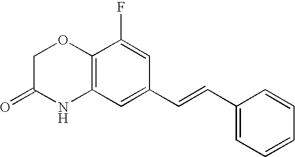 8-Fluoro-6-styryl-4H-benzo[1,4]oxazin-3-one | ¹H NMR (400 MHz,DMSO-_{d6}) δ 10.26 (s, 1 H),7.79 (d, J = 7.6 Hz, 2 H),7.59 (t, J = 7.6 Hz, 2 H),7.41-7.50 (m, 2 H), 7.35(dd, J = 10, 16.4 Hz, 2 H),7.09 (s, 1 H), 4.89 (s, 2 H),MS: (ES⁺) 270 m/z (M + 1)⁺C₁₆H₁₃FNO₂ requires 270 |
| 69 | 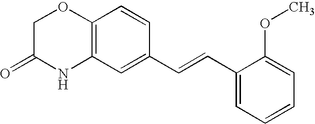 6-[2-(2-Methoxy-phenyl)-vinyl]-4H-benzo[1,4]oxazin-3-one | ¹H NMR (400 MHz,DMSO-_{d6}) δ 10.94 (s, 1 H),7.89 (dd, J = 1.6, 8 Hz,1 H), 7.50-7.15 (m, 8 H),4.81 (s, 2 H), 4.01 (s, 3 H);MS: (ES⁺) 294 m/z (M + 1)⁺C₁₈H₁₆NO₃ requires 294 |
| 70 | 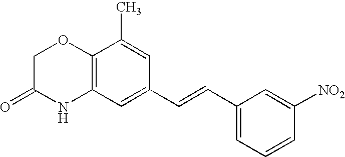 8-Methyl-6-[2-(3-nitro-phenyl)-vinyl]-4H-benzo[1,4]oxazin-3-one | ¹H NMR (400 MHz,DMSO-_{d6}) δ 11.02 (s, 1 H),8.64 (s, 1 H), 8.29 (t, J = 8.8Hz, 2 H), 7.87 (t, J = 8.0Hz, 1 H), 7.62 (d, J = 16.4Hz, 2 H), 7.44-7.40 (m, 2 H),7.17 (s, 1 H), 4.84 (s, 2 H),2.40 (s, 3 H); MS: (ES⁺)311 m/z (M + 1)⁺C₁₇H₁₅N₂O₄ requires 311 |
| 71 | 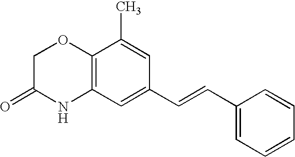 8-Methyl-6-styryl-4H-benzo[1,4]oxazin-3-one | MS: (ES⁺) 266 m/z (M + 1)⁺C₁₇H₁₆NO₂ requires 266 |
| 72 | 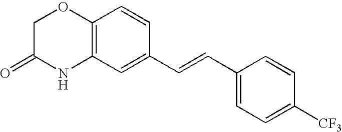 6-[2-(4-Trifluoromethyl-phenyl)-vinyl]-4H-benzo[1,4]oxazin-3-one | MS: (ES⁺) 320 m/z (M + 1)⁺C₁₇H₁₂F₃NO₂ requires 320 |

The following examples of table 2 were synthesized according to reference 2

TABLE 2

| | | Physical Data |
|---|---|---|
| | | ¹H NMR 400 MHz |
| Compound | | (CDCl₃ or DMSO) and/or |
| Number | Structure | MS (m/z) (M + 1)⁺ |
| 73 | 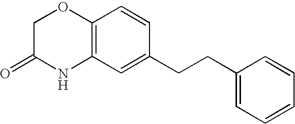 6-Phenethyl-4H-benzo[1,4]oxazin-3-one | ¹H NMR (400MHz, DMSO-_{d6}) δ10.65 (s, 1 H), 7.16-7.29(m, 5 H), 6.84 (d, J = 8 Hz,1 H), 6.74-6.87 (m, 2 H),4.52 (s, 2 H), 2.75-2.86 (m,4 H). MS: (ES⁺) 254 m/z(M + 1)⁺C₁₆H₁₆NO₂ requires 254 |
| 74 | 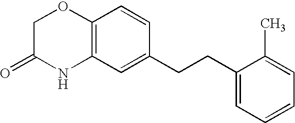 6-(2-o-tolyl-ethyl)-4H-benzo[1,4]oxazin-3-one | . ¹H NMR (400 MHz,DMSO-_{d6}) δ 10.65 (s, 1 H),7.08-7.18 (m, 4 H), 6.85-6.88,(m, 1 H), 6.76-6.81(m, 3 H), 4.54 (s, 2 H),2.70-2.82 (m, 4 H). MS:(ES⁺) 268 m/z (M + 1)⁺C₁₇H₁₈NO₂ requires 268 |
| 75 | 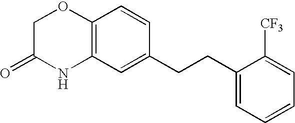 6-[2-(2-Trifluoromethyl-phenyl)-ethyl]-4H-benzo[1,4]oxazin-3-one | ¹H NMR (400 MHz,DMSO-_{d6}) δ 10.76 (s, 1 H),7.80 (d, J = 8 Hz, 1 H),7.73 (t, J = 7.6 Hz, 1 H),7.64 (d, J = 7.6 Hz, 1 H),7.53 (t, J = 8 Hz, 1 H),6.98, (d, J = 8 Hz, 1 H),6.88-6.92 (m, 2 H), 4.63 (s,2 H), 2.85-3.09 (m, 4 H).MS: (ES⁺) 322 m/z(M + 1)⁺C₁₇H₁₅F₃NO₂ requires 322 |
| 76 | 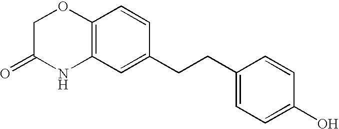 6-[2-(4-Hydroxy-phenyl)-ethyl]-4H-benzo[1,4]oxazin-3-one | ¹H NMR (400 MHz,DMSO-_{d6}) δ 10.64 (s, 1 H),9.14 (s, 1 H), 6.99 (d, J = 8Hz, 2 H), 6.83 (d, J = 8Hz, 1 H), 6.71-6.76 (m,2 H), 6.63-6.67 (m, 2 H),4.52 (s, 2 H), 2.69-2.72 (m,4 H). MS: (ES⁺) 270 m/z(M + 1)⁺C₁₆H₁₆NO₃ requires 270 |
| 77 | 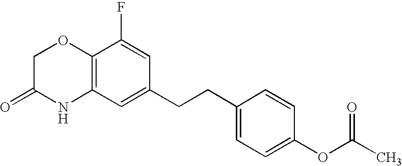 Acetic acid 4-[2-(8-fluoro-3-oxo-3,4-dihydro-2H-benzo[1,4]oxazin-6-yl)-ethyl]-phenyl ester | ¹H NMR (400 MHz,DMSO-_{d6}) δ 10.68 (s, 1 H),7.26 (d, J = 8.4 Hz, 2 H),7.02 (d, J = 8.4 Hz, 2 H),6.80 (dd, J = 1.6, 11.6 Hz,1 H), 6.57 (s, 2 H), 4.62 (s,2 H), 2.76-2.86 (m, 4 H),2.25 (s, 3 H). MS: (ES⁺)330 m/z (M + 1)⁺C₁₈H₁₇FNO₄ requires 330 |
| 78 | 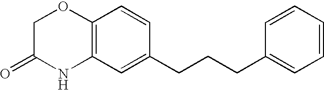 6-(3-Phenyl-propyl)-4H-benzo[1,4]oxazin-3-one | ¹H NMR (400 MHz,DMSO-_{d6}) δ 10.47 (s, 1 H),6.97-7.14 (m, 5 H), 6.68 (d,J = 8 Hz,1 H), 6.54-6.68(m, 2 H), 4.62 (s, 2 H),2.34-2.44 (m, 4 H), 1.63-1.67(m, 2 H) 0.94-0.96 (m,2 H). MS: (ES⁺) 268 m/z(M + 1)⁺C₁₇H₁₈NO₂ requires 268 |
| 79 | 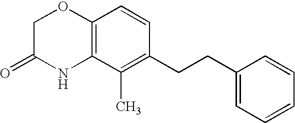 5-Methyl-6-phenethyl-4H-benzo[1,4]oxazin-3-one | ¹H NMR (400 MHz,DMSO-_{d6}) δ 10.18 (s, 1 H),7.19-7.31 (m, 5 H), 6.73-6.81(m, 2 H), 4.48 (s, 2 H),2.74-2.81 (m, 4 H), 2.18 (s,3 H). MS: (ES⁺) 268 m/z(M + 1)⁺C₁₇H₁₈NO₂ requires 238 |
| 80 | 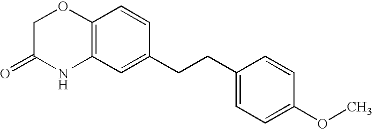 6-[2-(4-Methoxy-phenyl)-ethyl]-4H-benzo[1,4]oxazin-3-one | MS: (ES⁺) 284 m/z(M + 1)⁺C₁₇H₁₇NO₃ requires 284 |
| 81 | 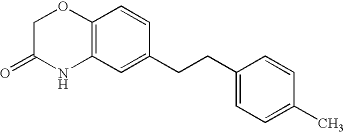 6-(2-p-Tolyl-ethyl)-4H-benzo[1,4]oxazin-3-one | MS: (ES⁺) 268 m/z (M + 1)⁺C₁₇H₁₇NO₂ requires 268 |
| 82 | 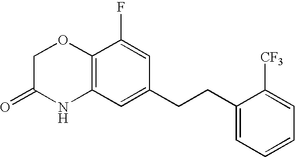 8-Fluoro-6-[2-(2-trifluoromethyl-phenyl)-ethyl]-4H-benzo[1,4]oxazin-3-one | MS: (ES⁺) 338 m/z(M + 1)⁺C₁₇H₁₁F₄NO₂ requires 338 |
| 83 | 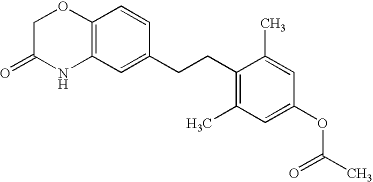 Acetic acid 3,5-dimethyl-4-[2-(3-oxo-3,4-dihydro-2H-benzo[1,4]oxazin-6-yl)-ethyl]-phenyl ester | MS: (ES⁺) 340 m/z (M + 1)⁺C₂₀H₂₁NO₄ requires 340 |
| 84 | 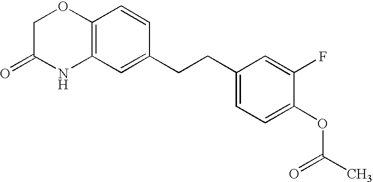 Acetic acid 2-fluoro-4-[2-(3-oxo-3,4-dihydro-2H-benzo[1,4]oxazin-6-yl)-ethyl]-phenyl ester | MS: (ES⁺) 330 m/z (M + 1)⁺C₁₈H₁₆FNO₄ requires 330 |
| 85 | 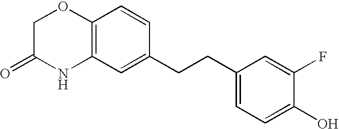 6-[2-(3-Fluoro-4-hydroxy-phenyl)-ethyl]-4H-benzo[1,4]oxazin-3-one | MS: (ES⁺) 288 m/z (M + 1)⁺C₁₆H₁₄FNO₃ requires 288 |
| 86 | 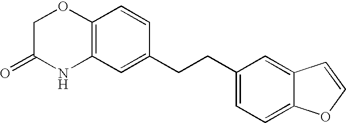 6-(2-Benzofuran-5-yl-ethyl)-4H-benzo[1,4]oxazin-3-one | MS: (ES⁺) 294 m/z (M + 1)⁺C₁₈H₁₆NO₃ requires 294 |
| 87 | 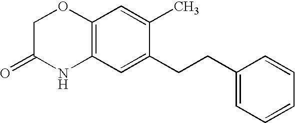 7-Methyl-6-phenethyl-4H-benzo[1,4]oxazin-3-one | ¹H NMR (400 MHz,DMSO-_{d6}) δ 10.91 (s, 1 H),7.65-7.50 (m, 5 H),7.09 (s, 1 H), 7.04 (s, 1 H),4.84 (s, 2 H), 3.09 (s, 3 H),2.85 (m, 4 H); MS: (ES⁺)268 m/z (M + 1)⁺C₁₇H₁₈NO₂ requires 268 |
| 88 | 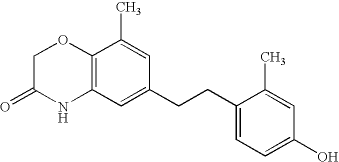 6-[2-(4-Hydroxy-2-methyl-phenyl)-ethyl]-8-methyl-4H-benzo[1,4]oxazin-3-one | ¹H NMR (400 MHz,DMSO-_{d6}) δ 10.54 (s, 1 H),9.01 (s, 1 H), 6.93 (d, J =5.6 Hz, 1 H), 6.67 (s, 1 H),6.58 (s, 1 H), 6.55 (s, 1 H),6.50 (dd, J = 2.0, 5.6 Hz,1 H), 4.52 (s, 2 H), 2.17 (s,3 H), 2.13 (s, 3 H); MS:(ES⁺) 298 m/z (M + 1)⁺C₁₈H₂₀NO₃ requires 298 |
| 89 | 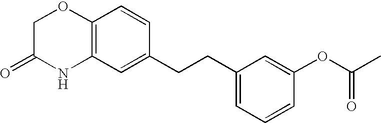 Acetic acid 3-[2-(3-oxo-3,4-dihydro-2H-benzo[1,4]oxazin-6-yl)-ethyl]-phenyl ester | MS: (ES⁺) 312 m/z (M + 1)⁺C₁₈H₁₈NO₄ requires 312 |
| 90 | 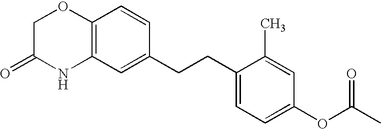 Acetic acid 3-methyl-4-[2-(3-oxo-3,4-dihydro-2H-benzo[1,4]oxazin-6-yl)-ethyl]-phenyl ester | MS: (ES⁺) 326 m/z (M + 1)⁺C₁₉H₂₀NO₄ requires 326 |
| 91 | 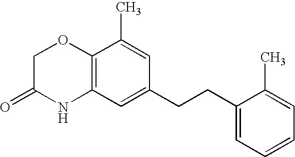 8-Methyl-6-(2-o-tolyl-ethyl)-4H-benzo[1,4]oxazin-3-one | ¹H NMR (400 MHz,DMSO-_{d6}) δ 10.56 (s, 1 H),7.17-7.06 (m, 4 H), 6.69 (s,1 H), 6.61 (s, 1 H), 4.53 (s,2 H), 2.31-2.26 (m, 2 H),2.20-2.16 (m, 2 H), 2.27 (s,3 H), 2.13 (s, 3 H); MS:(ES⁺) 282 m/z (M + 1)⁺C₁₈H₂₀NO₂ requires 282 |
| 92 | 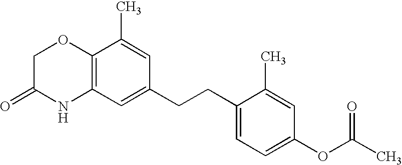 Acetic acid 3-methyl-4-[2-(8-methyl-3-oxo-3,4-dihydro-2H-benzo[1,4]oxazin-6-yl)-ethyl]-phenyl ester | ¹H NMR (400 MHz,DMSO-_{d6}) δ 10.56 (s, 1 H),7.18 (d, J = 7.8 Hz, 1 H),6.91 (br s, 1 H), 6.86 (dd, J =2.4, 8.4 Hz, 1 H), 6.69 (s,1 H), 6.62 (s, 1 H), 4.53 (s,2 H), 2.57-2.51 (m, 2 H),2.39-2.32 (m, 2 H), 2.27 (s,3 H), 2.24 (s, 3 H), 2.13 (s,3 H); MS: (ES⁺) 340 m/z(M + 1)⁺C₂₀H₂₂NO₄ requires 340 |
| 93 | 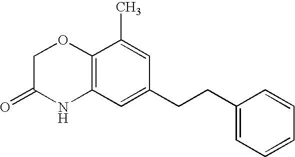 8-Methyl-6-phenethyl-4H-benzo[1,4]oxazin-3-one | ¹H NMR (400 MHz,DMSO-_{d6}) δ 10.64 (s, 1 H),7.30-7.15 (m, 5 H),6.68 (s, 1 H), 6.58 (d, J =2.0 Hz, 1 H), 2.85-2.70 (m,4 H), 2.19 (s, 3 H); MS:(ES⁺) 268 m/z (M + 1)⁺C₁₇H₁₈NO₂ requires 268 |
| 94 | 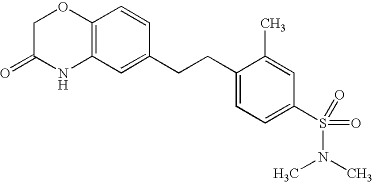 3,N,N-Trimethyl-4-[2-(3-oxo-3,4-dihydro-2H-benzo[1,4]oxazin-6-yl)-ethyl]-benzenesulfonamide | . ¹H NMR (400 MHz,DMSO-_{d6}) δ 10.61 (s, 1 H),7.48-7.35 (m, 3 H), 6.81 (d,J = 8.4 Hz, 1 H), 6.75-6.69(m, 2 H), 4.48 (s, 2 H),2.86-2.82 (m, 2 H), 2.74-2.70(m, 2 H), 2.53 (s, 6 H),2.31 (s, 3 H); MS: (ES⁺)375 m/z (M + 1)⁺C₁₉H₂₃N₂O₄S requires 375 |
| 95 | 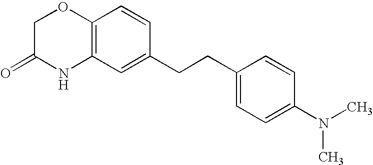 6-[2-(4-Dimethylamino-phenyl)-ethyl]-4H-benzo[1,4]oxazin-3-one | ¹H NMR (400 MHz,DMSO-_{d6}) δ 10.83 (s, 1 H),7.36 (d, J = 6.8 Hz, 2 H),7.19 (br s, 2 H), 7.02-6.90(m, 3 H), 4.69 (s, 2 H), 3.15(s, 6 H), 2.93 (s, 3 H); MS:(ES⁺) 297 m/z (M + 1)⁺C₁₈H₂₁N₂O₂ requires 297 |
| 96 | 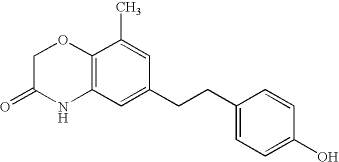 6-[2-(4-Hydroxy-phenyl)-ethyl]-8-methyl-4H-benzo[1,4]oxazin-3-one | ¹H NMR (400 MHz,DMSO-_{d6}) δ 10.57 (s, 1 H),9.14 (s, 1 H), 6.99 (d, J =8.4 Hz, 2 H), 6.66-6.40 (m,3 H), 6.55 (d, J = 2.0 Hz,1 H), 4.52 (s, 2 H), 2.67 (m,4 H), 2.16 (s, 3 H), MS:(ES⁺) 284 m/z (M + 1)⁺C₁₇H₁₈NO₃ requires 284 |
| 97 | 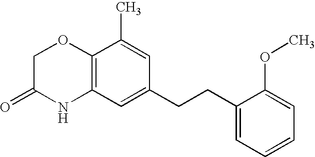 6-[2-(2-Methoxy-phenyl)-ethyl]-8-methyl-4H-benzo[1,4]oxazin-3-one | . ¹H NMR (400 MHz,DMSO-_{d6}) δ 10.57 (s, 1 H),7.21-7.11 (m, 2 H), 6.96 (d,J = 7.6 Hz, 1 H), 6.86 (t, J =7.6 Hz, 1 H), 6.66 (s,1 H), 6.59 (s, 1 H), 4.53 (s,2 H), 3.80 (s, 3 H), 2.78-2.65(m, 4 H), 2.13 (s, 3 H);MS: (ES⁺) 298 m/z(M + 1)⁺C₁₈H₂₀NO₃ requires 298 |
| 98 | 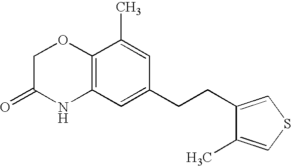 8-Methyl-6-[2-(4-methyl-thiophen-3-yl)-ethyl]-4H-benzo[1,4]oxazin-3-one | ¹H NMR (400 MHz,DMSO-_{d6}) δ 10.49 (s, 1 H),7.05-7.01 (m, 2 H), 6.61 (s,1 H), 6.52 (s, 1 H), 4.45 (s,2 H), 2.70 (m, 4 H), 2.04(s, 6 H); MS: (ES⁺) 288m/z (M + 1)⁺C₁₆H₁₈NO₂S requires 288 |
| 99 | 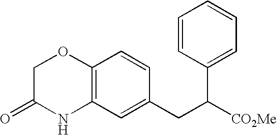 3-(3-Oxo-3,4-dihydro-2H-benzo[1,4]oxazin-6-yl)-2-phenyl-propionic acid methyl ester | ¹H NMR (400 MHz,DMSO-_{d6}) δ 10.65 (s, 1 H),7.32-7.24 (m, 5 H), 6.80 (d,J = 8.4 Hz, 1 H), 6.74-6.68(m, 2 H), 4.51 (s, 2 H), 3.89(ab quartet, J = 6.4, 9.2Hz, 1 H), 3.25-3.18 (m,1 H), 2.91-2.85 (m, 1 H);MS: (ES⁺) 312 m/z(M + 1)⁺ requires 312 |
| 100 | 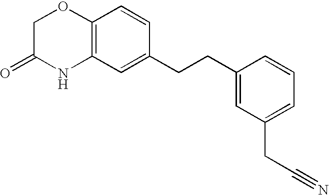 {3-[2-(3-Oxo-3,4-dihydro-2H-benzo[1,4]oxazin-6-yl)-ethyl]-phenyl}-acetonitrile | ¹H NMR (400 MHz,CDCl₃) δ 7.60 (s, 1 H),7.28 (s, 1 H), 7.15-7.10 (m,3 H), 6.88 (d, J = 8.0 Hz,1 H), 6.77 (dd, J = 9.2 Hz,3.0 Hz, 1 H), 6.54 (d, J =3.0 Hz, 1 H), 4.60 (s, 2 H),3.72 (s, 2 H), 2.92-2.82 (m,4 H). MS: (ES⁺) 293 m/z(M + 1)⁺C₁₈H₁₆N₂O₂ requires 293 |
| 101 | 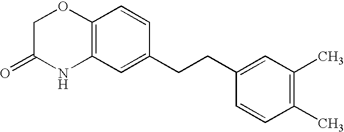 6-[2-(3,4-Dimethyl-phenyl)-ethyl]-4H-benzo[1,4]oxazin-3-one | ¹H NMR (400 MHz,DMSO-_{d6}) δ 10.47 (s, 1 H),7.15 (d, J = 8 Hz,1 H), 7.0 (s, 1 H), 6.9 (dd, J =7.3 Hz, 1.7 Hz, 1 H) 6.84(d, J = 8.1 Hz, 1 H), 6.70(dd, J = 8.0 Hz, 2.0 Hz,1 H), 6.73 (d, J = 2.0 Hz,1 H), 4.51 (s, 2 H), 2.73 (s,4 H), 2.16 (s, 3 H), 2.17 (s,3 H). MS: (ES⁺) 282 m/z(M + 1)⁺C₁₈H₁₉NO₂ requires 282 |
| 102 | 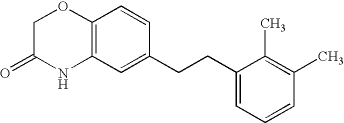 6-[2-(2,3-Dimethyl-phenyl)-ethyl]-4H-benzo[1,4]oxazin-3-one | . ¹H NMR (400 MHz,DMSO-_{d6}) δ 10.61 (s, 1 H),7.00-6.97 (m, 3 H), 6.86 (d,J = 8.0 Hz, 1 H), 6.78 (dd,J = 14.0 Hz, 2.0 Hz, 2 H),4.53 (s, 2 H), 2.83-2.77 (m,2 H), 2.70-2.64 (m, 2 H),2.23 (s, 3 H), 2.17 (s, 3 H).MS: (ES⁺) 282 m/z(M + 1)⁺C₁₈H₁₉NO₂ requires 282 |
| 103 | 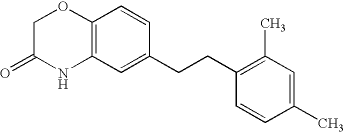 6-[2-(2,4-Dimethyl-phenyl)-ethyl]-4H-benzo[1,4]oxazin-3-one | ¹H NMR (400 MHz,DMSO-_{d6}) δ 10.61 (s, 1 H),6.93-6.88 (m, 3 H), 6.83-6.81(m, 1 H), 6.79-6.71(m, 2 H), 4.52 (s, 2 H),2.78-2.64 (m, 4 H), 2.26 (s,3 H), 2.22 (s, 3 H). MS:(ES⁺) 282 m/z (M + 1)⁺C₁₈H₁₉NO₂ requires 282 |
| 104 | 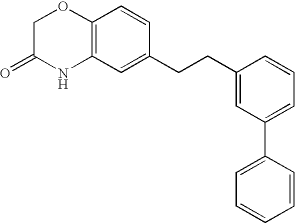 6-(2-Biphenyl-3-yl-ethyl)-4H-benzo[1,4]oxazin-3-one | . ¹H NMR (400 MHz,DMSO-_{d6}) δ 10.66 (s, 1 H),7.63 (d, J = 7.2 Hz, 2 H),7.50-7.43 (m, 4 H), 7.39-7.33(m, 2 H), 7.22 (d, J =7.6 Hz, 1 H), 6.86 (d, J =8.4 Hz, 1 H), 6.81 (dd, J =8.4 Hz, 2.0 Hz, 1 H), 6.76(d, J = 2.0 Hz, 1 H), 4.52(s, 2 H), 2.92-2.81 (m, 4 H).MS: (ES⁺) 330 m/z(M + 1)⁺C₂₂H₁₉NO₂ requires 330 |
| 105 | 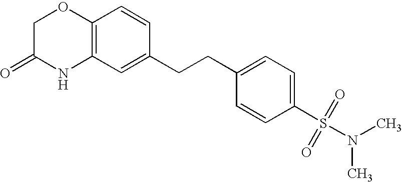 N,N-Dimethyl-4-[2-(3-oxo-3,4-dihydro-2H-benzo[1,4]oxazin-6-yl)-ethyl]-benzenesulfonamide | ¹H NMR (400 MHz,DMSO-_{d6}) δ 10.70 (s, 1 H),7.64 (d, J = 8.0 Hz,2 H), 7.48 (d, J = 8.0 Hz,2 H), 6.84 (d, J = 8.0 Hz,1 H), 6.78-6.68 (m, 2 H),4.52 (s, 2 H), 2.95-2.80 (m,4 H), 2.57 (s, 6 H). MS:(ES⁺) 361 m/z (M + 1)⁺C₁₈H₂₀N₂O₄S requires 361 |
| 106 | 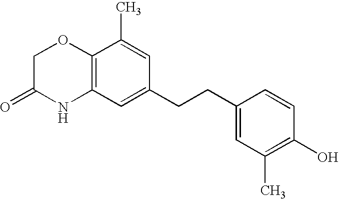 6-[2-(4-Hydroxy-3-methyl-phenyl)-ethyl]-8-methyl-4H-benzo[1,4]oxazin-3-one | ¹H NMR (400 MHz,DMSO-_{d6}) δ 10.60 (s, 1 H),9.00 (s, 1 H), 6.92 (s,1 H), 6.80 (dd, J = 8.0 Hz,2.0 Hz, 1 H), 6.68-6.64 (m,2 H), 6.56 (d, J = 1.6 Hz,1 H), 4.52 (s, 2 H), 2.65 (s,4 H), 2.13 (s, 3 H), 2.08 (s,3 H). MS: (ES⁺) 298 m/z(M + 1)⁺C₁₈H₁₉NO₃ requires 298 |
| 107 | 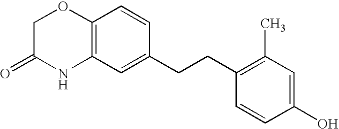 6-[2-(4-Hydroxy-2-methyl-phenyl)-ethyl]-4H-benzo[1,4]oxazin-3-one | ¹H NMR (400 MHz,DMSO-_{d6}) δ 10.56 (s, 1 H),8.96 (s, 1 H), 6.84 (d, J =8.4 Hz, 1 H), 8.78 (d, J =8.0 Hz, 1 H), 6.67-6.70 (m,2 H), 6.47 (d, J = 2.4 Hz,1 H), 6.45 (dd, J = 5.6 Hz,8.4 Hz, 1 H), 4.45 (s, 2 H),2.58 (s, 4 H), 2.10 (s, 3 H).MS: (ES⁺) 284 m/z(M + 1)⁺C₁₇H₁₈NO₃ requires 284 |
| 108 | 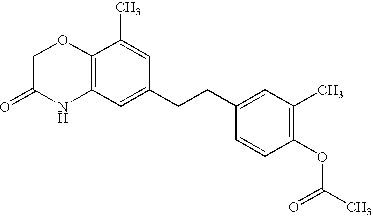 Acetic acid 2-methyl-4-[2-(8-methyl-3-oxo-3,4-dihydro-2H-benzo[1,4]oxazin-6-yl)-ethyl]-phenyl ester | ¹H NMR (400 MHz,DMSO-_{d6}) δ 10.60 (s, 1 H),7.16 (s, 1 H), 7.06 (dd, J =5.2 Hz, 1.2 Hz, 1 H), 6.94(d, J = 5.2 Hz, 1 H), 6.68(s, 1 H), 6.60 (s, 1 H), 4.52(s, 2 H), 2.80-2.70 (m, 4 H),2.28 (s, 3 H), 2.13 (s, 3 H),2.08 (s, 3 H). MS: (ES⁺)340 m/z (M + 1)⁺C₂₀H₂₂NO₄ requires 340 |
| 109 | 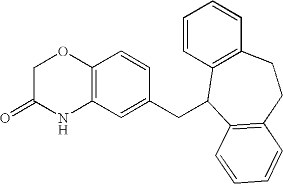 6-[10,11-dihydro-dibenzo[a,d]cyclohepten-5-ylmethyl]-4H-benzo[1,4]oxazin-3-one | ¹H NMR (400 MHz,CDCl₃) δ 7.77 (broad s,1 H), 7.05 (m, 3 H), 6.96(m, 2 H), 6.87 (m, 2 H),6.70 (dd, J = 8.4, 2.0 Hz,1 H), 6.50 (dt, J = 8.4,2.4 Hz, 1 H), 6.15 (d, J =2.0 Hz, 1 H), 4.49 (broad s,2 H), 4.03 (m, 2 H), 3.35(m, 2 H), 2.19 (m, 2 H),2.95 (m, 2 H), MS: (ES⁺)356 m/z (M + 1)⁺C₂₄H₂₁NO₂ requires 356 |
| 110 | 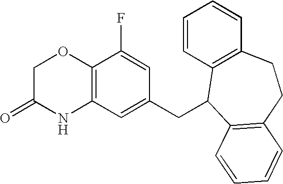 6-[10,11-dihydro-dibenzo[a,d]cyclohepten-5-ylidenemethyl]-8-fluoro-4H-benzo[1,4]oxazin-3-one | ¹H NMR (400 MHz,CDCl₃) δ 7.41 (broad s,2 H), 7.06 (m, 3 H), 6.96(m, 2 H), 6.81 (m, 2 H),6.37 (d, J = 11.2 Hz, 1 H),5.92 (broad s, 1 H), 4.55 (s,2 H), 4.02 (m, 1 H), 3.36(m, 2 H,), 3.17 (m, 2 H),2.95 (m, 2 H); MS: (ES⁺)374 m/z (M + 1)⁺C₂₄H₂₀FNO₂ requires 374 |
| 111 | 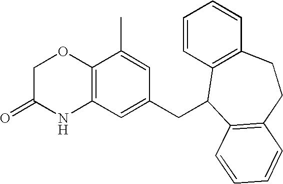 6-[10,11-dihydro-dibenzo[a,d]cyclohepten-5-ylidenemethyl]-8-methyl-4H-benzo[1,4]oxazin-3-one | ¹H NMR (400 MHz,CDCl₃) δ 7.59 (broad s,1 H), 7.17 (m, 8 H), 6.46 (s,1 H), 6.01 (s, 1 H), 4.57 (s,2 H), 4.06 (m, 1 H,), 3.45(m, 2 H), 3.25 (m, 2 H),3.02 (m, 2 H), 2.14 (s, 3 H);MS: (ES⁺) 368 m/z(M + 1)⁺C₂₅H₂₁NO₂ requires 368 |
| 112 | 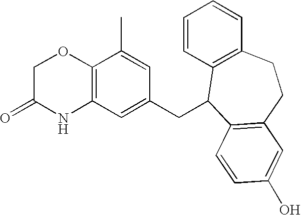 6-[10,11-dihydro-dibenzo[a,d]cyclohepten-4-hydroxy-5-ylidenemethyl]-8-methyl-4H-benzo[1,4]oxazin-3-one | MS: (ES⁺) 386 m/z(M + 1)⁺C₂₅H₂₃NO₃ requires 386 |
| 113 | 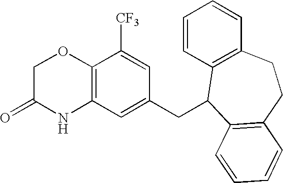 6-[10,11-dihydro-dibenzo[a,d]cyclohepten-5-ylidenemethyl]-8-trifluoromethyl-4H-benzo[1,4]oxazin-3-one | MS: (ES⁺) 424 m/z(M + 1)⁺C₂₅H₂₀F₃NO₂ requires 424 |
| 114 | 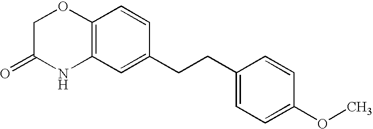 6-[2-(4-Methoxy-phenyl)-ethyl]-4H-benzo[1,4]oxazin-3-one | MS: (ES⁺) 284 m/z(M + 1)⁺C₁₇H₁₇NO₃ requires 284 |
| 115 | 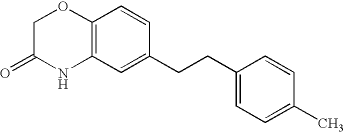 6-(2-p-Tolyl-ethyl)-4H-benzo[1,4]oxazin-3-one | MS: (ES⁺) 268 m/z (M + 1)⁺C₁₇H₁₇NO₂ requires 268 |
| 116 | 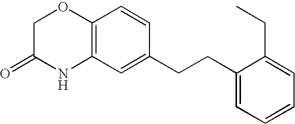 6-[2-(2-Ethyl-phenyl)-ethyl]-4H-benzo[1,4]oxazin-3-one | MS: (ES⁺) 283 m/z(M + 1)⁺C₁₈H₁₉NO₂ requires 283 |
| 117 | 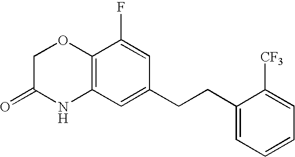 8-Fluoro-6-[2-(2-trifluoromethyl-phenyl)ethyl]-4H-benzo[1,4]oxazin-3-one | MS: (ES⁺) 338 m/z(M + 1)⁺C₁₇H₁₁F₄NO₂ requires 338 |
| 118 | 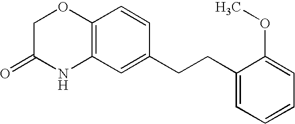 6-[2-(2-Methoxy-phenyl)-ethyl]-4H-benzo[1,4]oxazin-3-one | MS: (ES⁺) 284 m/z(M + 1)⁺C₁₇H₁₇NO₃ requires 284 |
| 119 | 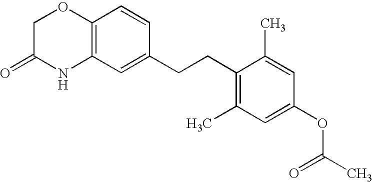 Acetic acid 3,5-dimethyl-4-[2-(3-oxo-3,4-dihydro-2H-benzo[1,4]oxazin-6-yl)-ethyl]-phenyl ester | MS: (ES⁺)340 m/z (M + 1)⁺C₂₀H₂₁NO₄ requires 340 |
| 120 | 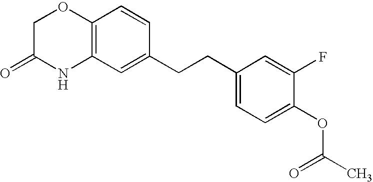 Acetic acid 2-fluoro-4-[2-(3-oxo-3,4-dihydro-2H-benzo[1,4]oxazin-6-yl)-ethyl]-phenyl ester | MS: (ES⁺)330 m/z (M + 1)⁺C₁₈H₁₆FNO₄ requires 330 |
| 121 | 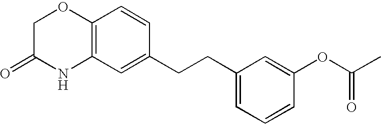 Acetic acid 3-[2-(3-oxo-3,4-dihydro-2H-benzo[1,4]oxazin-6-yl)-ethyl]-phenylester | MS: (ES⁺)312 m/z (M + 1)⁺C₁₈H₁₈NO₄ requires 312 |
| 122 | 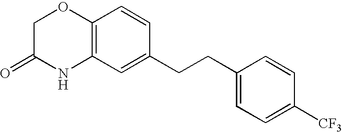 6-[2-(4-Trifluoromethyl-phenyl)-ethyl]-4H-benzo[1,4]oxazin-3-one | MS: (ES⁺) 322 m/z(M + 1)⁺C₁₇H₁₅F₃NO₂ requires 322 |

Compounds from table 3 were prepared according to reference 3.

TABLE 3

| | | Physical Data |
|---|---|---|
| | | ¹H NMR 400 MHz |
| Compound | | (CDCl₃ or DMSO) and/or |
| Number | Structure | MS (m/z) (M + 1)⁺ |
| 123 | 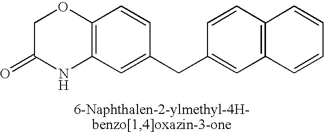 | ¹H NMR (400MHz, DMSO-_{d6}) δ 7.82-7.76(m, 3 H), 7.61 (s, 1 H),7.53-7.42 (m, 3 H), 6.92-6.85(m, 2 H), 6.59 (d, J =1.6 Hz, 1 H), 4.58 (s, 1 H),4.07 (s, 2 H); MS: (ES⁺) 290m/z (M + 1)⁺ C₁₉H₁₆NO₂requires 290 |
| 124 | 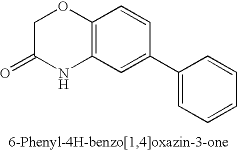 | ¹H NMR (400 MHz,CDCl₃) δ 8.78 (s, 1 H),7.43-7.47 (m, 2 H), 7.33-7.38(m, 2 H), 7.25-7.29(m, 1 H), 7.14 (dd, J = 2, 8Hz, 1 H), 6.96-6.98 (m,2 H), 4.40 (s, 2 H), MS:(ES⁺) 226 m/z (M + 1)⁺C₁₄H₁₂NO₂ requires 226 |
| 125 | 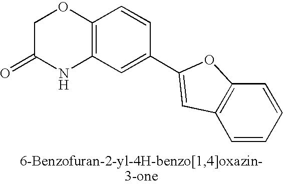 | MS: (ES⁺) 266 m/z (M + 1)⁺C₁₆H₁₂NO₃ requires 266 |
| 126 | 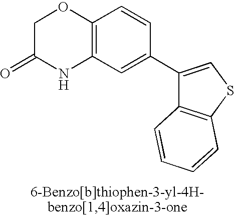 | ¹H NMR (400 MHz,CDCl₃) δ 8.21 (s, 1 H),7.84-7.93 (m, 2 H), 7.39-7.42(m, 2 H), 7.36 (s, 1 H),7.20-7.22 (m, 1 H), 7.09 (d,J = 8.4, 1 H), 7.00-7.01(m, 1 H), MS: (ES⁺) 282m/z (M + 1)⁺ C₁₆H₁₂NO₂Srequires 282 |
| 127 | 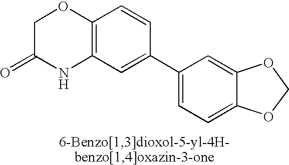 | ¹H NMR (400 MHz,CDCl₃) δ 8.20 (s, 1 H), 7.01(s, 1 H), 6.88 (dd, J = 1.4, 8Hz, 1 H), 6.68-6.77 (m,4 H), 6.61 (d, J = 8 Hz,1 H), 5.75 (s, 2 H), 4.40 (s,2 H), MS: (ES⁺) 270 m/z(M + 1)⁺ C₁₅H₁₂NO₄requires 270 |
| 128 | 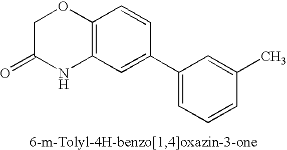 | . ¹H NMR (400 MHz,CDCl₃) δ 8.71 (s, 1 H),7.31-7.33 (m, 3 H), 7.26 (s,1 H), 7.20 (dd, J = 1.4, 8Hz, 1 H), 7.15-7.17 (m,1 H), 7.04 (s, 1 H), 7.02 (d,J = 1.4 Hz, 1 H), 4.66 (s,2 H), 2.42 (s, 3 H), MS:(ES⁺) 240 m/z (M + 1)⁺C₁₅H₁₄NO₂ requires 240 |
| 129 | 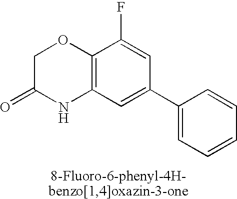 | . ¹H NMR (400 MHz,CDCl₃) δ 7.88 (s, 1 H),7.27-7.31 (m, 2 H), 7.21-7.25(m, 2 H), 7.14-7.18(m, 1 H), 6.84 (dd, J = 2, 11Hz, 1 H), 6.59 (t, J = 1.6Hz, 1 H), 4.52 (s, 2 H), MS:(ES⁺) 244 m/z (M + 1)⁺C₁₄H₁₁FNO₂ requires 244 |
| 130 | 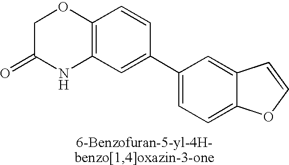 | ¹H NMR (400 MHz,CDCl₃) δ 8.09 (s, 1 H), 7.62(d, J = 1.6, 1 H), 7.57 (d, J =2.4, 1 H), 7.45 (d, J = 8.4Hz, 1 H), 7.34 (dd, J = 2,8.8, 1 H), 7.12-7.16 (m,1 H), 6.96 (d, J = 8.4 Hz,1 H), 6.93 (d, J = 2 Hz,1 H), 6.72 (dd, J = 0.8, 1.6Hz, 1 H), 4.58 (s, 2 H), MS:(ES⁺) 266 m/z (M + 1)⁺C₁₆H₁₂NO₃ requires 266 |
| 131 | 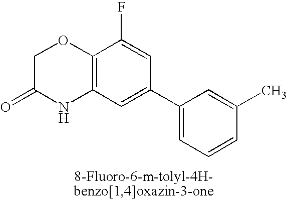 | ¹H NMR (400 MHz,CDCl₃) δ 8.64 (s, 1 H),7.21-7.29 (m, 3 H), 7.12 (d,J = 7.2 Hz, 1 H), 6.99 (dd, J =2, 11.2 Hz, 1 H), 6.76 (t,J = 1.6 Hz, 1 H), 4.67 (s,2 H), 2.36 (s, 3 H), MS:(ES⁺) 258 m/z (M + 1)⁺C₁₅H₁₃FNO₂ requires 258 |
| 132 | 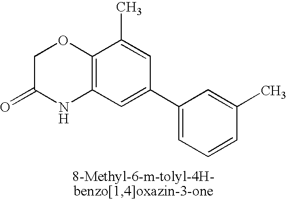 | ¹H NMR (400 MHz,CDCl₃) ) δ 8.42 (s, 1 H),7.21-7.29 (m, 3 H), 7.12(m, 2 H), 6.82 (m, 1 H),4.41 (s, 2 H), 2.36 (s, 3 H),2.28 (s, 3 H), MS: (ES⁺)254 m/z (M + 1)⁺C₁₆H₁₆NO₂ requires 254 |
| 133 | 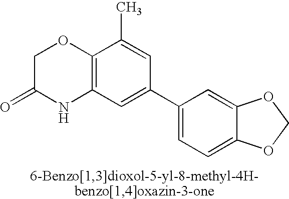 | ¹H NMR (600 MHz,DMSO-_{d6}) δ 10.71 (s, 1 H),7.07-7.08 (m, 1 H), 7.04-7.05(m, 1 H), 6.94-6.98(m, 2 H), 6.88-6.89 (m,1 H), 6.05 (s, 2 H), 4.60 (s,2 H), 2.21 (s, 3 H), MS:(ES⁺) 284 m/z (M + 1)⁺C₁₆H₁₄NO₄ requires 284 |
| 134 | 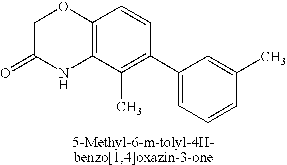 | ¹H NMR (600 MHz,DMSO-_{d6}) δ 10.17 (s, 1 H),7.23 (t, J = 7.2 Hz, 1 H),7.09 (d, J = 7.8 Hz, 1 H),7.00 (s, 1 H), 6.97 (d, J =7.8 Hz, 1 H), 6.81 (d, J =8.2 Hz, 1 H), 6.72 (d, J =8.2 Hz, 1 H), 4.48 (s, 2 H),2.43 (s, 3 H), 2.21 (s, 3 H),MS: (ES⁺) 254 m/z (M + 1)⁺C₁₆H₁₆NO₂ requires 254 |
| 135 | 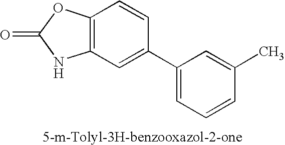 | ¹H NMR (400 MHz,CDCl₃) δ 8.59 (s, 1 H),7.36-7.32 (m, 4 H), 7.28-7.25(m, 2 H), 7.19-7.18(m, 1 H), 2.43 (s, 3 H); MS:(ES⁺) 226 m/z (M + 1)⁺C₁₄H₁₂NO₂ requires 226 |
| 136 | 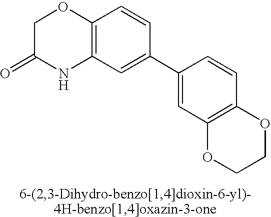 | ¹H NMR (400 MHz,CDCl₃) δ 8.00 (s, 1 H), 7.14(dd, J = 2.0, 8.4 Hz, 1 H),7.05-6.97 (m, 3 H), 6.92 (s,2 H), 4.64 (s, 2 H), 4.30 (s,4 H); MS: (ES⁺) 284 m/z(M + 1)⁺ C₁₆H₁₄NO₄requires 284 |
| 137 | 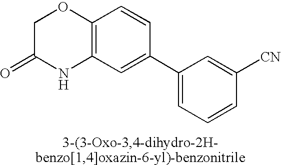 | MS: (ES⁺) 251 m/z (M + 1)⁺C₁₅H₁₁N₂O₂ requires 251 |
| 138 | 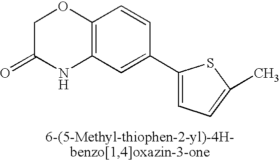 | . ¹H NMR (400 MHz,CDCl₃) δ 8.18 (s, 1 H), 7.16(dd, J = 2.0, 7.6 Hz, 1 H),7.00-6.95 (m, 3 H), 6.71-6.70(m, 1 H), 4.64 (s, 2 H),2.50 (s, 3 H); MS: (ES⁺)246 m/z (M + 1)⁺C₁₃H₁₂NO₂S requires 246 |
| 139 | 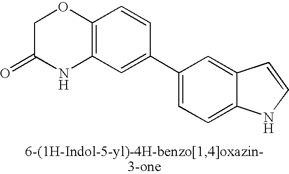 | ¹H NMR (400 MHz,CDCl₃) δ 8.22 (br s, 1 H),7.74 (s, 1 H), 7.73 (br s,1 H), 7.51-7.35 (m, 3 H),7.06-7.03 (m, 2 H), 6.60 (s,1 H), 4.66 (s, 2 H); MS:(ES⁺) 265 m/z (M + 1)⁺C₁₆H₁₃N₂O₂ requires 265 |
| 140 | 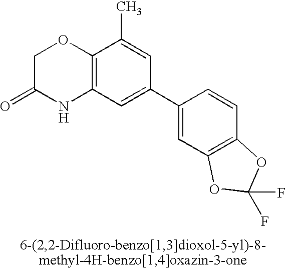 | MS: (ES⁺) 320 m/z (M + 1)⁺C₁₆H₁₂F₂NO₄ requires 320 |
| 141 | 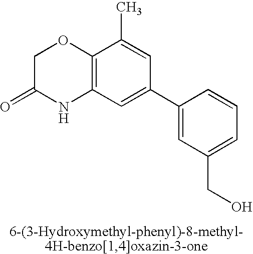 | . ¹H NMR (400 MHz,DMSO-_{d6}) δ 10.71 (s, 1 H),7.51-7.49 (m, 1 H), 7.40-7.38(m, 2 H), 7.26-7.24(m, 1 H), 7.11 (s, 1 H), 6.98(d, J = 2.0 Hz, 1 H), 5.25(t, J = 6.0 Hz, 1 H), 4.62 (s,2 H), 4.55 (d, J = 6.0 Hz,1 H), 2.23 (s, 3 H); MS:(ES⁺) 270 m/z (M + 1)⁺C₁₆H₁₆NO₃ requires 270 |
| 142 | 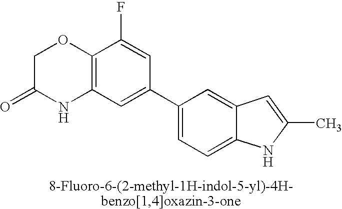 | . ¹H NMR (400 MHz,CD₃OD) δ 7.52 (d, J = 1.2Hz, 1 H), 7.24 (d, J = 8.4Hz, 1 H), 7.15 (dd, J = 2.0,8.4 Hz, 1 H), 7.01 (dd, J =8.4 Hz, 1 H), 6.92 (t, J =1.2 Hz, 1 H), 6.11 (s, 1 H),4.65 (s, 2 H), 2.42 (s, 3 H);MS: (ES⁺) 297 m/z (M + 1)⁺C₁₇H₁₄FN₂O₂ requires 297 |
| 143 | 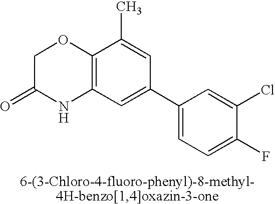 | ¹H NMR (400 MHz,DMSO-_{d6}) δ 10.72 (s, 1 H),7.72 (dd, J = 2.0, 6.8 Hz,1 H), 7.56-7.46 (m, 2 H),7.16-7.15 (m, 1 H), 6.95 (d,J = 2.0 Hz, 1 H), 4.63 (s,2 H), 2.22 (s, 3 H); MS:(ES⁺) 292 m/z (M + 1)⁺C₁₅H₁₂ClFNO₂ requires292 |
| 144 | 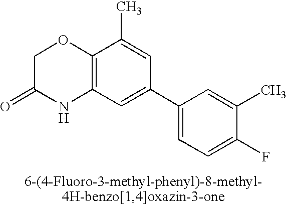 | . ¹H NMR (400 MHz,DMSO-_{d6}) δ 10.71 (s, 1 H),7.45 (dd, J = 2.0, 7.6 Hz,1 H), 7.38-7.32 (m, 1 H),7.19 (t, J = 9.6 Hz, 1 H),7.08 (d, J = 1.6 Hz, 1 H),6.93 (d, J = 2.0 Hz,1 H), 2.28 (d, J = 1.6 Hz,1 H), 2.21 (s, 3 H); MS:(ES⁺) 272 m/z (M + 1)⁺C₁₆H₁₅FNO₂ requires 272 |
| 145 | 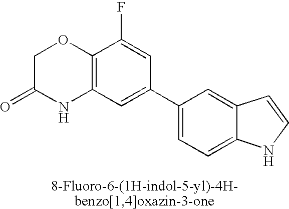 | MS: (ES⁺) 283 m/z (M + 1)⁺C₁₆H₁₂FN₂O₂ requires 283 |
| 146 | 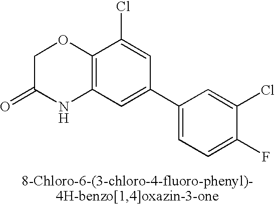 | MS: (ES⁺) 313 m/z (M + 1)⁺C₁₄H₉Cl₂FNO₂ requires313 |
| 147 | 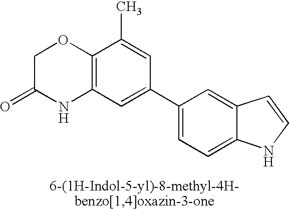 | ¹H NMR (400 MHz,DMSO-_{d6}) δ 9.36 (s, 1 H),8.61 (s, 1 H), 7.74 (s, 1 H),7.48 (d, J = 8.4 Hz, 1 H),7.35 (dd, J = 2.0, 8.4 Hz,1 H), 7.29 (t, J = 2.8 Hz,1 H), 7.14 (s, 1 H), 7.00 (d,J = 2.0 Hz, 1 H), 6.51 (t, J =2.0 Hz, 1 H), 4.58 (s,2 H), 2.70 (s, 3 H); MS:(ES⁺) 279 m/z (M + 1)⁺C₁₇H₁₅N₂O₂ requires 279 |
| 148 | 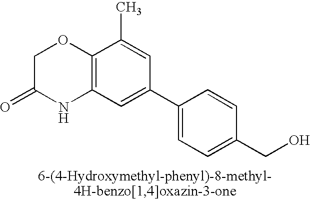 | . ¹H NMR (400 MHz,DMSO-_{d6}) δ 10.71 (s, 1 H),7.49 (d, J = 8.4 Hz, 2 H),7.37 (d, J = 8.4 Hz, 2 H),7.11 (d, J = 1.6 Hz, 1 H),6.98 (d, J = 1.6 Hz, 1 H),5.21 (t, J = 5.6 Hz, 1 H),4.61 (s, 2 H), 4.52 (d, J =5.6 Hz, 1 H), 2.20 (s, 3 H);MS: (ES⁺) 270 m/z (M + 1)⁺C₁₆H₁₆NO₃ requires 270 |
| 149 | 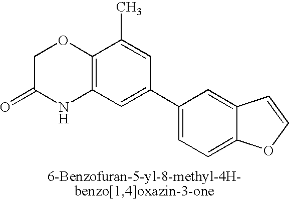 | ¹H NMR (400 MHz,CDCl₃) δ 7.30 (br s, 1 H),7.71 (d, J = 1.6 Hz, 1 H),7.66 (d, J = 2.0 Hz, 1 H),7.55 (d, J = 8.4 Hz, 1 H),7.43 (dd, J = 2.0, 8.4 Hz,1 H), 7.11 (s, 1 H), 6.85 (d,J = 2.0 Hz, 1 H), 6.81 (d, J =1.2 Hz, 1 H), 4.68 (s,2 H), 2.31 (s, 3 H) MS:(ES⁺) 280 m/z (M + 1)⁺C₁₇H₁₄NO₃ requires 280 |
| 150 | 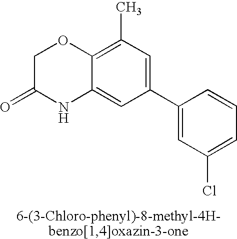 | ¹H NMR (400 MHz,DMSO-_{d6}) δ, 10.70 (s, 1 H),7.59-7.56 (m, 1 H), 7.52-7.44(m, 2 H), 7.41-7.37(m, 1 H), 7.18 (d, J = 1.6Hz, 1 H), 6.98 (d, J = 1.6Hz, 1 H), 4.63 (s, 2 H), 2.22(s, 3 H). MS: (ES⁺) 274m/z (M + 1)⁺ C₁₅H₁₂ClNO₂requires 274 |
| 151 | 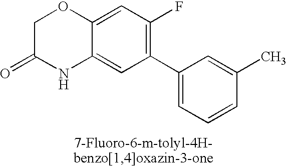 | ¹H NMR (400 MHz,DMSO-_{d6}) δ, 10.70 (s, 1 H),7.38-7.32 (m, 1 H), 7.26-7.18(m, 3 H), 6.99 (d, J =11.2 Hz, 1 H), 6.94 (d, J =7.6 Hz, 1 H), 4.64 (s, 2 H),2.35 (s, 3 H). MS: (ES⁺)258 m/z (M + 1)⁺C₁₅H₁₂FNO₂ requires 258 |
| 152 | 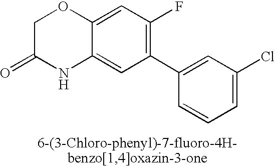 | ¹H NMR (400 MHz,DMSO-_{d6}) δ, 10.70 (s, 1 H),7.51-7.39 (m, 4 H), 7.03 (d,J = 11.2 Hz, 1 H), 6.96 (d, J =8.0 Hz, 1 H), 4.64 (s,2 H). MS: (ES⁺) 278 m/z(M + 1)⁺ C₁₄H₉ClFNO₂requires 278 |
| 153 | 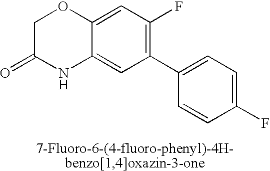 | ¹H NMR (400 MHz,DMSO-_{d6}) δ, 10.70 (s, 1 H),7.52-7.46 (m, 2 H), 7.34-7.26(m, 2 H), 7.02 (d, J =11.2 Hz, 1 H), 6.93 (d, J =8.0 Hz, 1 H), 4.64 (s, 2 H).MS: (ES⁺) 262 m/z(M + 1)⁺ C₁₄H₉F₂NO₂requires 262 |
| 154 | 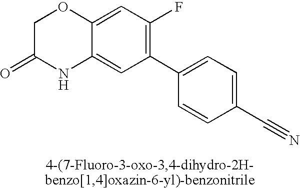 | ¹H NMR (400 MHz,DMSO-_{d6}) δ, 10.70 (s, 1 H),7.96-7.92 (m, 2 H), 7.67-7.64(m, 2 H), 7.07 (d, J =11.2 Hz, 1 H), 6.99 (d, J =8.0 Hz, 1 H), 4.64 (s, 2 H).MS: (ES⁺) 269 m/z(M + 1)⁺ C₁₅H₉FN₂O₂requires 269 |
| 155 | 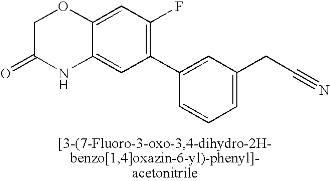 | . ¹H NMR (400 MHz,DMSO-_{d6}) δ, 10.70 (s, 1 H),7.52-7.35 (m, 3 H), 7.10(dd, J = 12.0 Hz, 9.2 Hz,1 H), 7.03 (d, J = 11.2 Hz,1 H), 6.96 (d, J = 7.6 Hz,1 H), 4.64 (s, 2 H), 4.12 (s,2 H). MS: (ES⁺) 283 m/z(M + 1)⁺ C₁₆H₁₁FN₂O₂requires 283 |
| 156 | 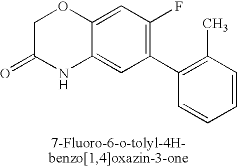 | . ¹H NMR (400 MHz,DMSO-_{d6}) δ, 10.70 (s, 1 H),7.33-7.23 (m, 3 H), 7.15 (d,J = 7.2 Hz, 1 H), 6.90 (d, J =10.4 Hz, 1 H), 6.73 (d, J =7.2 Hz, 1 H), 4.65 (s,2 H), 2.13 (s, 3 H). MS:(ES⁺) 258 m/z (M + 1)⁺C₁₂H₁₂FNO₂ requires 258 |
| 157 | 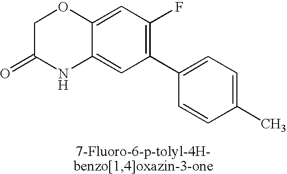 | ¹H NMR (400 MHz,DMSO-_{d6}) δ, 10.20 (s, 1 H),7.36-7.26 (m, 4 H), 6.98 (d,J = 11.2 Hz, 1 H), 6.93 (d, J =8.0 Hz, 1 H), 4.62 (s,2 H), 2.34 (s, 3 H). MS:(ES⁺) 258 m/z (M + 1)⁺C₁₅H₁₂FNO₂ requires 258 |
| 158 | 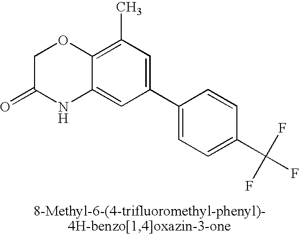 | ¹H NMR (400 MHz,DMSO-_{d6}) δ 9.21 (s, 1 H),8.32 (s, 4 H), 7.77 (d, J =1.6 Hz, 1 H), 7.61 (d, J =2.0 Hz, 1 H), 5.19 (s, 2 H),5.86 (s, 3 H). MS: (ES⁺)308 m/z (M + 1)⁺C₁₆H₁₃F₃NO₂ requires 308 |
| 159 | 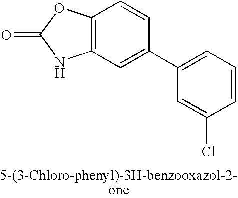 | ¹H NMR (400 MHz,DMSO-_{d6}) δ 11.73 (s, 1 H),7.62 (t, J = 2.0 Hz, 1 H),7.52-7.54 (m, 1 H), 7.40 (t,J = 8.0 Hz, 1 H), 7.34-7.35(m, 1 H), 7.29-7.31 (m,2 H), 7.27 (d, J = 1.6 Hz,1 H). MS: (ES⁺) 246 m/z(M + 1)⁺ C₁₃H₉ClNO₂requires 246 |
| 160 | 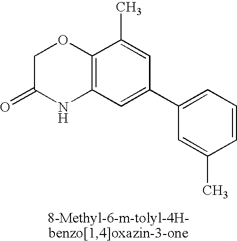 | ¹H NMR (400 MHz,DMSO-_{d6}) δ 10.62 (s, 1 H),7.26 (s, 1 H), 7.22-7.24 (m,2 H), 7.05 (t, J = 4.0 Hz,1 H), 7.01 (d, J = 1.6 Hz,1 H), 7.89 (d, J = 2.4 Hz,1 H), 4.53 (s, 2 H), 2.27 (s,3 H), 2.14 (s, 1 H). MS:(ES⁺) 254 m/z (M + 1)⁺C₁₆H₁₆NO₂ requires 254 |
| 161 | 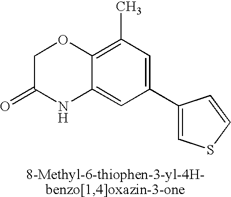 | ¹H NMR (400 MHz,DMSO-_{d6}) δ 10.60 (s, 1 H),7.56-7.52 (m, 2 H), 7.29(dd, J = 3.6 Hz, 4.8 Hz, 1 H),7.08 (d, J = 1.6 Hz, 1 H),6.91 (d, J = 1.6 Hz, 1 H),4.52 (s, 2 H), 2.12 (s, 3 H).MS: (ES⁺) 246 m/z (M + 1)⁺C₁₃H₁₂NO₂S requires 246 |
| 162 | 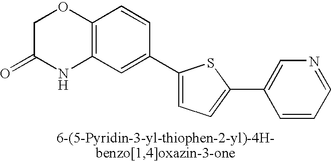 | ¹H NMR (400 MHz,DMSO-_{d6}) δ 10.71 (s, 1 H),8.85 (d, J = 2.0 Hz, 1 H),8.43 (dd, J = 3.2 Hz, 4.8 Hz,1 H), 8.01-7.98 (m, 1 H),7.57 9(d, J = 4.0 Hz, 1 H),7.40-7.37 (m, 1 H), 7.33 (d,J = 3.6 Hz, 1 H), 7.21 (dd,J = 6.0 Hz, 8.0 Hz, 1 H), 7.10(d, J = 2.0 Hz, 1 H) 6.93 (d,J = 8.4 Hz, 1 H), 4.54 (s,2 H). MS: (ES⁺) 309 m/z(M + 1)⁺ C₁₇H₁₃N₂O₂Srequires 309 |
| 163 | 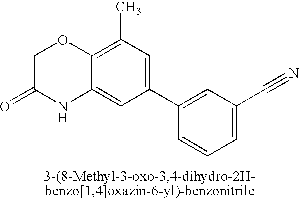 | ¹H NMR (400 MHz,DMSO-_{d6}) δ 10.65 (s, 1 H),7.91 (s, 1 H), 7.79 (d, J =7.6 Hz, 1 H), 7.70 (d, J =7.6 Hz, 1 H), 7.58-7.54 (m,1 H), 7.14 (s, 1 H), 6.93 (s,1 H), 4.55 (s, 2 H), 2.15 (s,3 H). MS: (ES⁺) 265 m/z(M + 1)⁺ C₁₆H₁₃N₂O₂requires 265 |
| 164 | 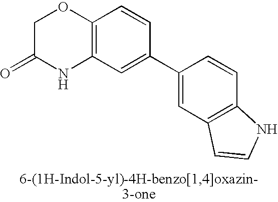 | ¹H NMR (400 MHz,DMSO-_{d6}) δ 8.14 (s, 1 H),7.71 (s, 1 H), 7.66 (s, 1 H),7.37 (t, J = 8.0 Hz, 1 H),7.28-7.31 (m, 1 H), 6.96-6.99(m, 2 H), 6.54 (s, 1 H),4.59 (s, 1 H), . MS: (ES⁺)265 m/z (M + 1)⁺C₁₆H₁₃N₂O₂ requires 265 |
| 165 | 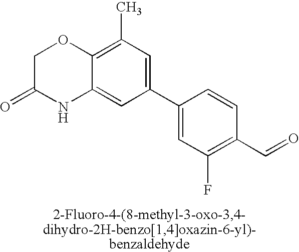 | . ¹H NMR (400 MHz,DMSO-_{d6}) δ 9.24 (s, 1 H),4.01 (s, 3 H). MS: (ES⁺)286 m/z (M + 1)⁺C₁₆H₁₃FNO₃ requires 286 |
| 166 | 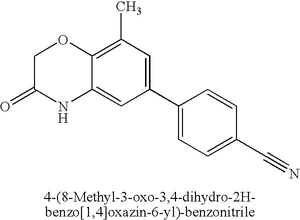 | ¹H NMR (400 MHz,DMSO-_{d6}) δ 10.70 (s, 1 H),7.81 (d, J = 8.0 Hz, 2 H),7.66, (d, J = 8.0 Hz, 2 H),7.15 (s, 1 H), 6.96 (s, 1 H),4.57 (s, 2 H), 2.15 (s, 3 H).MS: (ES⁺) 265 m/z (M + 1)⁺C₁₆H₁₃N₂O₂ requires 265 |
| 167 | 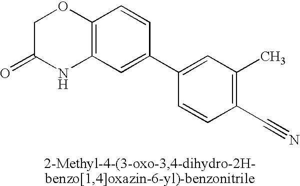 | ¹H NMR (400 MHz,DMSO-_{d6}) δ 10.71 (s, 1 H),7.74 (d, J = 8.4 Hz, 1 H),7.58 (s, 1 H), 7.46, (dd, J =6.4 Hz, 8.0 Hz, 1 H), 7.22(dd, J = 6.4 Hz, 8.4 Hz, 1 H),7.11 (d, J = 2.0 Hz, 1 H),6.98 (d, J = 8.4 Hz, 1 H),4.55 (s, 2 H), 2.46 (s, 3 H).MS: (ES⁺) 265 m/z (M + 1)⁺C₁₆H₁₃N₂O₂ requires 265 |
| 168 | 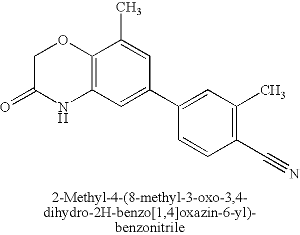 | ¹H NMR (400 MHz,DMSO-_{d6}) δ 10.65 (s, 1 H),7.73 (d, J = 8.0 Hz, 1 H),7.57 (s, 1 H), 7.45 (dd, J =6.4 Hz, 8.0 Hz, 1 H), 7.13 (d,J = 1.6 Hz, 1 H), 6.95 (d, J =2.0 Hz, 1 H), 4.56 (s, 2 H),2.45 (s, 3 H), 2.15 (s, 3 H).MS: (ES⁺) 279 m/z (M + 1)⁺C₁₇H₁₅N₂O₂ requires 279 |
| 169 | 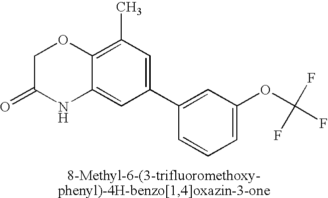 | . ¹H NMR (400 MHz,DMSO-_{d6}) δ 10.62 (s, 1 H),7.50 (d, J = 0.8 Hz, 1 H),7.49-7.48 (m, 1 H), 7.4 (d,J = 0.8 Hz, 1 H), 7.25-7.23(m, 1 H), 7.09 (d, J = 1.6 Hz,1 H), 6.93 (d, J = 2.0 Hz,1 H), 4.55 (s, 2 H), 2.15 (s,3 H). MS: (ES⁺) 324 m/z(M + 1)⁺ C₁₆H₁₃F₃NO₃requires 324 |
| 170 | 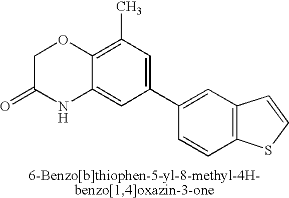 | . ¹H NMR (400 MHz,DMSO-_{d6}) δ 7.82-7.87 (m,2 H), 7.41-7.45 (m, 2 H),7.31 (d, J = 5.6 Hz, 1 H),7.08 (s, 1 H), 6.83 (d, J =1.6 Hz, 1 H), 4.62 (s, 2 H),2.26 (s, 3 H). MS: (ES⁺)296 m/z (M + 1)⁺C₁₇H₁₄NO₂S requires 296 |
| 171 | 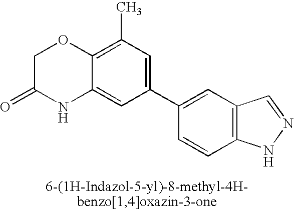 | ¹H NMR (400 MHz,DMSO-_{d6}) δ 10.49 (s, 1 H),8.03 (s, 1 H), 7.79 (s, 1 H),9.44 (q, J = 8.4 Hz, 2 H),7.05 (d, J = 1.6 Hz, 1 H),6.92 (d, J = 2.0 Hz, 1 H),4.54 (s, 2 H), 2.15 (s, 3 H).MS: (ES⁺) 280 m/z (M + 1)⁺C₁₆H₁₃N₃O₂ requires 280 |
| 172 | 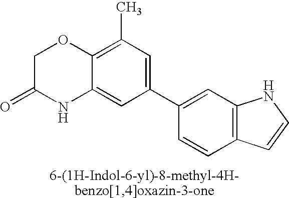 | ¹H NMR (400 MHz,DMSO-_{d6})δ 11.33 (s, 1 H), 11.28 (s,1 H), 8.30 (d, J = 8.0 Hz,1 H), 8.26 (s, 1 H), 7.96-7.95(m, 1 H), 7.93 (d, J =1.6 Hz, 1 H), 7.77 (q, J =1.2 Hz, 2 H), 7.17-7.16 (m,1 H), 5.30 (s, 2 H), 3.01 (s,3 H). MS: (ES⁺) 279 m/z(M + 1)⁺ C₁₇H₁₅N₂O₂requires 279 |
| 173 | 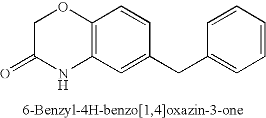 | ¹H NMR (400 MHz,CDCl₃) δ 7.38-7.24 (m,4 H), 7.02-6.86 (m, 4 H),5.16 (s, 2 H), 4.74 (s, 2 H),MS: (ES⁺) 239 m/z (M + 1)⁺C₁₅H₁₃NO₂ requires 240 |
| 174 | 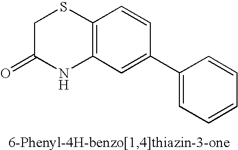 | ¹H NMR (400 MHz,CDCl₃) δ 8.39 (s, 1 H),7.56-7.52 (m, 2 H), 7.48-7.42(m, 2 H), 7.40-7.35(m, 2 H), 7.27-7.23 (m,1 H), 7.06 (d, J = 2.0 Hz,1 H). MS: (ES⁺) 242 m/z(M + 1)⁺ C₁₄H₁₂NOSrequires 242 |
| 175 | 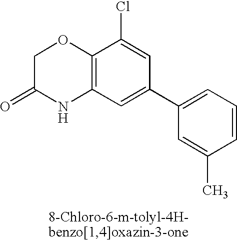 | ¹H NMR (400 MHz,DMSO-_{d6}) δ 10.94 (s, 1 H),7.38 (s, 1 H), 7.39-7.32 (m,3 H), 7.22-7.16 (m, 1 H),7.18 (d, J = 2.0 Hz, 1 H),4.74 (s, 2 H), 2.36 (s, 3 H),4.01 (s, 3 H). MS: (ES⁺)275 m/z (M + 1)⁺C₁₅H₁₂ClNO₂ requires 275 |
| 176 | 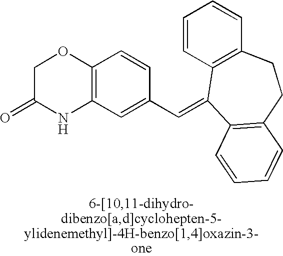 | ¹H NMR (400 MHz,DMSO-_{d6}) δ 7.28 (m, 2 H),7.11 (m, 2 H), 7.09 (m,2 H), 6.93 (m, 3 H), 6.62(dd, J = 8.4, 2.0 Hz, 1 H),6.55 (dt, J = 8.4, 2.4 Hz,1 H), 6.51 (d, J = 2.0 Hz,1 H), 6.10 (s, 1 H), 4.39 (s,2 H), 3.38 (m, 1 H,), 3.29(m, 1 H), 2.88 (m, 1 H),2.72 (m, 1 H); MS: (ES⁺)354 m/z (M + 1)⁺C₂₄H₁₉NO₂ requires 354 |
| 177 | 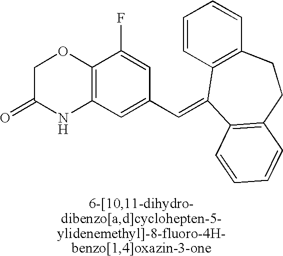 | ¹H NMR (400 MHz,DMSO-_{d6}) δ 7.41 (m, 2 H),7.27 (m, 2 H), 7.21 (m,3 H), 7.07 (m, 2 H), 7.00(m, 1 H), 6.59 (broad s,1 H), 6.50 (m, 1 H), 6.01(broad s, 1 H), 6.10 (s, 1 H),4.62 (s, 2 H), 3.45 (m, 1 H,),3.30 (m, 1 H), 3.02 (m,1 H), 2.89 (m, 1 H); MS:(ES⁺) 372 m/z (M + 1)⁺C₂₄H₁₈FNO₂ requires 372 |
| 178 | 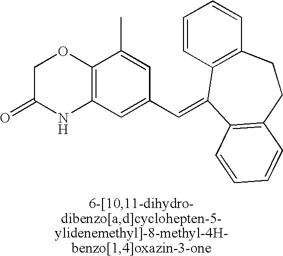 | ¹H NMR (400 MHz,DMSO-_{d6}) δ 7.56 (m, 1 H),7.30 (m, 4 H), 7.18 (m,4 H), 6.75 (t, J = 3.0 Hz,1 H), 6.17 (s, 1 H), 4.68 (s,2 H), 3.63 (m, 1 H,), 3.48(m, 1 H), 3.12 (m, 1 H),2.99 (m, 1 H), 2.23 (s, 3 H);MS: (ES⁺) 368 m/z (M + 1)⁺C₂₅H₂₁NO₂ requires 368 |
| 179 | 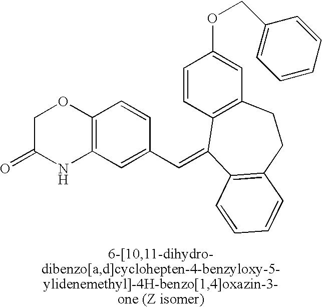 | ¹H NMR (400 MHz,CDCl₃) δ 7.45 (broad s, 1),7.31 (m, 4 H), 7.11 (m,2 H), 7.02 (m, 1 H), 6.90(m, 2 H), 6.86 (d, J = 8.4Hz, 1 H), 6.84 (d, J = 2.4Hz, 1 H), 6.61 (dd, J = 8.4,2.4 Hz, 1 H), 6.60 (dd, J =8.4, 2.4 Hz, 1 H), 6.57 (s,1 H), 6.30 (d, J = 1.6 Hz,1 H), 4.98 (s, 2 H), 4.50 (s,2 H), 3.35 (m, 2 H,), 2.90(m, 2 H); MS: (ES⁺) 459m/z (M + 1)⁺ C₃₁H₂₄NO₃requires 459 |
| 180 | 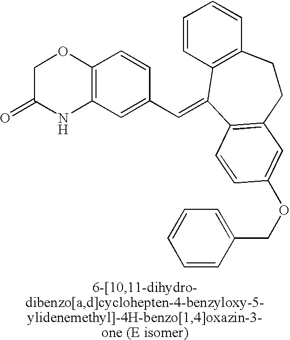 | MS: (ES⁺) 459 m/z (M + 1)⁺C₃₁H₂₄NO₃ requires 459 |
| 181 | 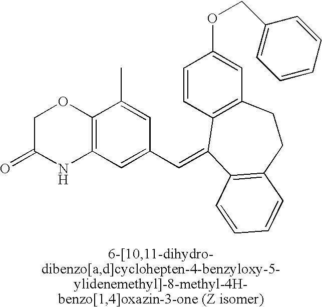 | MS: (ES⁺) 459 m/z (M + 1)⁺C₃₁H₂₄NO₃ requires 459 |
| 182 | 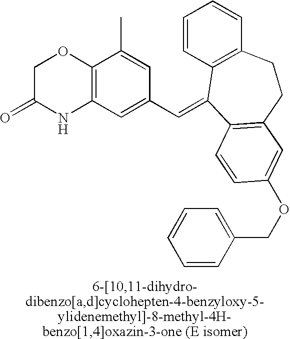 | MS: (ES⁺) 459 m/z (M + 1)⁺C₃₁H₂₄NO₃ requires 459 |
| 183 | 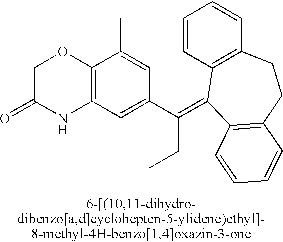 | ¹H NMR (DMSO-d6,400 MHz): 10.47 (s, 1 H),7.21 (m, 1 H), 7.18 (m,2 H), 6.95 (d, J = 8.4 Hz,1 H), 6.79 (td, J = 8.4, 1.6Hz, 1 H), 6.67 (td, J = 8.4,1.6 Hz, 1 H), 6.60 (d, J =1.6 Hz, 1 H), 6.52 (d, J =1.6 Hz, 1 H), 4.51 (s, 2 H),3.42 (m, 2 H), 2.81 (m,2 H), 2.55 (m, 1 H), 2.28(m, 1 H), 2.04 (s, 3 H), 0.67(t, J = 7.1 Hz, 3 H). MS(ES⁺) 395, m/z (M + 1) 396,C₂₇H₂₅NO₂ requires 395 |
| 184 | 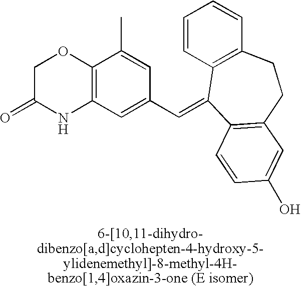 | ¹H NMR (CDCl₃,400 MHz): 7.33 (d, J = 8.4Hz, 1 H), 7.20 (m, 2 H),7.06 (td, J = 7.6, 1.6 Hz,1 H), 6.99 (d, J = 6.4 Hz,1 H), 6.89 (d, J = 8.4 Hz,1 H), 6.66 (m, 2 H), 6.58(broad s, 1 H), 6.02 (d, J =1.6 Hz, 1 H), 4.56 (s, 2 H),3.47 (m, 1 H), 3.59 (m,1 H), 3.00 (m, 1 H), 2.63(m, 1 H), 2.11 (s, 3 H). MS(ES⁺) 385, m/z (M + 1) 386,C₂₅H₂₃NO₃ requires 385 |
| 185 | 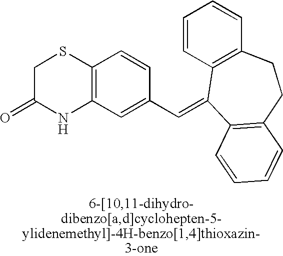 | MS (ES⁺) 369, m/z (M + 1)370, C₂₄H₁₉NOS requires369 |
| 186 | 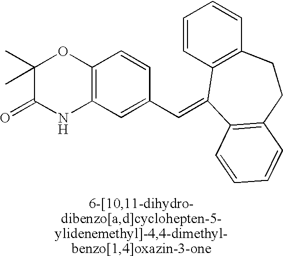 | MS (ES⁺) 381, m/z (M + 1)382, C₂₆H₂₃NO₂ requires381 |
| 187 | 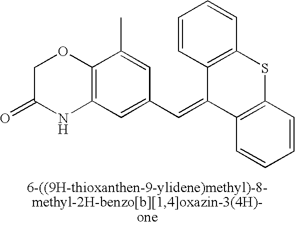 | MS (ES⁺) 371, m/z (M + 1)372, C₂₃H₁₇NO₂S requires371 |
| 188 | 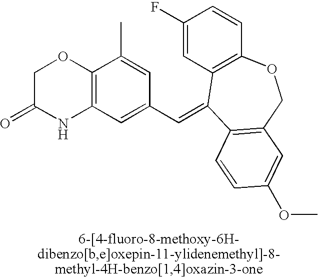 | ¹H NMR (CDCl₃,400 MHz): 7.56 (broad s,1 H), 7.15 (dd, J = 9.6, 3.2Hz, 1 H), 7.01 (d, J = 8.4Hz, 1 H), 6.98 (d, J = 2.4Hz, 1 H), 6.86 (m, 1 H),6.76 (m, 3 H), 6.62 (s, 1 H),6.17 (s, 1 H), 5.21 (broad s,2 H), 4.59 (s, 2 H), 3.85 (s,3 H), 2.13 (s, 3 H). MS(ES⁺) 417, m/z (M + 1) 418,C₂₅H₂₀FNO₄ requires 417 |
| 189 | 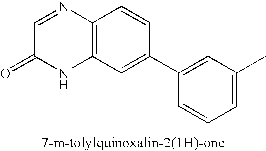 | ¹H NMR (MeOD,400 MHz): 8.20 (s, 1 H),7.89 (d, J = 8.4 Hz, 1 H),7.67 (s, 1 H), 7.64 (dd, J =8.4, 2.0 Hz, 1 H), 7.53 (m,2 H), 7.49 (d, J = 7.6 Hz,1 H), 7.38 (t, J = 7.6 Hz,1 H), 7.25 (d, J = 7.6 Hz,1 H), 2.44 (s, 3 H). MS(ES⁺) 236, m/z (M + 1) 237,C₁₅H₁₂N₂O requires 236 |

Compounds from table 4 were prepared according to reference 6.

TABLE 4

| | | Physical Data |
|---|---|---|
| | | ¹H NMR 400 MHz |
| | | (CDCl₃ or DMSO) |
| Compound | | and/or MS (m/z) |
| Number | Structure | (M + 1)⁺ |
| 190 | 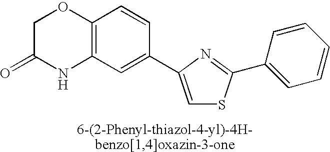 | ¹H NMR (400MHz, DMSO-_{d6}) δ 10.73 (s,1 H), 7.92-7.94 (m, 3 H), 7.56(d, J = 1.6 Hz, 1 H), 7.43-7.52(m, 4 H), 6.97 (d, J = 8Hz, 1 H), 4.56 (s, 2 H). MS:(ES⁺) 309 m/z (M + 1)⁺C₁₇H₁₃N₂O₂S requires 309 |
| 191 | 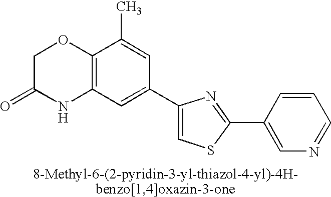 | ¹H NMR (400 MHz,DMSO-_{d6}) δ 10.67 (s, 1 H),8.62-8.63 (m, 1 H), 8.28-8.30(m, 1 H), 7.38-7.54 (m,3 H), 7.19 (t, J = 2 Hz, 1 H),4.58 (s, 2 H), 2.16 (s, 3 H)MS: (ES⁺) 324 m/z (M + 1)⁺C₁₇H₁₄N₃O₂S requires 324 |
| 192 | 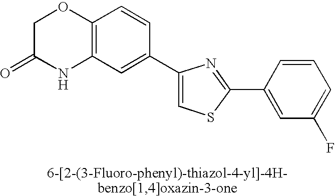 | ¹H NMR (400 MHz,DMSO-_{d6}) δ 10.71 (s, 1 H),7.98 (s, 1 H), 7.71-7.77 (m,2 H), 7.49-7.56 (m, 3 H),7.27-7.32 (m, 1 H), 6.96 (d, J =8.4 Hz, 1 H), 4.55 (s, 2 H).MS: (ES⁺) 327 m/z (M + 1)⁺C₁₇H₁₂FN₂O₂S requires 327 |
| 193 | 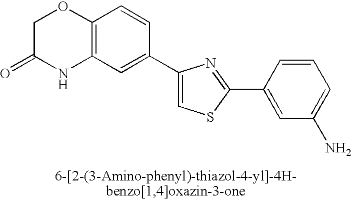 | ¹H NMR (400 MHz,DMSO-_{d6}) δ 10.77 (s, 1 H),7.93 (d, J = 4 Hz, 1 H), 7.47-7.56(m, 4 H), 7.35 (t, J = 8Hz, 1 H), 7.05 (d, J = 8 Hz,1 H), 6.97 (d, J = 8 Hz, 1 H),4.54 (s, 2 H). MS: (ES⁺) 324m/z (M + 1)⁺ C₁₇H₁₄N₃O₂Srequires 324 |
| 194 | 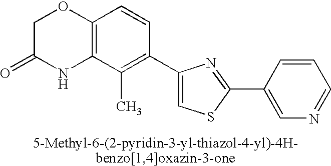 | ¹H NMR (400 MHz,DMSO-_{d6}) δ 10.27 (s, 1 H),9.09 (d, J = 4 Hz, 1 H), 9.08(dd, J = 4.0, 0.8 Hz, 1 H),8.24-8.27 (m, 1 H), 7.75 (s,1 H), 7.47 (dd, J = 8.4 Hz,1 H), 7.11 (d, J = 4 Hz, 1 H),6.85 (d, J = 8 Hz, 1 H), 4.49(s, 2 H), 2.42 (s, 3 H). MS:(ES⁺) 324 m/z (M + 1)⁺C₁₇H₁₄N₃O₂S requires 324 |
| 195 | 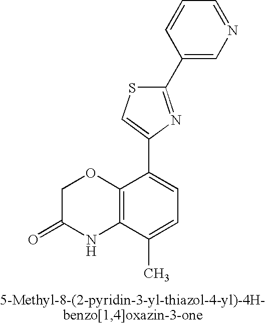 | ¹H NMR (400 MHz,DMSO-_{d6}) δ 10.25 (s, 1 H),9.13 (br s, 1 H), 8.61 (dd, J =4.0, 0.8 Hz, 1 H), 8.29-8.32(m, 1 H), 8.11 (s, 1 H), 7.47-.750(m, 1 H), 6.89 (d, J =4 Hz, 1 H), 6.85 (d, J = 8 Hz,1 H), 4.49 (s, 2 H), 2.42 (s,3 H). MS: (ES⁺) 324 m/z(M + 1)⁺ C₁₇H₁₄N₃O₂Srequires 324 |
| 196 | 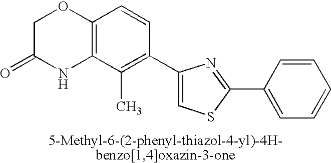 | ¹H NMR (400 MHz,DMSO-_{d6}) δ 10.25 (s, 1 H),9.13 (br s, 1 H), 8.59 (s, 1 H),7.88-7.91 (m, 1 H), 7.65 (s,1 H), 7.43-7.47 (m, 1 H), 7.09(d, J = 4 Hz, 1 H), 6.84 (d, J =8 Hz, 1 H), 4.48 (s, 2 H),2.41 (s, 3 H). MS: (ES⁺) 323m/z (M + 1)⁺ C₁₈H₁₅N₂O₂Srequires 323 |
| 197 | 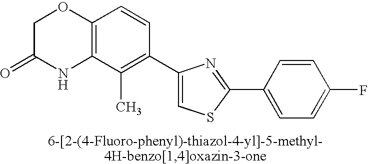 | ¹H NMR (400 MHz,DMSO-_{d6}) δ 10.25 (s, 1 H),7.95 (dd, J = 8.8, 5 Hz, 1 H),7.64 (s, 1 H), 7.28 (t, J = 8.8Hz, 2 H), 7.08 (d, J = 8.4 Hz,1 H), 6.84 (d, J = 8 Hz, 1 H),4.48 (s, 2 H), 2.42 (s, 3 H).MS: (ES⁺) 341 m/z (M + 1)⁺C₁₈H_{14F}N₂O₂S requires 341 |
| 198 | 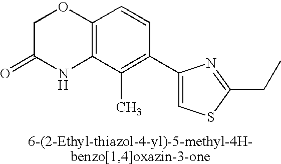 | ¹H NMR (400 MHz,DMSO-_{d6}) δ 10.21 (s, 1 H),7.39 (s, 1 H), 7.00 (d, J =8.4 Hz, 1 H), 6.79 (d, J =8.4 Hz, 1 H), 4.46 (s, 2 H),2.94 (q, J = 15.2, 7.6 Hz,2 H), 2.17 (s, 3 H), 1.24 (t, J =7.6 Hz, 3 H), MS: (ES⁺)275 m/z (M + 1)⁺C₁₄H₁₅N₂O₂S requires 275 |
| 199 | 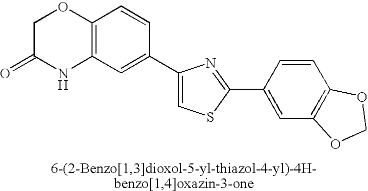 | ¹H NMR (400 MHz,DMSO-_{d6}) δ 10.71 (s, 1 H),7.84 (br s, 1 H), 7.54 (br s,1 H), 7.45-7.48 (m, 3 H),6.94-7.01 (m, 2 H), 4.55 (s,2 H). MS: (ES⁺) 353 m/z(M + 1)⁺ C₁₈H₁₃N₂O₄Srequires 353 |
| 200 | 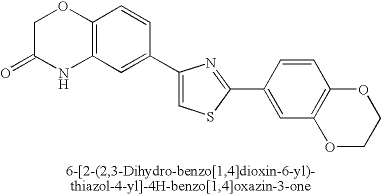 | ¹H NMR (400 MHz,DMSO-_{d6}) δ 10.69 (s, 1 H),7.83 (s, 1 H), 7.54 (s, 1 H),7.46 (d, J = 8 Hz, 1 H), 7.36-7.38(m, 2 H), 6.92-6.95 (m,2 H), 4.54 (s, 2 H), 4.24 (s,4 H). MS: (ES⁺) 367 m/z(M + 1)⁺ C₁₉H₁₅N₂O₄Srequires 367 |
| 201 | 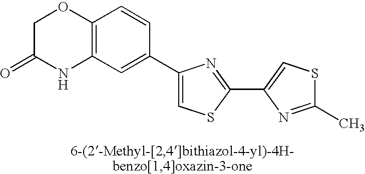 | ¹H NMR (400 MHz,DMSO-_{d6}) δ 10.74 (s, 1 H),7.99 (s, 1 H), 7.89 (s, 1 H),7.46-7.49 (m, 2 H), 6.95 (d, J =8 Hz, 1 H), 4.54 (s, 2 H),2.59 (s, 3 H). MS: (ES⁺) 330m/z (M + 1)⁺ C₁₅H₁₂N₃O₂S₂requires 330 |
| 202 | 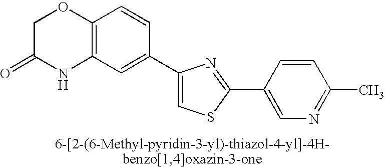 | ¹H NMR (400 MHz,DMSO-_{d6}) δ 10.73 (s, 1 H),8.99 (s, 1 H), 8.20 (d, J = 8Hz, 1 H), 7.98 (s, 1 H), 7.75(s, 1 H), 7.54 (s, 1 H), 7.50(dd, J = 4, 8 Hz, 1 H), 7.41(d, J = 8 Hz, 1 H), 6.96 (d, J =8 Hz, 1 H), 4.55 (s, 2 H),2.43 (s, 1 H). MS: (ES⁺) 324m/z (M + 1)⁺ C₁₇H₁₄N₃O₂Srequires 324 |
| 203 | 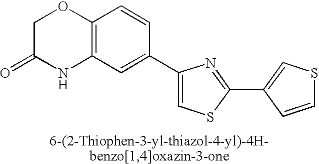 | ¹H NMR (400 MHz,DMSO-_{d6}) δ 10.73 (s, 1 H),8.09 (dd, J = 2.8, 1.2 Hz,1 H), 7.83 (s, 1 H), 7.66 (dd,J = 3.2, 5.2 Hz, 1 H), 7.53(dd, J = 3.2, 5.2 Hz, 1 H),7.45-7.50 (m, 3 H), 6.94 (d, J =8 Hz, 1 H), 4.54 (s, 2 H).MS: (ES⁺) 315 m/z (M + 1)⁺C₁₅H₁₁N₂O₂S₂ requires 315 |
| 204 | 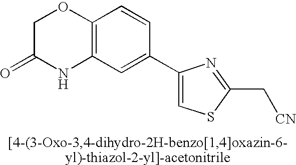 | ¹H NMR (400 MHz,DMSO-_{d6}) δ 10.73 (s, 1 H),7.88 (s, 1 H), 7.40-7.46 (m,3 H), 6.93 (d, J = 8 Hz, 1 H),4.51 (s, 2 H), 3.22 (s, 2 H).MS: (ES⁺) 272 m/z (M + 1)⁺C₁₃H₁₀N₃O₂S requires 272 |
| 205 | 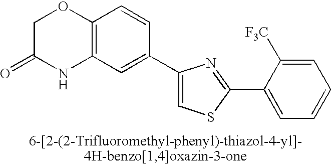 | ¹H NMR (400 MHz,DMSO-_{d6}) δ 10.75 (s, 1 H),8.07 (s, 1 H), 7.88 (d, J =4.0, 0.8 Hz, 1 H), 7.67-7.75(m, 3 H), 7.46-7.49 (m, 2 H),6.96 (d, J = 8 Hz, 1 H), 4.53(s, 2 H). MS: (ES⁺) 377 m/z(M + 1)⁺ C₁₈H₁₂F₃N₂O₂Srequires 377 |
| 206 | 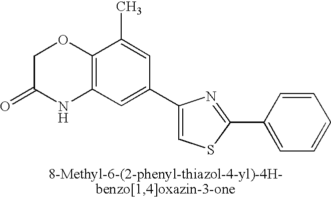 | ¹H NMR (400 MHz,DMSO-_{d6}) δ 10.66 (s, 1 H),7.88-7.93 (m, 3 H), 7.39-7.49(m, 5 H), 4.55 (s, 2 H),2.42 (s, 1 H). MS: (ES⁺) 323m/z (M + 1)⁺ C₁₈H₁₅N₂O₂Srequires 323 |
| 207 | 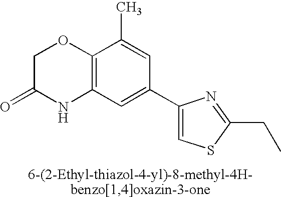 | ¹H NMR (400 MHz,DMSO-_{d6}) δ 10.61 (s, 1 H),7.65 (s, 1 H), 7.28-7.29 (m,H), 4.53 (s, 2 H), 2.94 (q, J =8 Hz, 2 H), 2.42 (s, 1 H), 1.25(t, J = 8 Hz, 3 H). MS: (ES⁺)275 m/z (M + 1)⁺C₁₄H₁₅N₂O₂S requires 275 |
| 208 | 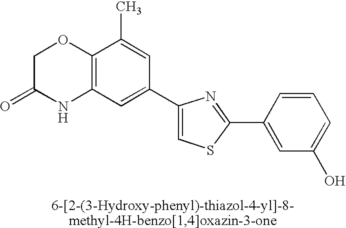 | ¹H NMR (400 MHz,DMSO-_{d6}) δ 10.67 (s, 1 H),9.73 (s, 1 H), 7.85 (s, 1 H),7.31-7.39 (m, 1 H), 7.24 (t, J =8 Hz, 1 H), 6.80-6.83 (m,1 H), 4.55 (s, 2 H), 2.41 (s,3 H). MS: (ES⁺) 339 m/z(M + 1)⁺ C₁₈H₁₅N₂O₃Srequires 339 |
| 209 | 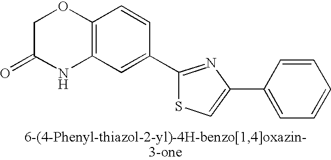 | ¹H NMR (400 MHz,DMSO-_{d6}) δ 10.79 (s, 1 H),8.06 (s, 1 H), 7.95-7.97 (m,2 H), 7.56 (d, J = 2 Hz, 1 H),7.50 (d, J = 8.4, 2 Hz, 1 H),7.42 (t, J = 8.4 Hz, 1 H),7.01 (d, J = 8.4 Hz, 1 H),4.61 (s, 2 H). MS: (ES⁺) 309m/z (M + 1)⁺ C₁₇H₁₃N₂O₂Srequires 309 |
| 210 | 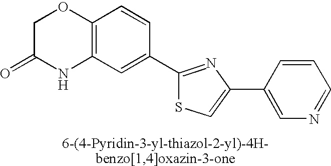 | ¹H NMR (400 MHz,DMSO-_{d6}) δ 10.79 (s, 1 H),9.15 (s, 1 H), 8.50 (d, J = 4.0Hz, 1 H), 8.28 (dt, J = 1.2,7.6 Hz, 1 H), 8.22 (s, 1 H),7.54 (d, J = 2 Hz, 1 H), 7.51(dd, J = 1.2, 7.6 Hz, 1 H),7.41-7.45 (m, 1 H), 7.00 (d, J=8.4 Hz, 1 H), 4.60 (s, 2 H).MS: (ES⁺) 310 m/z (M + 1)⁺C₁₆H₁₂N₃O₂S requires 310 |
| 211 | 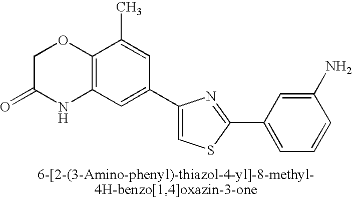 | ¹H NMR (400 MHz,DMSO-_{d6}) δ 10.67 (s, 1 H),7.79 (s, 1 H), 7.36 (dd, J =2.0, 16 Hz, 2 H), 7.13 (br s,1 H), 7.09-7.02 (m, 3 H), 6.59(d, J = 8.4 Hz, 1 H), 4.55 (s,2 H), 2.15 (s, 3 H); MS:(ES⁺) 338 m/z (M + 1)⁺C₁₈H₁₆N₃O₂S requires 338 |
| 212 | 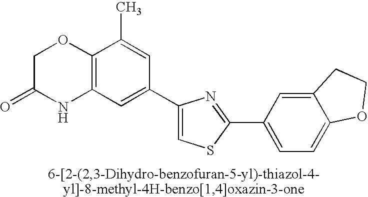 | ¹H NMR (400 MHz,DMSO-_{d6}) δ 10.63 (s, 1 H),7.80 (br s, 1 H), 7.74 (s, 1 H),7.67 (dd, J = 1.6, 8.4 Hz,1 H), 7.37 (d, J = 8.4 Hz,2 H), 6.81 (d, J = 8.4 Hz,1 H), 4.56-4.52 (m, 4 H),3.26-3.17 (m, 2 H), 2.15 (s,3 H); MS: (ES⁺) 365 m/z(M + 1)⁺ C₂₀H₁₇N₂O₃Srequires 365 |
| 213 | 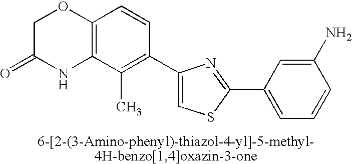 | . ¹H NMR (400 MHz,DMSO-_{d6}) δ 10.63 (s, 1 H),7.56 (s, 1 H), 7.13 (t, J = 2.0Hz, 1 H), 7.08-6.98 (m, 3 H),6.84 (d, J = 8.4 Hz, 1 H),6.60-6.56 (m, 1 H), 4.48 (s,2 H), 2.23 (s, 3 H); MS:(ES⁺) 338 m/z (M + 1)⁺C₁₈H₁₆N₃O₂S requires 338 |
| 214 | 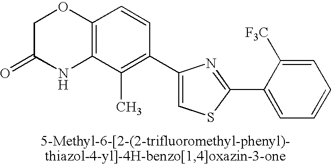 | ¹H NMR (400 MHz,DMSO-_{d6}) δ 10.23 (s, 1 H),7.87 (d, J = 7.6 Hz, 1 H),7.80 (s, 1 H), 7.75-7.66 (m,3 H), 7.60 (d, J = 7.6 Hz,1 H), 6.84 (d, J = 8.4 Hz,1 H), 4.48 (s, 2 H), 2.20 (s,3 H); MS: (ES⁺) 391 m/z(M + 1)⁺ C₁₉H₁₄F₃N₂O₂Srequires 391 |
| 215 | 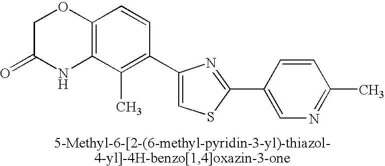 | ¹H NMR (400 MHz,DMSO-_{d6}) δ 10.26 (s, 1 H),8.94 (d, J = 2.4 Hz, 1 H),8.13 (dd, J = 2.4, 8.0 Hz,1 H), 7.70 (s, 1 H), 7.33 (d, J =8.0 Hz, 1 H), 7.10 (d, J =8.4 Hz, 1 H), 6.84 (d, J = 8.4Hz, 1 H), 4.48 (s, 2 H), 2.41(s, 3 H), 2.23 (s, 3 H); MS:(ES⁺) 338 m/z (M + 1)⁺C₁₈H₁₆N₃O₂S requires 338 |
| 216 | 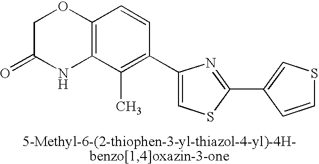 | . ¹H NMR (400 MHz,DMSO-_{d6}) δ 10.24 (s, 1 H),8.10-8.08 (m, 1 H), 7.63 (dd,J = 3.2, 5.2 Hz, 1 H), 7.55 (s,1 H), 7.52 (dd, J = 1.2, 4.8Hz, 1 H), 7.06 (d, J = 8.4 Hz,1 H), 6.83 (d, J = 8.4 Hz,1 H), 4.48 (s, 2 H), 2.21 (s,3 H); MS: (ES⁺) 329 m/z(M + 1)⁺ C₁₆H₁₃N₂O₂S₂requires 329 |
| 217 | 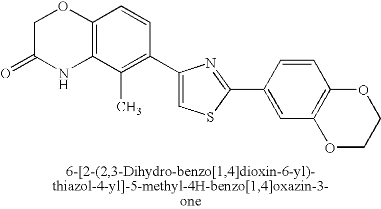 | ¹H NMR (400 MHz,DMSO-_{d6}) δ 10.24 (s, 1 H),7.55 (s, 1 H), 7.37-7.34 (m,2 H), 7.07 (d, J = 8.0 Hz,1 H), 6.90 (d, J = 7.6 Hz,1 H), 6.83 (d, J = 8.0 Hz,1 H), 4.48 (s, 2 H), 4.23 (s,4 H), 2.22 (s, 3 H); MS: (ES⁺)381 m/z (M + 1)⁺C₂₀H₁₇N₂O₄S requires 381 |
| 218 | 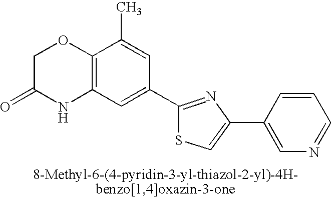 | ¹H NMR (400 MHz,DMSO-_{d6}) δ 10.72 (s, 1 H),9.15 (br s, 1 H), 8.49 (dd, J =1.6, 8.4 Hz, 1 H), 8.30 (dt, J =1.2, 8.0 Hz, 1 H), 8.20 (s,1 H), 7.46-7.39 (m, 3 H), 4.61(s, 2 H), 2.11 (s, 3 H); MS:(ES⁺) 324 m/z (M + 1)⁺C₁₇H₁₄N₃O₂S requires 324 |
| 219 | 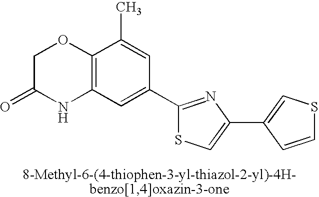 | ¹H NMR (400 MHz,DMSO-_{d6}) δ 10.73 (s, 1 H),7.87 (t, J = 1.2 Hz, 1 H),7.85 (s, 1 H), 7.57-7.55 (m,2 H), 7.39-7.34 (m, 2 H), 4.60(s, 2 H), 2.17 (s, 3 H); MS:(ES⁺) 329 m/z (M + 1)⁺C₁₆H₁₃N₂O₂S₂ requires 329 |
| 220 | 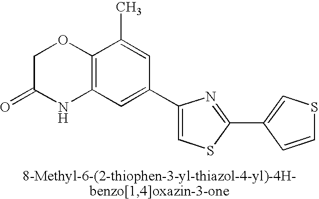 | . ¹H NMR (400 MHz,DMSO-_{d6}) δ 10.67 (s, 1 H),8.09 (dd, J = 1.2, 2.8 Hz,1 H), 7.79 (s, 1 H), 7.66 (dd,J = 3.2, 5.2 Hz, 1 H), 7.54(dd, J = 1.2, 5.2 Hz, 1 H),7.38 (br s, 1 H), 7.33 (br s,1 H), 4.55 (s, 2 H), 2.15 (s,3 H); MS: (ES⁺) 329 m/z(M + 1)⁺ C₁₆H₁₃N₂O₂S₂requires 329 |
| 221 | 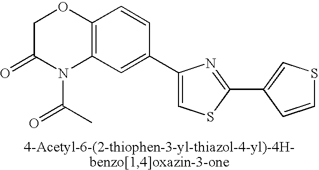 | ¹H NMR (400 MHz,DMSO-_{d6}) δ 8.22-8.19 (m,2 H), 8.01 (s, 1 H), 7.96-7.55(m, 3 H), 7.23 (d, J = 8.4 Hz,1 H), 4.77 (s, 2 H), 2.50 (s,3 H); MS: (ES⁺) 357 m/z(M + 1)⁺ C₁₇H₁₃N₂O₃S₂requires 357 |
| 222 | 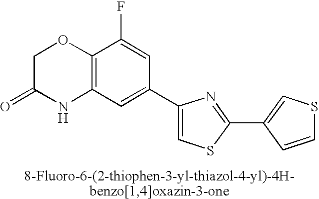 | ¹H NMR (400 MHz,DMSO-_{d6}) δ 8.19 (dd, J =1.2, 2.8 Hz, 1 H), 8.02 (s,1 H), 7.62 (dd, J = 1.6, 5.2Hz, 1 H), 7.54 (dd, J = 1.6,11.6 Hz, 1 H), 7.44 (br s,1 H), 4.71 (s, 1 H); MS:(ES⁺) 333 m/z (M + 1)⁺C₁₅H₁₀FN₂O₂S₂ requires333 |
| 223 | 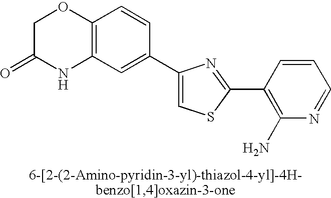 | . ¹H NMR (400 MHz,DMSO-_{d6}) δ 10.79 (s, 1 H),8.04 (dd, J = 1.6, 8.4 Hz,1 H), 7.92 (dd, J = 1.6, 8.0Hz, 1 H), 7.89 (s, 1 H), 7.60(s, 2 H), 4.50-7.42 (m, 2 H),6.97 (d, J = 8.8 Hz, 1 H),6.61 (dd, J = 3.2, 7.6 Hz,1 H), 4.54 (s, 2 H); MS: (ES⁺)325 m/z (M + 1)⁺C₁₆H₁₃N₄O₂S requires 325 |
| 224 | 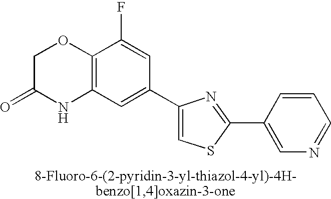 | . ¹H NMR (400 MHz,DMSO-_{d6}) δ 9.20 (d, J = 1.8Hz, 1 H), 8.70 (dd, J = 1.2,2.4 Hz, 1 H), 8.36 (dt, J =1.8, 7.8 Hz, 1 H), 8.20 (s,1 H), 7.60-758 (m, 2 H), 7.49(s, 1 H), 4.72 (s, 2 H); MS:(ES⁺) 328 m/z (M + 1)⁺C₁₆H₁₁FN₃O₂S requires 328 |
| 225 | 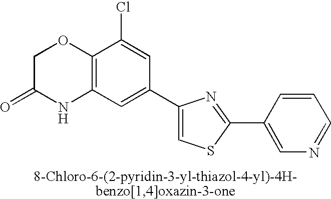 | ¹H NMR (400 MHz,DMSO-_{d6}) δ 10.98 (s, 1 H),9.20 (d, J = 1.8 Hz, 1 H),8.71 (dd, J = 1.2, 4.8 Hz,1 H), 8.36 (dt, J = 1.8, 7.8Hz, 1 H), 8.23 (s, 1 H), 7.76(d, J = 2.4 Hz, 1 H), 7.60-7.57(m, 2 H), 4.76 (s, 2 H);MS: (ES⁺) 344 m/z (M + 1)⁺C₁₆H₁₁ClN₃O₂S requires344 |
| 226 | 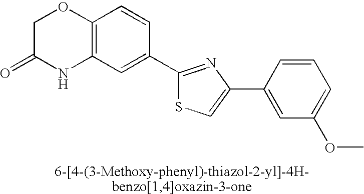 | ¹H NMR (400 MHz,DMSO-_{d6}) δ 10.77 (s, 1 H),8.07 (s, 1 H), 7.55-7.47 (m,4 H), 7.31 (t, J = 8.0 Hz,1 H), 7.00 (d, J = 8.4 Hz,1 H), 6.88 (dd, J = 2.0, 8.4Hz, 1 H), 4.59 (s, 2 H), 3.76(s, 3 H); MS: (ES⁺) 339 m/z(M + 1)⁺ C₁₈H₁₅N₂O₃Srequires 339 |
| 227 | 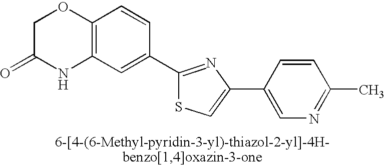 | ¹H NMR (400 MHz,DMSO-_{d6}) δ 9.09 (d, J = Hz,1 H), 8.23 (dd, J = 2.0, 8.0Hz, 1 H), 8.21 (s, 1 H), 7.62(d, J = 2.0 Hz, 1 H), 7.57(dd, J = 2.0, 8.4 Hz, 1 H),7.36 (d, J = 8.0 Hz, 1 H),7.08 (d, J = 8.4 Hz, 1 H),4.68 (s, 2 H), 2.52 (s, 3 H);MS: (ES⁺) 324 m/z (M + 1)⁺C₁₇H₁₄N₃O₂S requires 324 |
| 228 | 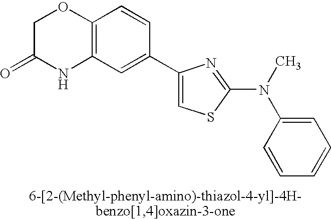 | ¹H NMR (400 MHz, CDCl₃)δ 7.62 (s, 1 H), 7.45 (d, J =1.2 Hz, 2 H), 7.30 (s, 2 H),7.40 (d, J = 2.0 Hz, 1 H),7.36 (d, J = 2.0 Hz, 1 H),6.98 (d, J = 8.4 Hz, 1 H),6.50 (s, 1 H), 4.64 (s, 2 H),3.62 (s, 3 H). MS: (ES⁺)338 m/z (M + 1)⁺C₁₈H₁₅N₃O₂S requires 338 |
| 229 | 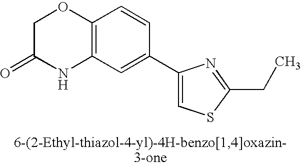 | ¹H NMR (400 MHz, CDCl₃)δ 7.54 (s, 1 H), 7.45 (dd, J =8.4 Hz, 2.0 Hz, 1 H), 7.40(d, J = 2.0 Hz, 1 H), 7.24 (s,1 H), 7.01 (d, J = 8.4 Hz,1 H), 4.65 (s, 2 H), 3.08 (q, J =7.6 Hz, 2 H), 1.44 (t, J =7.6 Hz). MS: (ES⁺) 261m/z (M + 1)⁺ C₁₃H₁₂N₂O₂Srequires 261 |
| 230 | 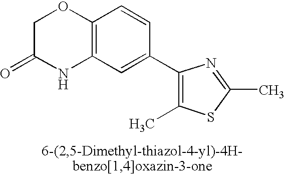 | ¹H NMR (400 MHz, CDCl₃)δ 8.60 (s, 1 H), 7.19 (d, J =1.6 Hz, 1 H), 7.10 (dd, J =8.4 Hz, 2.0 Hz, 1 H), 7.05 (d,J = 8.4 Hz, 1 H), 4.67 (s,2 H), 2.88 (s, 3 H), 2.52 (s,3 H). MS: (ES⁺) 261 m/z(M + 1)⁺ C₁₃H₁₂N₂O₂Srequires 261 |
| 231 | 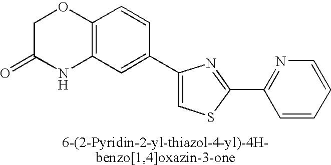 | ¹H NMR (400 MHz,DMSO-_{d6}) δ 10.80 (s, 1 H),8.66 (d, J = 4.4 Hz, 1 H),8.20 (d, J = 7.6 Hz, 1 H),8.08 (s, 1 H), 8.06-8.00 (m,1 H), 7.63 (d, J = 2.0 Hz,1 H), 7.59 (dd, J = 8.4 Hz,2.0 Hz, 1 H), 7.56-7.52 (m,1 H), 7.05 (d, J = 8.4 Hz,1 H), 4.63 (s, 2 H). MS:(ES⁺) 310 m/z (M + 1)⁺C₁₆H₁₁N₃O₂S requires 310 |
| 232 | 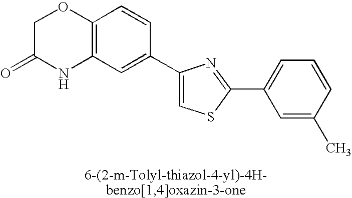 | ¹H NMR (400 MHz, CDCl₃)δ 7.86 (s, 1 H), 7.80 (d, J =7.6 Hz, 1 H), 7.57-7.52 (m,3 H), 7.39-7.33 (m, 2 H), 7.05(d, J = 8.0 Hz, 1 H), 4.67 (s,2 H), 2.45 (s, 3 H). MS:(ES⁺) 323 m/z (M + 1)⁺C₁₈H₁₄N₂O₂S requires 323 |
| 233 | 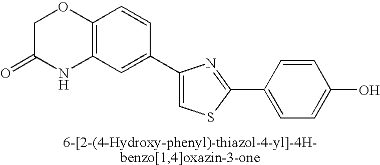 | ¹H NMR (400 MHz,DMSO-_{d6}) δ 10.80 (s, 1 H),10.00 (s, 1 H), 7.85 (s, 1 H),7.82 (d, J = 8.4 Hz, 2 H),7.61-7.59 (m, 1 H), 7.54 (dd,J = 8.4 Hz, 1.6 Hz, 1 H),7.02 (d, J = 1.2 Hz, 1 H),6.90 (d, J = 8.4 Hz, 2 H),4.62 (s, 2 H). MS: (ES⁺)325 m/z (M + 1)⁺C₁₇H₁₂N₂O₃S requires 325 |
| 234 | 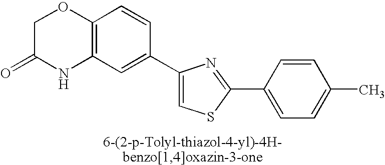 | ¹H NMR (400 MHz, CDCl₃)δ 7.90 (d, J = 8.0 Hz, 1 H),7.62 (s, 1 H), 7.56-7.52 (m,2 H), 7.36 (s, 1 H), 7.28 (s,1 H), 7.04 (d, J = 8.4 Hz,1 H), 4.68 (s, 2 H), 2.42 (s,3 H). MS: (ES⁺) 323 m/z(M + 1)⁺ C₁₈H₁₄N₂O₂Srequires 323 |
| 235 | 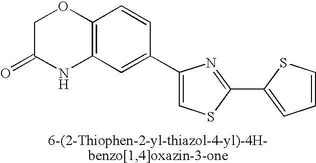 | ¹H NMR (400 MHz, CDCl₃)δ 7.64 (s, 1 H), 7.56 (d, J =3.2 Hz, 1 H), 7.52-7.48 (m,2 H), 7.42 (d, J = 5.2 Hz,1 H), 7.31 (s, 1 H), 7.11 (t, J =4.0 Hz, 1 H), 7.03 (d, J =8.0 Hz, 1 H), 4.68 (s, 2 H).MS: (ES⁺) 315 m/z (M + 1)⁺C₁₅H₁₀N₂O₂S₂ requires 315 |
| 236 | 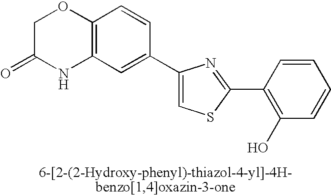 | ¹H NMR (400 MHz,DMSO-_{d6}) δ 11.30 (s, 1 H),10.80 (s, 1 H), 8.18 (dd, J =3.6 Hz, 1.6 Hz, 1 H), 7.98 (s,1 H), 7.64 (d, J = 1.6 Hz,1 H), 7.56 (dd, J = 8.4 Hz,2.0 Hz, 1 H), 7.36-7.30 (m,1 H), 7.08-6.96 (m, 3 H), 4.62(s, 2 H). MS: (ES⁺) 325 m/z(M + 1)⁺ C₁₇H₁₂N₂O₃Srequires 325 |
| 237 | 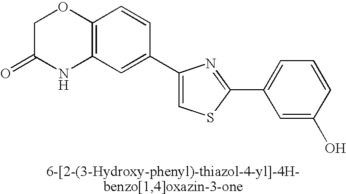 | ¹H NMR (400 MHz,DMSO-_{d6}) δ 10.70 (s, 1 H),9.80 (s, 1 H), 8.00 (s, 1 H),7.62 (d, J = 2.0 Hz, 1 H),7.56 (dd, J = 8.4 Hz, 2.4 Hz,1 H), 7.42-7.38 (m, 2 H),7.36-7.30 (m, 1 H), 7.04 (d, J =8.4 Hz, 1 H), 6.92-6.88 (m,1 H), 4.62 (s, 2 H). MS:(ES⁺) 325 m/z (M + 1)⁺C₁₇H₁₂N₂O₃S requires 325 |
| 238 | 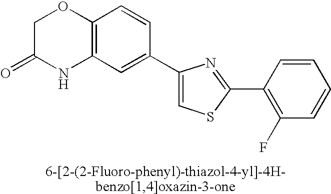 | ¹H NMR (400 MHz,DMSO-_{d6}) δ 10.80 (s, 1 H),8.32 (td, J = 8.0 Hz, 2.0 Hz,1 H), 8.14 (s, 1 H), 7.66 (d, J =2.0 Hz, 1 H), 7.62-7.55 (m,2 H), 7.50-7.40 (m, 2 H), 7.05(d, J = 8.4 Hz, 1 H), 4.64 (s,2 H). MS: (ES⁺) 327 m/z(M + 1)⁺ C₁₇H₁₁FN₂O₂Srequires 327 |
| 239 | 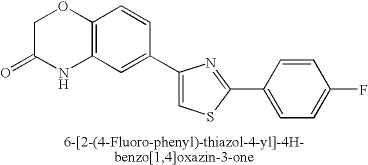 | . ¹H NMR (400 MHz,DMSO-_{d6}) δ 10.80 (s, 1 H),8.08-8.02 (m, 2 H), 7.98 (s,1 H), 7.62 (d, J = 2.0 Hz,1 H), 7.56 (dd, J = 2.0 Hz,2 H), 7.42-7.36 (m, 2 H), 7.04(d, J = 8.0 Hz, 1 H), 4.62 (s,2 H). MS: (ES⁺) 327 m/z(M + 1)⁺ C₁₇H₁₁FN₂O₂Srequires 327 |
| 240 | 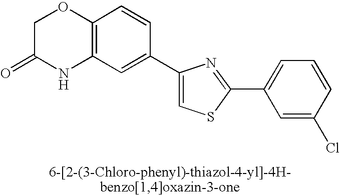 | ¹H NMR (400 MHz,DMSO-_{d6}) δ 10.80 (s, 1 H),8.08 (s, 1 H), 8.06-8.04 (m,1 H), 7.96-7.92 (m, 1 H), 7.65(d, J = 2.0 Hz, 1 H), 7.60-7.56(m, 3 H), 7.04 (d, J =8.4 Hz, 1 H), 4.64 (s, 2 H).MS: (ES⁺) 343 m/z (M + 1)⁺C₁₇H₁₁ClN₂O₂S requires 343 |
| 241 | 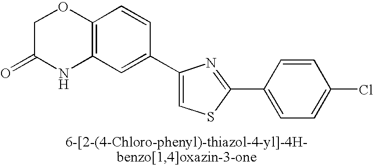 | ¹H NMR (400 MHz,DMSO-_{d6}) δ 10.80 (s, 1 H),8.05-8.00 (m, 3 H), 7.65-7.61(m, 3 H), 7.58 (dd, J =8.0 Hz, 2.0 Hz, 1 H), 7.04 (d,J = 8.4 Hz, 1 H), 4.64 (s,2 H). MS: (ES⁺) 343 m/z(M + 1)⁺ C₁₇H₁₁ClN₂O₂Srequires 343 |
| 242 | 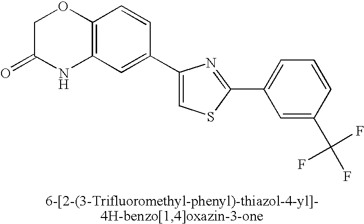 | ¹H NMR (400 MHz,DMSO-_{d6}) δ 10.80 (s, 1 H),8.31 (s, 1 H), 8.28 (d, J = 8.0Hz, 1 H), 8.12 (s, 1 H), 7.94-7.88(m, 1 H), 7.82-7.77 (m,1 H), 7.66 (d, J = 2.0 Hz,1 H), 7.59 (dd, J = 8.0 Hz,2.0 Hz, 1 H), 7.05 (d, J = 8.4Hz, 1 H), 4.64 (s, 2 H). MS:(ES⁺) 343 m/z (M + 1)⁺C₁₇H₁₁ClN₂O₂S requires 343 |
| 243 | 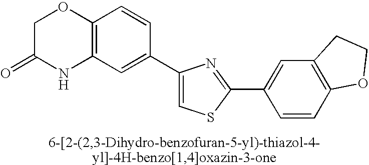 | ¹H NMR (400 MHz,DMSO-_{d6}) δ 10.80 (s, 1 H),7.90-7.86 (m, 2 H), 7.76 (d, J =8.4 Hz, 1 H), 7.62 (s, 1 H),7.55 (d, J = 8.4 Hz, 1 H),7.02 (d, J = 8.4 Hz, 1 H),6.90 (d, J = 8.4 Hz, 1 H),4.66-4.60 (m, 3 H), 3.28 (t, J =8.4 Hz, 2 H). MS: (ES⁺)351 m/z (M + 1)⁺C₁₉H₁₄N₃O₂S requires 351 |
| 244 | 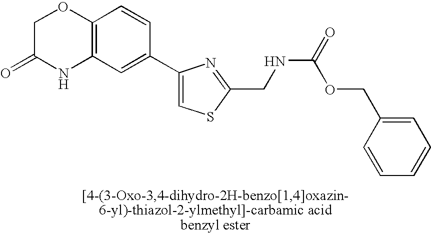 | . ¹H NMR (400 MHz,DMSO-_{d6}) δ 10.80 (s, 1 H),8.24 (t, J = 6.0 Hz, 1 H),7.84 (s, 1 H), 7.51 (d, J = 2.0Hz, 1 H), 7.46 (dd, J = 8.0Hz, 2.0 Hz, 1 H), 7.40-7.30(m, 4 H), 7.00 (d, J = 8.4 Hz,1 H), 5.08 (s, 2 H), 4.60 (s,2 H), 4.56 (d, J = 6.4 Hz,2 H). MS: (ES⁺) 396 m/z(M + 1)⁺ C₂₀H₁₇N₃O₄Srequires 396 |
| 245 | 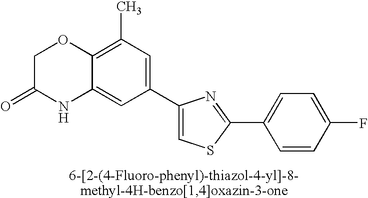 | ¹H NMR (400 MHz,DMSO-_{d6}) δ 10.80 (s, 1 H),8.08-8.03 (m, 2 H), 7.96 (s,1 H), 7.50-7.44 (m, 2 H),7.42-7.36 (m, 2 H), 4.64 (s,2 H), 2.24 (s, 3 H). MS:(ES⁺) 341 m/z (M + 1)⁺C₁₈H₁₃FN₂O₂S requires 341 |
| 246 | 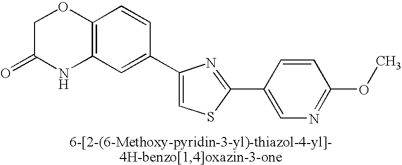 | ¹H NMR (400 MHz,DMSO-_{d6}) δ 10.80 (s, 1 H),8.06 (d, J = 2.8 Hz, 1 H),7.99 (dd, J = 9.6 Hz, 2.4 Hz,1 H), 7.86 (s, 1 H), 7.58 (d, J =2.0 Hz, 1 H), 7.54 (dd, J =8.4 Hz, 2.0 Hz, 1 H), 7.02 (d,J = 8.4 Hz, 1 H), 6.50 (d, J =9.2 Hz, 1 H), 4.62 (s, 2 H),3.32 (s, 3 H). MS: (ES⁺)340 m/z (M + 1)⁺C₁₇H₁₃N₃O₃S requires 340 |
| 247 | 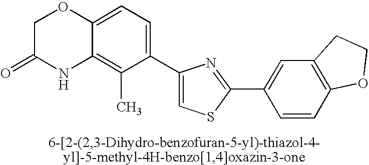 | ¹H NMR (400 MHz,DMSO-_{d6}) δ 10.40 (s, 1 H),7.86-7.84 (m, 1 H), 7.72 (dd,J = 8.4 Hz, 2.0 Hz, 1 H),7.58 (s, 1 H), 7.16 (d, J = 8.4Hz, 1 H), 6.91 (d, J = 8.0 Hz,1 H), 6.88 (d, J = 8.4 Hz,1 H), 4.62 (t, J = 8.8 Hz,2 H), 4.56 (s, 2 H), 3.26 (t, J =8.8 Hz, 2 H), 2.24 (s, 3 H).MS: (ES⁺) 365 m/z (M + 1)⁺C₂₀H₁₆N₂O₃S requires 365 |
| 248 | 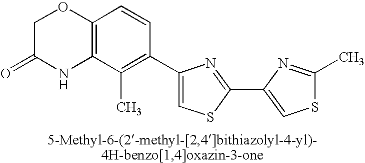 | ¹H NMR (400 MHz,DMSO-_{d6}) δ 10.40 (s, 1 H),8.10 (s, 1 H), 7.68 (s, 1 H),7.16 (d, J = 8.4 Hz, 1 H),6.92 (d, J = 8.4 Hz, 1 H),4.56 (s, 2 H), 2.74 (s, 3 H),2.28 (s, 3 H). MS: (ES⁺)344 m/z (M + 1)⁺C₁₆H₁₃N₃O₂S requires 344 |
| 249 | 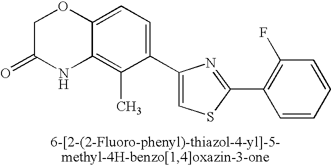 | ¹H NMR (400 MHz,DMSO-_{d6}) δ 10.40 (s, 1 H),8.26 (td, J = 8.0 Hz, 7.6 Hz,1.6 Hz, 1 H), 7.88 (s, 1 H),7.60-7.52 (m, 1 H), 7.48-7.36(m, 2 H), 7.20 (d, J =8.0 Hz, 1 H), 6.94 (d, J = 8.4Hz, 1 H), 4.58 (s, 2 H), 2.32(s, 3 H). MS: (ES⁺) 341 m/z(M + 1)⁺ C₁₈H₁₃FN₂O₂Srequires 341 |
| 250 | 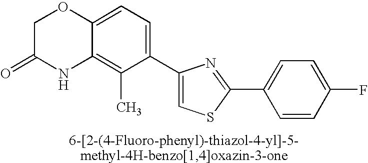 | ¹H NMR (400 MHz,DMSO-_{d6}) δ 10.40 (s, 1 H),8.05-8.00 (m, 2 H), 7.74 (s,1 H), 7.40-7.33 (m, 2 H), 7.17(d, J = 8.0 Hz, 1 H), 6.92 (d,J = 8.4 Hz, 1 H), 4.56 (s,2 H), 2.32 (s, 3 H). MS:(ES⁺) 341 m/z (M + 1)⁺C₁₈H₁₃FN₂O₂S requires 341 |
| 251 | 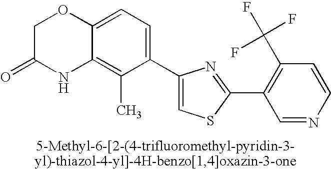 | ¹H NMR (400 MHz,DMSO-_{d6}) δ 9.10 (s, 1 H),9.00 (d, J = 5.2 Hz, 1 H),8.00-7.97 (m, 2 H), 7.16 (d, J =8.4 Hz, 1 H), 6.93 (d, J =8.4 Hz, 1 H), 4.56 (s, 2 H),2.28 (s, 3 H). MS: (ES⁺)392 m/z (M + 1)⁺C₁₈H₁₂F₃N₃O₂S requires 392 |
| 252 | 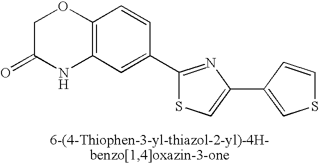 | ¹H NMR (400 MHz,DMSO-_{d6}) δ 10.85 (s, 1 H),7.98-7.94 (m, 2 H), 7.66-7.63(m, 2 H), 7.58 (d, J =2.0 Hz, 1 H), 7.55 (dd, J =8.4 Hz, 2.0 Hz, 1 H), 7.08 (d,J = 8.4 Hz, 1 H), 4.67 (s,2 H). MS: (ES⁺) 315 m/z(M + 1)⁺ C₁₅H₁₀N₂O₂S₂requires 315 |
| 253 | 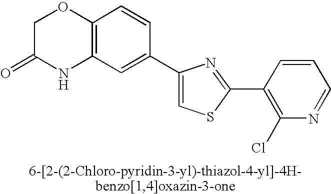 | ¹H NMR (400 MHz,DMSO-_{d6}) δ 10.80 (s, 1 H),8.70 (dd, J = 7.6 Hz, 2.0 Hz,1 H), 8.56 (dd, J = 4.8 Hz,2.0 Hz, 1 H), 8.25 (s, 1 H),7.67 (dd, J = 4.8 Hz, 4.8 Hz,1 H), 7.64 (d, J = 2.0 Hz,1 H), 7.61 (dd, J = 8.4 Hz,2.0 Hz, 1 H), 7.05 (d, J = 8.0Hz, 1 H), 4.63 (s, 2 H). MS:(ES⁺) 344 m/z (M + 1)⁺C₁₆H₁₀ClN₃O₂S requires 344 |
| 254 | 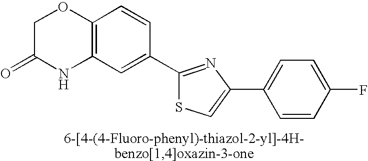 | . ¹H NMR (400 MHz,DMSO-_{d6}) δ 10.80 (s, 1 H),8.12 (s, 1 H), 8.08-8.04 (m,2 H), 7.61 (d, J = 2.4 Hz,1 H), 7.56 (dd, J = 8.4 Hz,2.4 Hz, 1 H), 7.35-7.28 (m,2 H), 7.08 (d, J = 8.4 Hz,1 H), 4.67 (s, 2 H). MS:(ES⁺) 327 m/z (M + 1)⁺C₁₇H₁₁FN₂O₂S requires 327 |
| 255 | 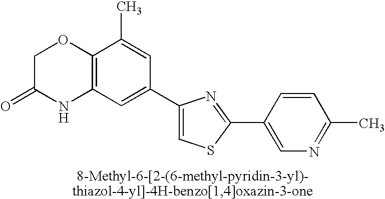 | ¹H NMR (400 MHz,DMSO-_{d6}) δ 10.80 (s, 1 H),9.09 (d, J = 2.0 Hz, 1 H),8.34-8.28 (m, 1 H), 8.04 (s,1 H), 7.52-7.44 (m, 3 H), 4.64(s, 2 H), 2.58 (s, 3 H), 2.26 (s,3 H). MS: (ES⁺) 338 m/z(M + 1)⁺ C₁₈H₁₅N₃O₂Srequires 338 |
| 256 | 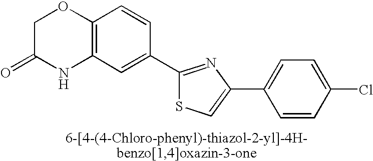 | ¹H NMR (400 MHz,DMSO-_{d6}) δ 10.80 (s, 1 H),8.19 (s, 1 H), 8.09-8.02 (m,2 H), 7.62 (d, J = 2.0 Hz,1 H), 7.58-7.52 (m, 3 H), 7.08(d, J = 8.4 Hz, 1 H), 4.68 (s,2 H). MS: (ES⁺) 343 m/z(M + 1)⁺ C₁₇H₁₁ClN₂O₂Srequires 343 |
| 257 | 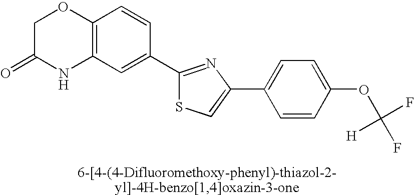 | . ¹H NMR (400 MHz,DMSO-_{d6}) δ 10.80 (s, 1 H),8.13 (s, 1 H), 8.09-8.05 (m,2 H), 7.62 (d, J = 2.0 Hz,1 H), 7.57 (d, J = 8.4 Hz, 2.4Hz, 1 H), 7.32 (t, J = 74.0Hz, 1 H), 7.29 (d, J = 8.8 Hz,2 H), 7.08 (d, J = 8.4 Hz,1 H), 4.68 (s, 2 H). MS:(ES⁺) 375 m/z (M + 1)⁺C₁₈H₁₂F₂N₂O₃S requires 375 |
| 258 | 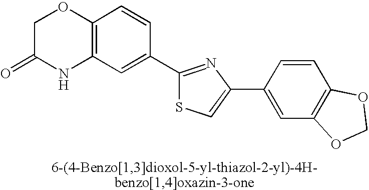 | ¹H NMR (400 MHz,DMSO-_{d6}) δ, 10.80 (s, 1 H),7.98 (s, 1 H), 7.62 (d, J = 2.0Hz, 1 H), 7.60-7.53 (m, 3 H),7.08 (d, J = 8.4 Hz, 1 H),7.04-7.01 (m, 1 H), 6.08 (s,2 H), 4.68 (s, 2 H). MS:(ES⁺) 353 m/z (M + 1)⁺C₁₈H₁₂N₂O₄S requires 353 |
| 259 | 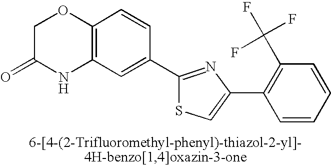 | ¹H NMR (400 MHz,DMSO-_{d6}) δ, 10.80 (s, 1 H),7.88 (d, J = 8.0 Hz, 1 H),7.80 (s, 1 H), 7.78-7.64 (m,3 H), 7.56 (d, J = 2.0 Hz,1 H), 7.52 (dd, J = 8.4 Hz,2.0 Hz, 1 H), 7.08 (d, J = 8.4Hz, 1 H), 4.66 (s, 2 H). MS:(ES⁺) 377 m/z (M + 1)⁺C₁₈H₁₁F₃N₂O₂S requires 377 |
| 260 | 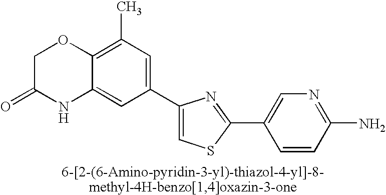 | ¹H NMR (400 MHz,DMSO-_{d6}) δ, 10.70 (s, 1 H),8.55 (d, J = 2.4 Hz, 1 H),7.91 (dd, J = 8.8 Hz, 2.8 Hz,1 H), 7.77 (s, 1 H), 7.48-7.42(m, 2 H), 6.60-6.53 (m, 3 H),4.64 (s, 2 H), 2.24 (s, 3 H).MS: (ES⁺) 339 m/z (M + 1)⁺C₁₇H₁₄N₄O₂S requires 339 |
| 261 | 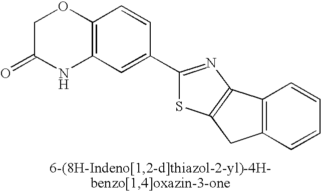 | . ¹H NMR (400 MHz,DMSO-_{d6}) δ, 10.80 (s, 1 H),7.70 (d, J = 7.2 Hz, 1 H),7.63 (d, J = 2.0 Hz, 1 H),7.60 (d, J = 7.6 Hz, 1 H),7.56 (dd, J = 8.4 Hz, 2.0 Hz,1 H), 7.43-7.38 (m, 1 H),7.32-7.27 (m, 1 H), 7.08 (d, J =8.0 Hz, 1 H), 4.67 (s, 2 H),4.00 (s, 2 H). MS: (ES⁺)321 m/z (M + 1)⁺C₁₈H₁₂N₂O₂S requires 321 |
| 262 | 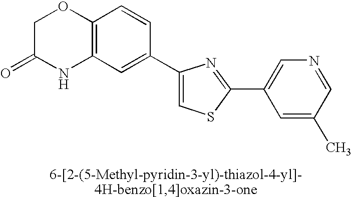 | ¹H NMR (400 MHz,DMSO-_{d6}) δ, 10.80 (s, 1 H),9.00 (d, J = 2.0 Hz, 1 H),8.55 (d, J = 1.6 Hz, 1 H),8.19-8.17 (m, 1 H), 8.08 (s,1 H), 7.65 (d, J = 1.6 Hz,1 H), 7.58 (dd, J = 8.4 Hz,2.0 Hz, 1 H), 7.05 (d, J = 8.4Hz, 1 H), 4.67 (s, 2 H), 2.40(s, 3 H). MS: (ES⁺) 324 m/z(M + 1)⁺ C₁₇H₁₃N₃O₂Srequires 324 |
| 263 | 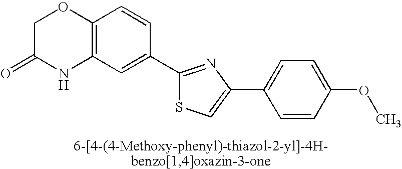 | ¹H NMR (400 MHz,DMSO-_{d6}) δ, 10.80 (s, 1 H),7.98-7.92 (m, 3 H), 7.61 (d, J =2.0 Hz, 1 H), 7.54 (dd, J =8.0 Hz, 2.0 Hz, 1 H), 7.09-7.02(m, 3 H), 4.67 (s, 2 H),3.80 (s, 3 H). MS: (ES⁺)339 m/z (M + 1)⁺C₁₈H₁₄N₂O₃S requires 339 |
| 264 | 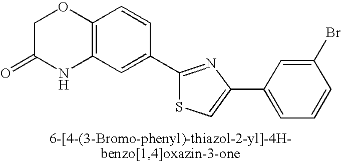 | . ¹H NMR (400 MHz,DMSO-_{d6}) δ, 10.80 (s, 1 H),8.28 (s, 1 H), 8.24-8.22 (m,1 H), 8.03 (d, J = 7.6 Hz,1 H), 7.65 (d, J = 2.0 Hz,1 H), 7.60-7.56 (m, 2 H), 7.45(t, J = 8.0 Hz, 1 H), 7.08 (d,J = 8.4 Hz, 1 H), 4.68 (s,2 H). MS: (ES⁺) 388 m/z(M + 1)⁺ C₁₇H₁₁BrN₂O₂Srequires 388 |
| 265 | 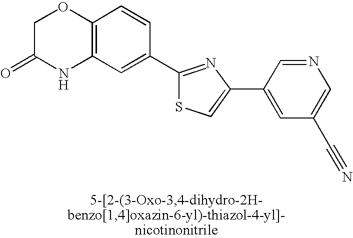 | ¹H NMR (400 MHz,DMSO-_{d6}) δ, 10.80 (s, 1 H),9.50 (d, J = 2.0 Hz, 1 H),9.02 (d, J = 2.0 Hz, 1 H),8.87 (t, J = 2.0 Hz, 1 H),8.47 (s, 1 H), 7.65-7.62 (m,2 H), 7.10 (d, J = 9.2 Hz,1 H), 4.68 (s, 2 H). MS:(ES⁺) 335 m/z (M + 1)⁺C₁₇H₁₀N₄O₂S requires 335 |
| 266 | 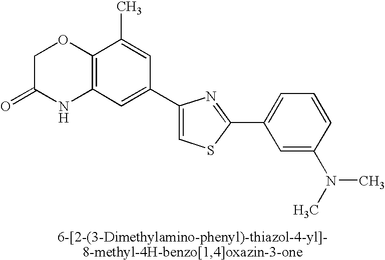 | ¹H NMR (400 MHz,DMSO-_{d6}) δ, 10.80 (s, 1 H),7.92 (s, 1 H), 7.47 (s, 2 H),7.35-7.22 (m, 3 H), 6.87 (dd,J = 8.0 Hz, 2.4 Hz, 1 H),4.64 (s, 2 H), 2.98 (s, 6 H),2.23 (s, 3 H). MS: (ES⁺)366 m/z (M + 1)⁺C₂₀H₁₉N₃O₂S requires 366 |
| 267 | 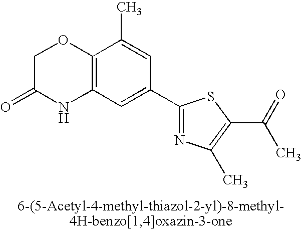 | . ¹H NMR (400 MHz,DMSO-_{d6}) δ, 10.80 (s, 1 H),7.49-7.42 (m, 2 H), 4.68 (s,2 H), 3.28 (s, 3 H), 2.68 (s,3 H), 2.55 (s, 3 H). MS:(ES⁺) 303 m/z (M + 1)⁺C₁₅H₁₄N₂O₃S requires 303 |
| 268 | 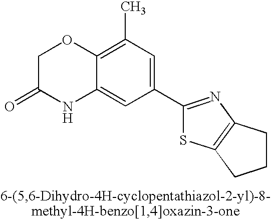 | ¹H NMR (400 MHz,DMSO-_{d6}) δ, 10.80 (s, 1 H),7.32 (d, J = 6.8 Hz, 2 H),4.65 (s, 2 H), 2.90 (t, J = 7.0Hz, 2 H), 2.78 (t, J = 7.0 Hz,2 H), 2.50-2.42 (m, 2 H), 2.20(s, 3 H). MS: (ES⁺) 287 m/z(M + 1)⁺ C₁₅H₁₄N₂O₂Srequires 287 |
| 269 | 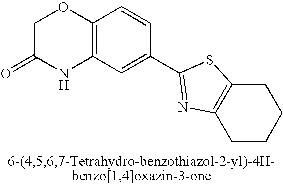 | ¹H NMR (400 MHz,DMSO-_{d6}) δ, 10.80 (s, 1 H),7.47 (d, J = 2.0 Hz, 1 H),7.38 (dd, J = 8.4 Hz, 2.4 Hz,1 H), 7.02 (d, J = 8.4 Hz, 1 H),4.64 (s, 2 H), 2.80-2.66 (m,5 H), 2.57-2.52 (m, 2 H),2.34-2.31 (m, 1 H). MS:(ES⁺) 287 m/z (M + 1)⁺C₁₅H₁₄N₂O₂S requires 287 |
| 270 | 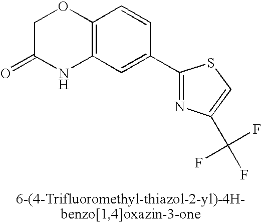 | ¹H NMR (400 MHz,DMSO-_{d6}) δ, 10.80 (s, 1 H),8.50 (d, J = 1.0 Hz, 1 H),7.59-7.57 (m, 1 H), 7.54 (dd,J = 8.4 Hz, 2.0 Hz, 1 H),7.08 (d, J = 8.4 Hz, 1 H),4.68 (s, 2 H). MS: (ES⁺)301 m/z (M + 1)⁺C₁₂H₇F₃N₂O₂S requires 301 |
| 271 | 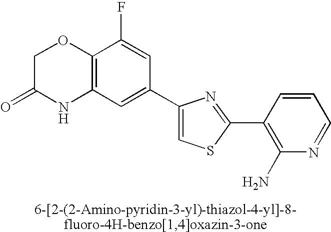 | ¹H NMR (400 MHz,DMSO-_{d6}) δ, 11.00 (s, 1 H),8.36 (s, 2 H), 8.32 (d, J = 7.6Hz, 1 H), 8.19-8.15 (m, 2 H),7.58 (dd, J = 11.6 Hz, 2.0Hz, 1 H), 7.39-7.35 (m, 1 H),6.89 (dd, J = 7.5 Hz, 5.6 Hz,1 H), 4.72 (s, 2 H). MS:(ES⁺) 343 m/z (M + 1)⁺C₁₆H₁₁FN₂O₄S requires 343 |
| 272 | 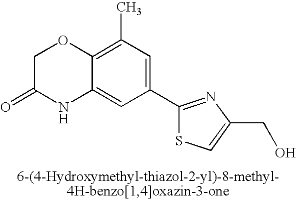 | ¹H NMR (400 MHz,DMSO-_{d6}) δ, 10.80 (s, 1 H),7.40-7.35 (m, 3 H), 4.66 (s,2 H), 4.58 (d, J = 0.8 Hz,2 H), 2.22 (s, 3 H). MS:(ES⁺) 277 m/z (M + 1)⁺C₁₃H₁₂N₂O₃S requires 277 |
| 273 | 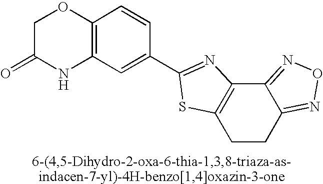 | ¹H NMR (400 MHz,DMSO-_{d6}) δ, 10.80 (s, 1 H),7.62 (d, J = 2.4 Hz, 1 H),7.53 (dd, J = 8.4 Hz, 2.4 Hz,1 H), 7.09 (d, J = 8.4 Hz,1 H), 4.68 (s, 2 H), 3.40-3.30(m, 4 H). MS: (ES⁺) 327m/z (M + 1)⁺ C₁₅H₁₀N₄O₃Srequires 327 |
| 274 | 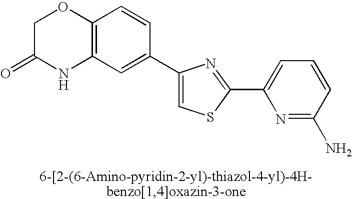 | ¹H NMR (400 MHz,DMSO-_{d6}) δ, 10.80 (s, 1 H),7.80 (s, 1 H), 7.60 (t, J = 8.0Hz, 1 H), 7.44 (dd, J = 16.4Hz, 1.6 Hz, 2 H), 7.36 (d, J =7.2 Hz, 1 H), 6.60 (d, J = 8.4Hz, 1 H), 4.64 (s, 2 H). MS:(ES⁺) 339 m/z (M + 1)⁺C₁₇H₁₄N₄O₂S requires 339 |
| 275 | 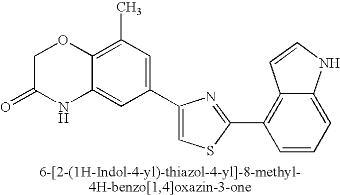 | . ¹H NMR (400 MHz,DMSO-_{d6}) δ, 11.50 (s, 1 H),10.80 (s, 1 H), 7.95 (s, 1 H),7.70 (d, J = 7.2 Hz, 1 H),7.60-7.55 (m, 3 H), 7.51 (d, J =1.2 Hz, 1 H), 7.30-7.28 (m,1 H), 7.22 (t, J = 8.0 Hz,1 H), 4.64 (s, 2 H), 2.26 (s,3 H). MS: (ES⁺) 362 m/z(M + 1)⁺ C₂₀H₁₅N₃O₂Srequires 362 |
| 276 | 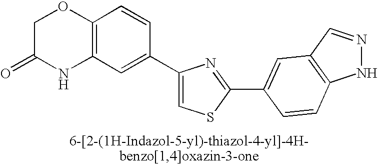 | ¹H NMR (400 MHz,DMSO-_{d6}) δ, 10.80 (s, 1 H),8.44 (s, 1 H), 8.23 (s, 1 H),8.02 (dd, J = 8.8 Hz, 1.4 Hz,1 H), 7.94 (s, 1 H), 7.68 (d, J =9.2 Hz, 1 H), 7.66 (d, J =2.0 Hz, 1 H), 7.58 (dd, J =8.4 Hz, 2.0 Hz, 1 H), 7.04 (d,J = 8.4 Hz, 1 H), 4.63 (s,2 H). MS: (ES⁺) 349 m/z(M + 1)⁺ C₁₅H₁₂N₄O₂Srequires 349 |
| 277 | 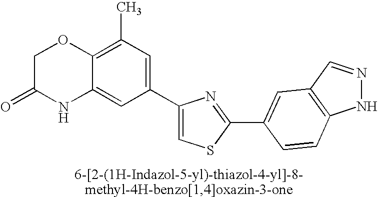 | ¹H NMR (400 MHz,DMSO-_{d6}) δ, 10.80 (s, 1 H),8.44 (s, 1 H), 8.23 (s, 1 H),8.04-7.99 (m, 1 H), 7.89 (s,1 H), 7.68 (d, J = 8.8 Hz,1 H), 7.51-7.47 (m, 2 H), 4.64(s, 2 H), 2.25 (s, 3 H). MS:(ES⁺) 363 m/z (M + 1)⁺C₁₉H₁₄N₄O₂S requires 363 |
| 278 | 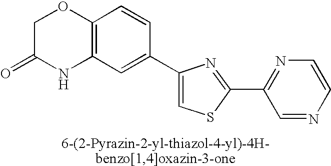 | ¹H NMR (400 MHz,DMSO-_{d6}) δ, 10.80 (s, 1 H),9.38 (d, J = 1.2 Hz, 1 H),8.79-8.74 (m, 2 H), 8.20 (s,1 H), 7.65-7.60 (m, 2 H), 7.07(d, J = 8.4 Hz, 1 H), 4.64 (s,2 H). MS: (ES⁺) 311 m/z(M + 1)⁺ C₁₅H₁₀N₄O₂Srequires 311 |
| 279 | 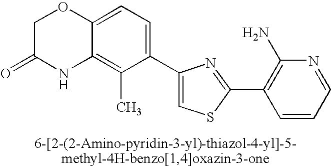 | ¹H NMR (400 MHz,DMSO-_{d6}) δ 10.29 (s, 1 H),8.31 (dd, J = 6.8 Hz, 7.6 Hz,1 H), 8.08 (dd, J = 4.4 Hz,5.6 Hz, 1 H), 7.77 (s, 1 H),7.08 (d, J = 8.4 Hz, 1 H),6.82-6.88 (m, 2 H), 4.49 (s,2 H), 2.20 (s, 3 H). MS: (ES⁺)339 m/z (M + 1)⁺C₁₇H₁₅N₄O₂S requires 339 |
| 280 | 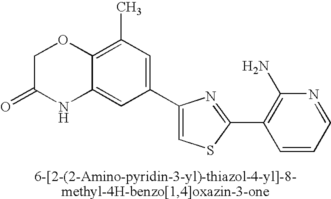 | ¹H NMR (400 MHz,DMSO-_{d6}) δ 10.72 (s, 1 H),8.28 (d, J = 7.6 Hz, 1 H), 8.11(dd, J = 4.0 Hz, 5.2 Hz, 1 H),7.98 (s, 1 H), 7.39 (s, 1 H),7.30 (d, J = 2.0 Hz, 1 Hz),6.86-6.83 (m, 1 H), 4.58 (s,2 H), 2.18 (s, 3 H). MS: (ES⁺)339 m/z (M + 1)⁺C₁₇H₁₅N₄O₂S requires 339 |
| 281 | 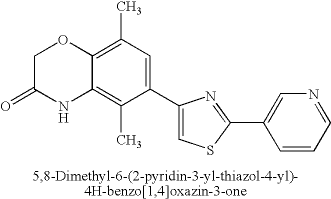 | ¹H NMR (400 MHz,DMSO-_{d6}) δ 10.27 (s, 1 H),9.18 (s, 1 H), 8.70 (d, J =4.0 Hz, 1 H), 8.38 (d, J =8.0 Hz, 1 H), 7.82 (s, 1 H),7.59 (q, J = 5.2 Hz, 1 H), 7.10(s, 1 H), 2.29 (s, 3 H), 2.20 (s,3 H). MS: (ES⁺) 338 m/z(M + 1)⁺ C₁₈H₁₅N₃O₂Srequires 338 |
| 282 | 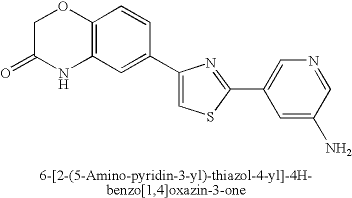 | ¹H NMR (400 MHz,DMSO-_{d6}) δ 10.80 (s, 1 H),8.38 (s, 1 H), 8.04-8.00 (m,1 H), 7.21 (s, 1 H), 7.08 (s,1 H), 6.95 (s, 1 H), 4.62 (s,2 H). MS: (ES⁺) 325 m/z(M + 1)⁺ C₁₆H₁₃N₄O₂Srequires 325 |
| 283 | 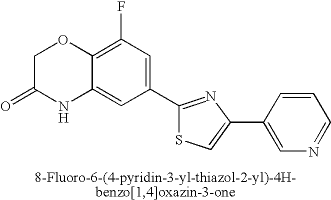 | ¹H NMR (400 MHz,DMSO-_{d6}) δ 11.00 (s, 1 H),9.40-9.10 (m, 1 H), 8.70-8.40(m, 1 H), 8.38 (d, J =8.0 Hz), 8.28 (s, 1 H), 7.54(dd, J = 11.2 Hz, 2.0 Hz,2 H), 7.37-7.47 (m, 1 H), 4.68(s, 2 H). MS: (ES⁺) 328 m/z(M + 1)⁺ C₁₆H₁₁FN₃O₂Srequires 328 |
| 284 | 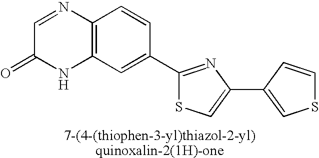 | ¹H NMR (DMSO-d6,400 MHz): 12.44 (s, 1 H),8.14 (d, J = 2.4 Hz, 1 H),8.04 (s, 1 H), 7.94 (t, J = 2.4Hz, 1 H), 7.88 (s, 1 H), 7.83(s, 2 H), 7.60 (m, 2 H). MS(ES⁺) 311, m/z (M + 1) 312,C₁₅H₉N₃OS₂ requires 311 |
| 285 | 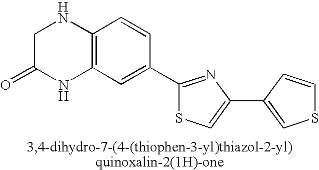 | ¹H NMR (DMSO-d6,400 MHz): 10.33 (s, 1 H),7.82 (dd, J = 2.8, 1.6 Hz,1 H), 7.72 (s, 1 H), 7.55 (m,2 H), 7.33 (m, 2 H), 6.64 (d, J =8.0 Hz, 1 H), 6.45 (s, 1 H),3.76 (s, 2 H). MS (ES⁺) 313,m/z (M + 1) 314,C₁₅H₁₁N₃OS₂ requires 313 |
| 286 | 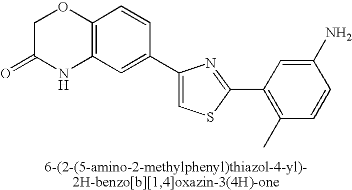 | ¹H NMR (DMSO-d6,400 MHz): 10.78 (s, 1 H),7.97 (s, 1 H), 7.52 (d, J = 2.0Hz, 1 H), 7.50 (dd, J = 8.4,2.0 Hz, 1 H), 7.27 (s, 1 H),7.16 (d, J = 8.4 Hz, 1 H),6.97 (d, J = 8.4 Hz, 1 H),6.85 (d, J = 8.4 Hz, 1 H),4.55 (s, 2 H), 2.48 (s, 3 H).MS (ES⁺) 337, m/z (M + 1)338, C₁₈H₁₅N₃O₂S requires337 |
| 287 | 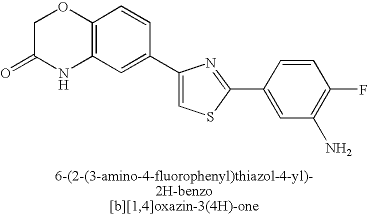 | ¹H NMR (DMSO-d6,400 MHz): 10.75 (s, 1 H),7.85 (s, 1 H), 7.51 (d, J = 2.0Hz, 1 H), 7.48 (dd, J = 8.4,2.0 Hz, 1 H), 7.36 (d, J = 8.4Hz, 1 H), 7.06 (d, J = 8.4 Hz,2 H), 6.97 (d, J = 8.4 Hz,1 H), 4.55 (s, 2 H). MS (ES⁺)341, m/z (M + 1) 342,C₁₇H₁₂FN₃O₂S requires 341 |
| 288 | 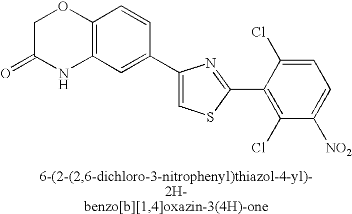 | ¹H NMR (DMSO-d6,400 MHz): 10.74 (s, 1 H),8.32 (s, 1 H), 8.31 (d, J = 8.8Hz, 1 H), 7.97 (d, J = 8.8 Hz,1 H), 7.56 (m, 2 H), 7.04 (d, J =8.8 Hz, 1 H), 4.62 (s, 2 H).MS (ES⁺) 421, m/z (M + 1)422, C₁₇H₉Cl₂N₃O₄Srequires 421 |
| 289 | 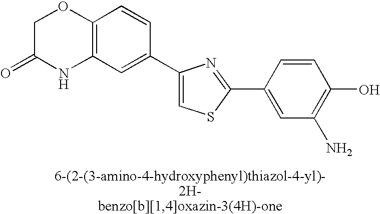 | ¹H NMR (DMSO-d6,400 MHz): 10.82 (s, 1 H),10.10 (s, 1 H), 7.81 (s, 1 H),7.56 (d, J = 2.0 Hz, 1 H),7.53 (dd, J = 8.4, 2.0 Hz,1 H), 7.41 (s, 1 H), 7.24 (d, J =8.4 Hz, 1 H), 7.03 (d, J =8.4 Hz, 1 H), 6.83 (d, J = 8.4Hz, 1 H), 4.62 (s, 2 H). MS(ES⁺) 339, m/z (M + 1) 340,C₁₇H₁₃N₃O₃S requires 339 |
| 290 | 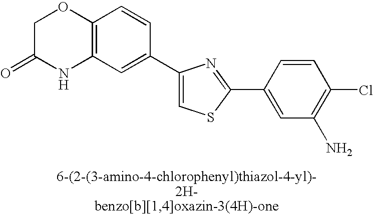 | ¹H NMR (DMSO-d6,400 MHz): 10.82 (s, 1 H),7.96 (s, 1 H), 7.58 (d, J =2.0Hz, 1 H), 7.55 (dd, J = 8.4,2.0 Hz, 1 H), 7.45 (d, J = 2.0Hz, 1 H), 7.34 (d, J = 8.4 Hz,1 H), 7.16 (dd, J = 8.4, 2.0Hz, 1 H), 7.04 (d, J = 8.4 Hz,1 H), 5.70 (s, 1 H), 4.62 (s,2 H). MS (ES⁺) 357, m/z(M + 1) 358, C₁₇H₁₂ClN₃O₂Srequires 357 |
| 291 | 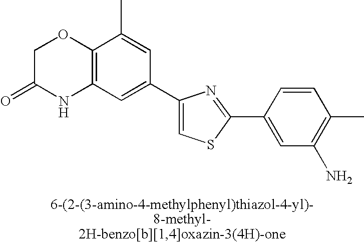 | ¹H NMR (DMSO-d6,400 MHz): 10.76 (s, 1 H),7.87 (s, 1 H), 7.46 (d, J = 1.6Hz, 1 H), 7.44 (s, 1 H), 7.41(d, J = 1.6 Hz, 1 H), 7.29 (d,J =8.0 Hz, 1 H), 7.16 (d, J =8.0 Hz, 1 H), 4.63 (s, 2 H),2.24 (s, 3 H), 2.17 (s, 3 H).MS (ES⁺) 351, m/z (M + 1)352, C₁₉H₁₇N₃O₂S requires351 |
| 292 | 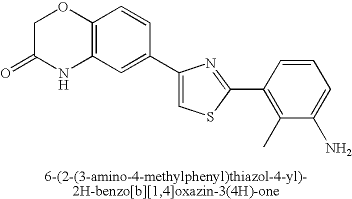 | ¹H NMR (DMSO-d6,400 MHz): 10.83 (s, 1 H),8.02 (s, 1 H), 7.59 (d, J = 2.0Hz, 1 H), 7.54 (dd, J = 8.0,2.0 Hz, 1 H), 7.13 (d, J = 8.0Hz, 1 H), 7.06 (d, J = 8.0 Hz,1 H), 7.03 (d, J = 8.0 Hz,1 H), 6.97 (d, J = 8.0 Hz,1 H), 4.62 (s, 2 H), 2.36 (s,3 H). MS (ES⁺) 337, m/z(M + 1) 338, C₁₈H₁₅N₃O₂Srequires 337 |
| 293 | 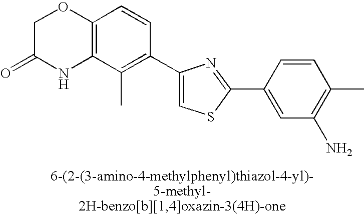 | ¹H NMR (DMSO-d6,400 MHz): 10.33 (s, 1 H),7.63 (s, 1 H), 7.22 (d, J = 8.0Hz, 1 H), 7.16 (d, J = 8.4 Hz,1 H), 7.12 (d, J = 8.0 Hz,1 H), 6.92 (d, J = 8.4 Hz,1 H), 4.57 (s, 2 H), 2.31 (s,3 H), 2.15 (s, 3 H). MS (ES⁺)351, m/z (M + 1) 352,C₁₉H₁₇N₃O₂S requires 351 |
| 294 | 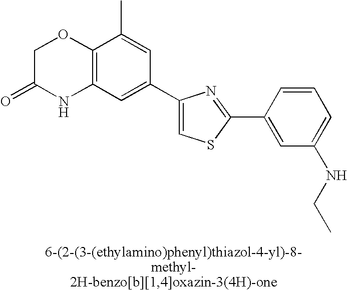 | ¹H NMR (DMSO-d6,400 MHz): 10.76 (s, 1 H),7.89 (s, 1 H), 7.46 (d, J = 1.6Hz, 1 H), 7.44 (d, J = 1.6 Hz,1 H), 7.23 (t, J = 8.0 Hz,1 H), 7.20 (d, J = 8.0 Hz,1 H), 7.17 (d, J = 8.0 Hz,1 H), 6.70 (m, 1 H), 4.63 (s,2 H), 3.10 (q, J = 0.8 Hz,2 H), 2.24 (s, 3 H), 1.20 (t, J =0.8 Hz, 3 H). MS (ES⁺)365, m/z (M + 1) 366,C₂₀H₁₉N₃O₂S requires 365 |
| 295 | 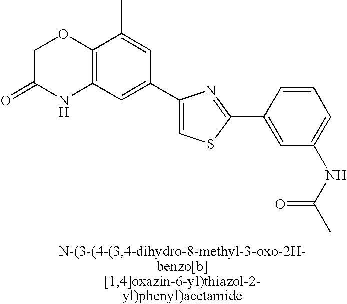 | ¹H NMR (DMSO-d6,400 MHz): 10.78 (s, 1 H),10.19 (s, 1 H), 8.29 (s, 1 H),7.95 (s, 1 H), 7.70 (d, J = 0.8Hz, 1 H), 7.66 (d, J = 0.8 Hz,1 H), 7.46 (m, 3 H), 4.64 (s,2 H), 2.24 (s, 3 H), 2.09 (s,3 H). MS (ES⁺) 379, m/z(M + 1) 380, C₂₀H₁₇N₃O₃Srequires 379 |
| 296 | 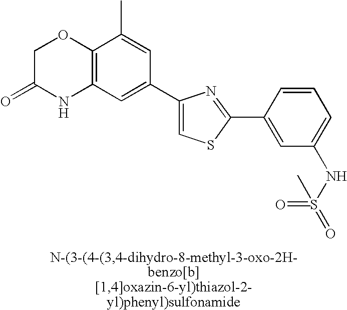 | ¹H NMR (DMSO-d6,400 MHz): 10.79 (s, 1 H),9.99 (s, 1 H), 7.98 (s, 1 H),7.86 (t, J = 2.0 Hz, 1 H),7.72 (d, J = 8.0 Hz, 1 H),7.51 (d, J = 8.0 Hz, 1 H),7.48 (m, 1 H), 7.43 (d, J =2.0 Hz, 1 H), 7.36 (m, 1 H),4.64 (s, 2 H), 3.06 (s, 3 H),2.24 (s, 3 H). MS (ES⁺) 415,m/z (M + 1) 416,C₁₉H₁₇N₃O₄S requires 415 |
| 297 | 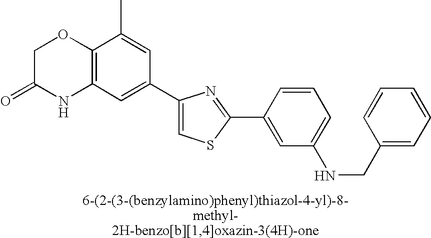 | ¹H NMR (DMSO-d6,400 MHz): 10.76 (s, 1 H),7.87 (s, 1 H), 7.46 (s, 1 H),7.41 (m, 3 H), 7.34 (m, 2 H),7.22 (m, 2 H), 7.17 (m, 2 H),6.69 (m, 1 H), 4.56 (s, 2 H),4.35 (s, 2 H), 2.24 (s, 3 H).MS (ES⁺) 427, m/z (M + 1)428, C₂₅H₂₁N₃O₂S requires427 |
| 298 | 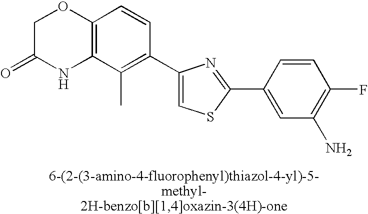 | ¹H NMR (DMSO-d6,400 MHz): 10.32 (s, 1 H),7.64 (s, 1 H), 7.44 (d, J = 8.4Hz, 1 H), 7.15 (d, J = 8.4 Hz,1 H), 7.11 (d, J = 8.4 Hz,2 H), 6.91 (d, J = 8.4 Hz,1 H), 4.52 (s, 2 H), 2.31 (s,3 H). MS (ES⁺) 355, m/z(M + 1) 356, C₁₈H₁₄FN₃O₂Srequires 355 |
| 299 | 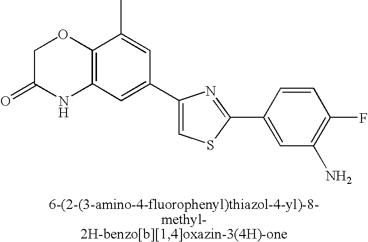 | ¹H NMR (DMSO-d6,400 MHz): 10.75 (s, 1 H),7.87 (s, 1 H), 7.46 (d, J = 1.6Hz, 1 H), 7.42 (m, 1 H), 7.41(d, J = 1.6 Hz, 1 H), 7.13 (m,2 H), 4.63 (s, 2 H), 2.24 (s,3 H). MS (ES⁺) 355, m/z(M + 1) 356, C₁₈H₁₄FN₃O₂Srequires 355 |
| 300 | 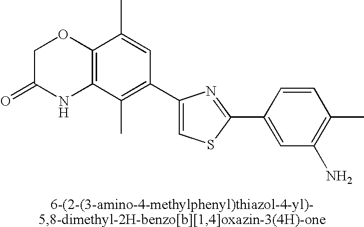 | ¹H NMR (DMSO-d6,400 MHz): 10.25 (s, 1 H),7.59 (s, 1 H), 7.34 (s, 1 H),7.13 (d, J = 8.0 Hz, 1 H),7.09 (d, J = 8.0 Hz, 1 H),7.06 (s, 1 H), 4.57 (s, 2 H),2.28 (s, 3 H), 2.20 (s, 3 H),2.13 (s, 3 H). MS (ES⁺) 365,m/z (M + 1) 366,C₂₀H₁₉N₃O₂S requires 365 |
| 301 | 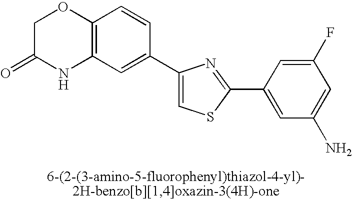 | ¹H NMR (DMSO-d6,400 MHz): 10.80 (s, 1 H),7.98 (s, 1 H), 7.59 (d, J = 2.0Hz, 1 H), 7.55 (dd, J = 8.4,2.0 Hz, 1 H), 7.04 (m, 2 H),6.88 (m, 1 H), 6.45 (m, 1 H),4.62 (s, 2 H). MS (ES⁺) 341,m/z (M + 1) 342,C₁₇H₁₂FN₃O₂S requires 341 |
| 302 | 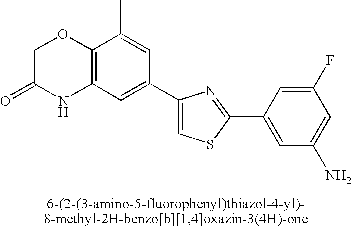 | ¹H NMR (DMSO-d6,400 MHz): 10.74 (s, 1 H),7.93 (s, 1 H), 7.46 (d, J = 1.6Hz, 1 H), 7.43 (d, J = 1.6 Hz,1 H), 7.04 (s, 1 H), 6.88 (m,1 H), 6.45 (dt, J = 11.6, 2.0Hz, 1 H), 4.63 (s, 2 H), 2.24(s, 3 H). MS (ES⁺) 355, m/z(M + 1) 356, C₁₈H₁₄FN₃O₂Srequires 355 |
| 303 | 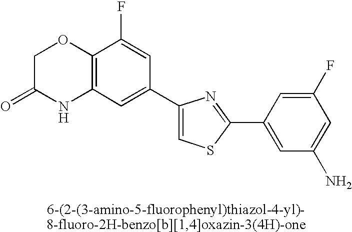 | ¹H NMR (DMSO-d6,400 MHz): 10.99 (s, 1 H),8.08 (s, 1 H), 7.54 (dd, J =12, 1.6 Hz, 1 H), 7.45 (s,1 H), 7.03 (t, J = 1.6 Hz, 1 H),6.88 (m, 1 H), 6.45 (m, 1 H),4.72 (s, 2 H). MS (ES⁺) 359,m/z (M + 1) 360,C₁₇H₁₁F₂N₃O₂S requires 359 |
| 304 | 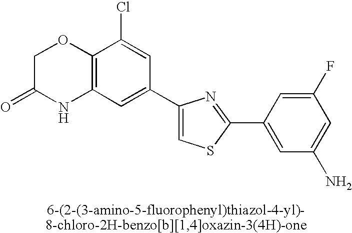 | ¹H NMR (DMSO-d6,400 MHz): 10.99 (s, 1 H),8.11 (s, 1 H), 7.72 (d, J = 2.0Hz, 1 H), 7.55 (d, J = 2.0 Hz,1 H), 7.04 (s, 1 H), 6.88 (m,1 H), 6.45 (m, 1 H), 4.76 (s,2 H). MS (ES⁺) 375, m/z(M + 1) 376,C₁₇H₁₁ClFN₃O₂S requires375 |
| 305 | 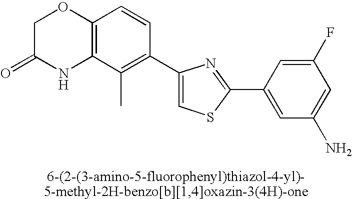 | ¹H NMR (DMSO-d6,400 MHz): 10.33 (s, 1 H),7.70 (s, 1 H), 7.16 (d, J = 8.4Hz, 1 H), 7.05 (t, J = 1.6 Hz,1 H), 6.92 (d, J = 8.4 Hz,1 H), 6.83 (d, J = 9.6 Hz,1 H), 6.43 (d, J = 11.6 Hz,1 H), 4.57 (s, 2 H), 2.30 (s,3 H). MS (ES⁺) 355, m/z(M + 1) 356, C₁₈H₁₄FN₃O₂Srequires 355 |
| 306 | 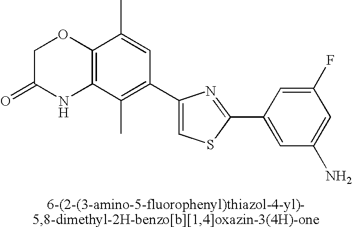 | ¹H NMR (DMSO-d6,400 MHz): 10.25 (s, 1 H),7.68 (s, 1 H), 7.06 (m, 2 H),6.83 (dt, J = 9.6, 1.6 Hz,1 H), 6.43 (dt, J = 11.6, 1.6Hz, 1 H), 4.57 (s, 2 H), 2.27(s, 3 H), 2.19 (s, 3 H). MS(ES⁺) 369, m/z (M + 1) 370,C₁₉H₁₆FN₃O₂S requires 369 |

Compounds from table 5 were prepared according to reference 7.

TABLE 5

| Compound | | Physical Data ¹H NMR 400 MHz |
|---|---|---|
| Number | Structure | (CDCl₃ or DMSO) and/or MS (m/z) (M + 1)⁺ |
| 307 | 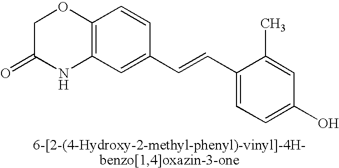 | ¹H NMR (400 MHz, DMSO-_{d6}) δ 10.70 (s, 1H),9.43 (s, 1H), 7.48 (d, J = 8.8 Hz, 1H), 7.05-7.16(m, 3H), 6.93 (d, J = 8 Hz, 1H), 6.85 (d, J = 16Hz, 1H), 6.60-6.63 (m, 2H), 4.58 (s, 2H), 2.30 (s,3H), MS: (ES⁺) 282 m/z (M + 1)⁺ C₁₇H₁₅NO₃ requires 282 |
| 308 | 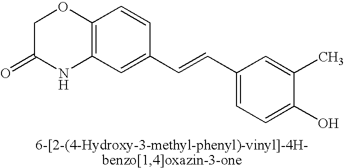 | ¹H NMR (400 MHz, DMSO-_{d6}) δ 10.83 (s, 1H),9.54 (s, 1H), 7.40 (s, 1H), 7.28 (dd, J = 8.8 Hz,3.4 Hz, 1H), 7.19 (dd, J = 8.7 Hz, 2.2 Hz, 1H),7.09 (d, J = 1.9 Hz, 1H), 7.03-6.93 (m, 3H), 6.84(d, J = 8.2 Hz, 1H), 4.65 (s, 2H), 2.22 (s, 3H).MS: (ES⁺) 282 m/z (M + 1)⁺ C₁₇H₁₅NO₃ requires282 |
| 309 | 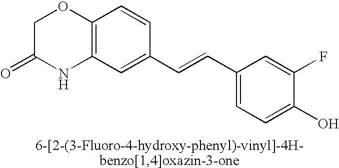 | ¹H NMR (400 MHz, DMSO-_{d6}) δ 10.77 (s, 1H),9.97 (s, 1H), 7.44 (dd, J = 12.6 Hz, 2.9 Hz, 1H),7.19 (m, 1H), 7.13 (m, 1H), 7.05-7.00 (m, 2H),6.96-6.88 (m, 3H), 4.58 (s, 2H). MS: (ES⁺) 286m/z (M + 1)⁺ C₁₆H₁₂FNO₃ requires 286 |
| 310 | 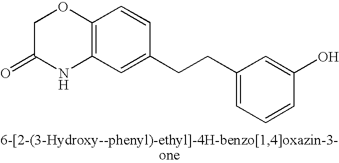 | MS: (ES⁺) 270 m/z (M + 1)⁺ C₁₆H₁₅NO₃ requires270 |
| 311 | 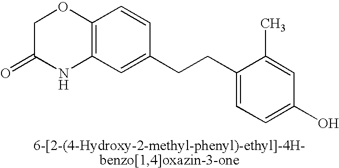 | MS: (ES⁺) 284 m/z (M + 1)⁺ C₁₇H₁₇NO₃ requires284 |
| 312 | 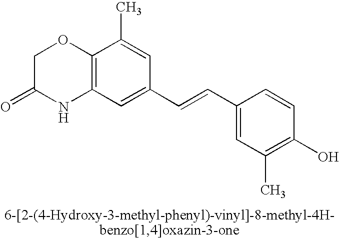 | ¹H NMR (400 MHz, DMSO-_{d6}) δ 10.70 (s, 1H),9.40 (s, 1H), 7.30 (s, 1H), 7.18 (dd, J = 8.0 Hz,1.6 Hz, 1H), 7.02 (d, J = 1.6 Hz, 1H), 6.88 (s,2H), 6.84 (s, 1H), 6.74 (d, J = 8.4 Hz, 1H), 4.58(s, 2H), 2.18 (s, 3H), 2.14 (s, 3H). MS: (ES⁺) 296m/z (M + 1)⁺ C₁₈H₁₇NO₃ requires 296 |

Compounds from table 6 were prepared according to reference 8.

TABLE 6

| Compound | | Physical Data ¹H NMR 400 MHz |
|---|---|---|
| Number | Structure | (CDCl₃ or DMSO) and/or MS (m/z) (M + 1)⁺ |
| 313 | 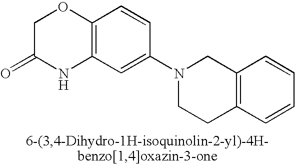 | ). ¹H NMR (400 MHz, CD₃OD,) δ 7.04-7.06 (m, 4H),6.77 (d, J = 8.8 Hz, 1H), 6.59 (dd, J = 2.8, 8.8 Hz,1H), 6.52 (d, J = 2.8 Hz, 1H), 4.40 (s, 2H), 4.18 (s,2H), 3.36 (d, J = 6 Hz, 2H), 2.87 (d, J = 6 Hz, 2H).MS: (ES⁺) 281 m/z (M + 1)⁺ C₁₇H₁₇N₂O₂ requires 281 |
| 314 | 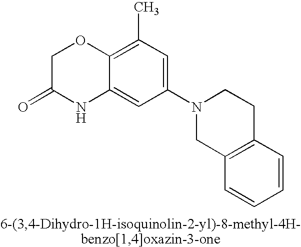 | ¹H NMR (400 MHz, DMSO-_{d6}) δ 10.50 (s, 1H),7.20-7.13 (m, 4H), 6.52 (d, J = 2.4 Hz, 1H), 6.39 (d,J = 2.4 Hz, 1H), 4.47 (s, 2H), 4.24 (s, 2H), 3.38 (t,J = 6.0 Hz, 2H), 2.89 (t, J = 6.0 Hz, 2H), 2.17 (s, 3H).MS: (ES⁺) 294 m/z (M + 1)⁺ C₁₈H₁₈N₂O₂ requires 294 |
| 315 | 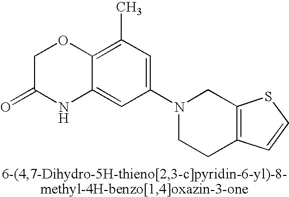 | ¹H NMR (400 MHz, DMSO-_{d6}) δ 10.45 (s, 1H), 7.27(d, J = 5.2 Hz, 1H), 6.82 (d, J = 5.2 Hz, 1H), 6.52 (s,1H), 6.39 (s, 1H), 4.41 (s, 2H), 4.12 (s, 2H), 3.42 (s,2H), 2.85 (s, 2H), 2.06 (s, 3H). MS: (ES⁺) 301 m/z(M + 1)⁺ C₁₆H₁₇N₂O₂S requires 301 |
| 316 | 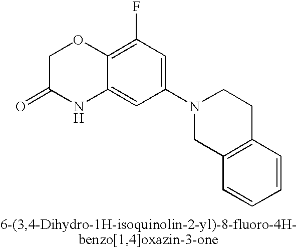 | ¹H NMR (400 MHz, DMSO-_{d6}) δ, 10.70 (s, 1H),7.20-7.14 (m, 4H), 6.56 (dd, J = 14.0 Hz, 2.8 Hz, 1H),6.34-6.32 (m, 1H), 4.54 (s, 2H), 4.28 (s, 2H), 3.42 (t,J = 6.0 Hz, 2H), 2.88 (t, J = 6.0 Hz, 2H). MS: (ES⁺)299 m/z (M + 1)⁺ C₁₇H₁₅FN₂O₂ requires 299 |
| 317 | 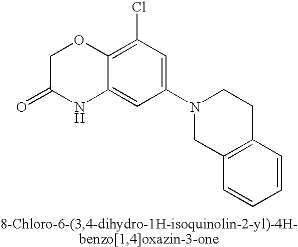 | ¹H NMR (400 MHz, DMSO-_{d6}) δ, 10.70 (s, 1H),7.20-7.14 (m, 4H), 6.68 (d, J = 2.6 Hz, 1H), 6.48 (d,J = 2.6 Hz, 1H), 4.57 (s, 2H), 4.28 (s, 2H), 3.42 (t,J = 6.0 Hz, 2H), 2.88 (t, J = 6.0 Hz, 2H). MS: (ES⁺)315 m/z (M + 1)⁺ C₁₇H₁₅ClN₂O₂ requires 315 |
| 318 | 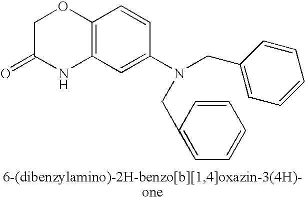 | ¹H NMR (400 MHz, CDCl₃) δ, 7.33 (broad s, 1H),7.21 (m, 5H), 7.11 (m, 5H), 6.66 (d, J = 8.8 Hz, 1H),6.21 (dd, J = 7.6, 2.4 Hz, 1H), 5.97 (s, 1H), 4.47 (s,4H), 4.37 (s, 2H). MS: (ES⁺) 344 m/z (M + 1)⁺C₂₂H₂₀FN₂O₂ requires 344 |

### Example 319

### 6-(2-Phenyl-cyclopropyl)-4H-benzo[1,4]oxazin-3-one

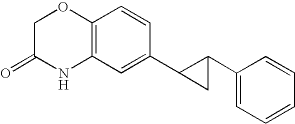

To a 40 mL scintillation vial is charged 3-oxo-6-styryl-2,3-dihydro-benzo[1,4]oxazine-4-carboxylic acid tert-butyl ester (85 mg, 0.241 mmol), 1,2-dichloroethane (5 mL), diethyl zinc (0.725 mL of 1 M hexanes solution, 0.725 mmol) and cooled to 0° C. Via syringe, chloro-iodo-methane (88 μL, 1.2 mmol) is added over 5 min. Upon completion of the addition the cooling bath is removed and the reaction is heated to 50° C. for 1 h. After 1 h at the reaction is cooled to 0° C. diluted with dichloromethane (5 mL), and quenched with saturated ammonium chloride (5 mL). The mixture is then worked up using a standard aqueous/ethyl acetate workup. The organic layers are removed under reduced pressure to afford a clear oil. The residue is treated with 30% trifluoroacetic acid in dichloromethane (˜5 mL) and the t-boc group is removed within 20 min. The solvent is removed and the product is purified from the reaction mixture by preparative LCMS. ¹H NMR (400 MHz, DMSO-d₆) δ 10.66 (s, 1H), 7.26-7.30 (m, 2H), 7.15-7.18 (m, 3H), 6.68 (d, J=8.4 Hz, 1H), 6.73 (dd, J=2, 8.4 Hz, 1H), 6.67-6.68 (m. 1H), 4.52 (s, 2H), 2.03-2.15 (m, 2H), 1.32-1.44 (m, 2H). MS: (ES⁺) 266 m/z (M+1)⁺ C₁₇H₁₆NO₂ requires 266.

### Example 320

### 3-Oxo-6-(2-pyridin-3-yl-thiazol-4-yl)-3,4-dihydro-2H-benzo[1,4]oxazine-8-carbonitrile

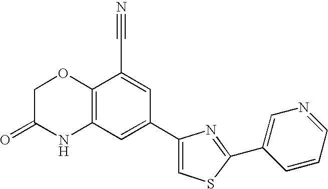

Example 320 is prepared via heating 8-chloro-6-(2-pyridin-3-yl-thiazol-4-yl)-4H-benzo[1,4]oxazin-3-one (0.5 mmol, 1 eq), ZnCN₂ (2 eq), Pd(PPH₃)₄ (0.1 eq) in DMA under and argon atmosphere at 150° C. for 30 min. The reaction mixture is filtered and the product is purified from the reaction mixture via HPLC. ¹H NMR (400 MHz, DMSO-d₆) δ 11.04 (s, 1H), 9.13 (d, J=1.6 Hz, 1H), 8.63 (dd, J=3.2 Hz, 4.8 Hz, 1H), 8.28-8.31 (m, 1H), 8.22 (s, 1H), 7.97 (d, J=2.0 Hz), 7.81 (d, J=1.6 Hz, 1H), 7.49-7.53 (m, 1H), 4.49 (s, 2H). MS: (ES⁺) m/z (M+1)⁺ C₁₇H₁₁N₄O₂S requires 335.

### Example 321

### 6-(2-Pyridin-3-yl-oxazol-5-yl)-4H-benzo[1,4]oxazin-3-one

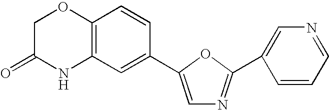

Example 321 is prepared starting with the displacement of hexamine (133 mmol, 1.5 eq) and 6-(2-chloro-acetyl)-4H-benzo[1,4]oxazin-3-one in dioxane at reflux for 18 h. The reaction was cooled and the product was filtered from the reaction mixture and used directly in the next step. The product of the first reaction was converted to the primary amine by heating in MeOH and 10% v/v conc HCl at 50° C. for 2 h and then filtering the 6-(2-amino-acetyl)-4H-benzo[1,4]oxazin-3-one hydrochloride. The reactin of 6-(2-amino-acetyl)-4H-benzo[1,4]oxazin-3-one (1 mmol, 1 eq) and nicotinoyl chloride in (1 mmol, 1 eq) in and triethylamine (10 mmol, 10 eq), THF afforded the desired N-[2-oxo-2-(3-oxo-3,4-dihydro-2H-benzo[1,4]oxazin-6-yl)-ethyl]-nicotinamide after and standard aqueous/EtOAc workup. The N-[2-oxo-2-(3-oxo-3,4-dihydro-2H-benzo[1,4]oxazin-6-yl)-ethyl]-nicotinamide was then treated with Burgess reagent (1 mmol, 1 eq) in THF at 100° C. for 10 min. The product was then purified from the reaction mixture by HPLC. ¹H NMR (400 MHz, DMSO-d₆) δ 10.78 (s, 1H), 9.14 (d, J=1.6 Hz, 1H), 8.65 (dd, J=3.2 Hz, 4.8 Hz, 1H), 8.31-8.29 (m, 1H), 7.67 (s, 1H), 7.56-7.53 (m, 1H), 7.38 (dd, J=6.4 Hz, 8.4 Hz, 1H), 7.24 (d, J=2.0 Hz, 1H), 7.01 (d, J=8.4 Hz, 1H), 4.57 (s, 2H). MS: (ES⁺) 294 m/z (M+1)⁺ C₁₆H₁₂N₃O₃ requires 294.

### Example 322

### 6-(2-Phenyl-oxazol-4-yl)-4H-benzo[1,4]oxazin-3-one

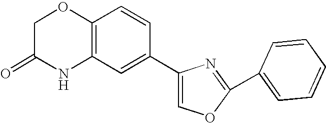

Example 322 was synthesized according to the procedure described for examples 321 from 6-(2-chloro-acetyl)-4H-benzo[1,4]oxazin-3-one (226 mg, 1 mmol) and benzamide (125 mg, 1 mmol). The reaction is heated to 250° C. for 10 min and then cooled to room temperature. The black residue is dissolved in DMSO and the product purified from the reaction mixture via preparative HPLC. ¹H NMR (400 MHz, DMSO-d₆) δ 10.93 (s, 1H), 8.73 (s, 1H), 8.13-8.15 (m, 2H), 7.67-7.68 (m, 3H), 7.50-7.54 (m, 2H), 7.14 (d, J=8 Hz, 1H), 7.11 (d, J=4 Hz, 1H), 6.85 (d, J=8 Hz, 1H), 4.75 (s, 2H). MS: (ES⁺) 293 m/z (M+1)⁺ C₁₇H₁₃N₂O₃ requires 293.

### Example 323

### 4-Methanesulfonyl-6-(2-phenyl-thiazol-4-yl)-4H-benzo[1,4]oxazin-3-one

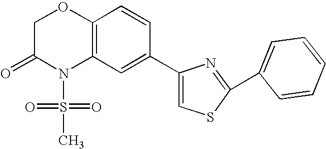

Example 323 is prepared using 6-(2-phenyl-thiazol-4-yl)-4H-benzo[1,4]oxazin-3-one and methanesulfonyl chloride. ¹H NMR (400 MHz, DMSO-d₆) δ 8.14 (3, J=2.0 Hz, 1H), 8.09 (s, 1H), 7.94 (dd, J=6.0 Hz, 8.0 Hz, 2H), 7.81 (dd, J=6.4 Hz, 8.4 Hz, 1H), 7.50-7.44 (m, 3H), 7.20 (d, J=8.4 Hz, 1H), 4.34 (s, 2H), 3.73 (s, 3H). MS: (ES⁺) 387 m/z (M+1)⁺ C₁₈H₁₅N₂O₄S₂ requires 387.

### Example 324

### 4-Acetyl-6-[4-(3-bromo-phenyl)-thiazol-2-yl]-4H-benzo[1,4]oxazin-3-one

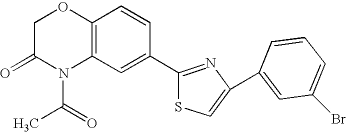

Example 324 is prepared using 6-(4-(3-bromophenyl)thiazol-2-yl)-2H-benzo[b][1,4]oxazin-3(4H)-one and acetyl chloride. ¹H NMR (400 MHz, DMSO-d₆) δ 10.75 (s, 1H), 8.24 (s, 1H), 8.22 (d, J=2.4 Hz, 1H), 8.15 (t, J=1.6 Hz, 1H), 7.97 (d, J=8.0 Hz, 1H), 7.76 (dd, J=6.4 Hz, 8.4 Hz, 1H), 7.51-7.49 (m, 1H), 7.37 (t, J=7.6 Hz, 1H), 7.20 (d J=8.4 Hz, 1H), 4.74 (s, 2H), 2.57 (s, 3H) MS: (ES⁺) 430 m/z (M+1)⁺ C₁₉H₁₄BrN₂O₃S requires 430.

### Example 325

### 8-Methyl-6-[3-(2,2,2-trifluoro-1-hydroxy-ethyl)-phenyl]-4H-benzo[1,4]oxazin-3-one

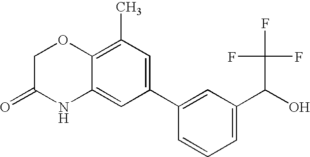

Example 325 is prepared by heating 3-(8-methyl-3-oxo-3,4-dihydro-2H-benzo[1,4]oxazin-6-yl)-benzaldehyde (0.5 mmol, 2 eq) and TMSCF₃ (1.0 mmol, 2 eq) in at 60° C. overnight under and atmosphere of argon. The reaction mixture was concentrated to dryness and the product was purified via HPLC. ¹H NMR (400 MHz, DMSO-d₆) δ 10.64 (s, 1H), 7.58 (s, 1H), 7.48-7.46 (m, 1H), 7.37 (d, J=1.6 Hz, 1H), 7.02 (d, J=1.6 Hz, 1H), 6.92 (d, J=1.6 Hz, 1H), 6.82 (d J=5.6 Hz, 1H), 5.17-5.14 (m, 1H), 4.54 (s, 2H), 2.15 (s, 3H).MS: (ES⁺) 338 m/z (M+1)⁺ C₁₇H₁₅F₃NO₃ requires 338.

### Example 326

### 6-[3-Chloro-5-(1-hydroxy-ethyl)-phenyl]-8-methyl-4H-benzo[1,4]oxazin-3-one

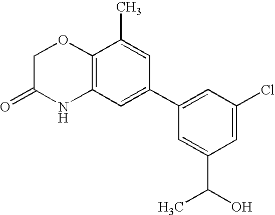

Example 326 is prepared via charging 3-chloro-5-(8-methyl-3-oxo-3,4-dihydro-2H-benzo[1,4]oxazin-6-yl)-benzaldehyde (0.2 mmol, 1 eq) to a vial and diluting with THF (3 mL) under and atmosphere of argon. The reaction vial was cooled to 0° C. and then MeMgBr (0.2 mmol, 1 eq) was added. Upon completion of the addition the reaction was quenched with saturated ammonium chloride, the organic layers were separated, dried with MgSO₄ and concentrated. The product was then purified from the reaction mixture by HPLC. ¹H NMR (400 MHz, DMSO-d₆) δ 10.59 (s, 1H), 7.58 (d, J=8.0 Hz, 1H), 7.45 (d, J=2.0 Hz, 1H), 7.43 (s, 1H), 7.07 (J=4.6 Hz, 1H), 6.90, (d, J=2.0 Hz, 1H), 5.29 (d, J=4.4 Hz, 1H), 4.96-4.94 (m, 1H), 4.53 (s, 1H), 2.13 (s, 3H), 1.24 (d, J=6.4 Hz, 3H), 1.06 (s, 2H). MS: (ES⁺) 319 m/z (M+1)⁺ C₁₇H₁₇ClNO₃ requires 319.

### Example 327

### 8-Methyl-6-(3-pyrazol-1-ylmethyl-phenyl)-4H-benzo[1,4]oxazin-3-one

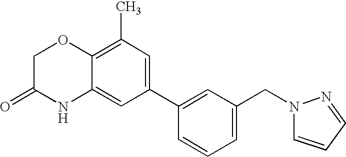

Example 327 is prepared by reacting methanesulfonic acid 3-(8-methyl-3-oxo-3,4-dihydro-2H-benzo[1,4]oxazin-6-yl)-benzyl ester (0.1 mmol, 1 eq) and pyrazole (0.3 mmol, 3 eq) in DMF (1 mL) at 50° C. overnight and then purfication of the product via HPLC. ¹H NMR (400 MHz, DMSO-d₆) δ 10.62 (s, 1H), 7.77 (d, J=2.0 Hz, 1H), 7.38 (d, J=1.2 Hz, 1H), 7.39-7.29 (m, 4H), 7.06 (d, J=7.2 Hz, 1H), 6.98 (d, J=4.6 Hz, 1H), 6.86 (d, J=2.4 Hz, 1H), 6.19 (t, J=2.0 Hz, 1H), 5.30 (s, 2H), 4.53 (s, 2H), 2.13 (s, 3H). MS: (ES⁺) 320 m/z (M+1)⁺ C₁₉H₁₈N₃O₂ requires 320.

### Example 328

### 6-[3-(3-Trifluoromethyl-phenyl)-acryloyl]-4H-benzo[1,4]oxazin-3-one

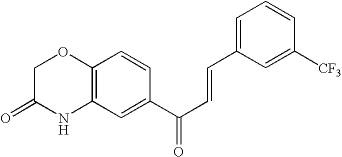

Example 328 is prepared via heating 6-acetyl-4H-benzo[1,4]oxazin-3-one (1 mmol, 1 eq), 3-trifluoromethyl-benzaldehyde (1 mmol, 1 eq) and Ba(OH)₂ (2 mmol, 2 eq) in EtOH at reflux for 18 h. The product was then purified from the reaction mixture via HPLC. ¹H NMR (400 MHz, DMSO-d₆) δ 10.91 (s, 1H), 8.10 (d, J=8.4 Hz, 2H), 8.02 (d, J=15.6 Hz, 1H), 7.95 (dd, J=6.4 Hz, 8.4 Hz, 1H), 7.82 (d, J=8.4 Hz, 2H), 7.77 (d, J=15.6 Hz, 1H), 7.62 (d, J=2.0 Hz, 1H), 7.12 (d, J=8.4 Hz, 1H), 4.72 (s, 2H), MS: (ES⁺) 348 m/z (M+1)⁺ C₁₈H₁₃F₃NO₃ requires 348.

### Example 329

### 4-[3-(3-Oxo-3,4-dihydro-2H-benzo[1,4]oxazin-6-yl)-5-phenyl-4,5-dihydro-pyrazol-1-yl]-benzonitrile

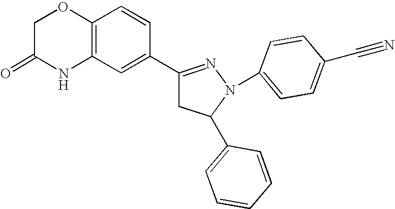

Example 329 is prepared via the condenstation of 4-cyano phenyl hydrazine and 6-(3-phenyl-acryloyl)-4H-benzo[1,4]oxazin-3-one in DMF at 180° C. for 10 min. The product was then purified from the reaction mixture via HPLC. ¹H NMR (400 MHz, DMSO-d₆) δ 10.71 (s, 1H), 7.48 (d, J=8.8 Hz, 2H), 7.35 (d, J=1.6 Hz, 1H), 7.26 (t, J=7.2 Hz, 2H), 7.15-7.20 (m, 4H), 6.92 (t, J=9.2 Hz, 3H), 5.54 (dd, J=6.8 Hz, 4.6 Hz, 1H), 4.55 (s, 2H), 3.27 (s, 2H). MS: (ES⁺) 395 m/z (M+1)⁺ C₂₄H₁₉N₄O₂ requires 395.

### Example 330

### 6-(1-Phenyl-1H-pyrazol-3-yl)-4H-benzo[1,4]oxazin-3-one

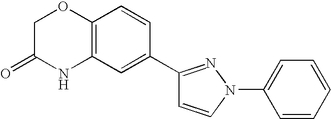

Example 330 was prepared via the condensation of 6-acetyl-4H-benzo[1,4]oxazin-3-one with dimethyl formamide dimethyl acetal at 150° C. for 10 min. The resultant 6-(3-dimethylamino-acryloyl)-4H-benzo[1,4]oxazin-3-one was then reacted with phenylhydrazine at 150° C. for 10 min. The product was then purified from the reaction mixture via HPLC ¹H NMR (400 MHz, DMSO-d₆) δ 10.65 (s, 1H), 7.65 (d, J=1.6 Hz, 1H), 7.36-7.26 (m, 3H), 7.20-7.18 (m, 2H), 6.82 (d, J=8.4 Hz, 1H), 6.72 (d, J=2.0 Hz, 1H), 6.63 (dd, J=6.0 Hz, 8.0 Hz, 1H), 6.47 (d, J=2.0 Hz, 1H), 4.51 (s, 2H). MS: (ES⁺) 292 m/z (M+1)⁺ C₁₇H₁₄N₃O₂ requires 292.

### Example 331

### 6-(1,5-Diphenyl-1H-pyrazol-3-yl)-4H-benzo[1,4]oxazin-3-one

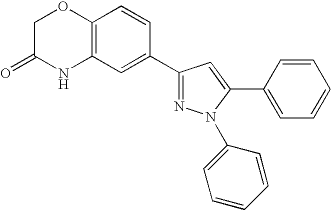

Example 331 is prepared via the condenstation of phenyl hydrazine and 6-(3-phenyl-acryloyl)-4H-benzo[1,4]oxazin-3-one in DMF at 180° C. for 10 min. The product was then purified from the reaction mixture via HPLC. ¹H NMR (400 MHz, DMSO-d₆) δ 10.80 (s, 1H), 7.93-7.89 (m, 2H), 7.50-7.35 (m, 7H), 7.08 (s, 1H), 6.94 (d, J=8.0 Hz, 1H), 6.86 (d, J=1.6 Hz, 1H) 6.82-6.77 (m, 1H), 6.55 (s, 1H), 4.62 (s, 2H). MS: (ES⁺) 368 m/z (M+1)⁺ C₂₃H₁₇N₃O₂ requires 368.

### Example 332

### 6-(3-phenyl-1,2,4-oxadiazol-5-yl)-2H-benzo[b][1,4]oxazin-3(4H)-one

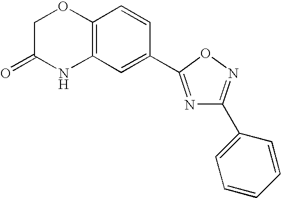

A slurry of 6-carboxy-4H-benzo[1,4]oxazin-3-one and CDI (1.1 equivalent/substrate) in DMF was stirred at RT for 30 minutes. N′-hydroxybenzenecarboximidamide (1.1 equivalent substrate) was added and the mixture was stirred overnight at 115° C. After cooling at RT and filtration over a short celite pad, the product was then purified from the reaction mixture via LC-MS. ¹H NMR (DMSO-d₆, 400 MHz): 11.00 (s, 1H), 8.08 (dd, J=0.8, 0.0 Hz, 2H), 7.77 (dd, J=0.8, 0.0 Hz, 1H), 7.72 (d, J=0.0 Hz, 1H), 7.61 (m, 3H), 7.20 (d, J=0.8 Hz, 1H), 4.75 (s, 2H). MS (ES⁺) 293, m/z (M+1) 294, C₁₆H₁₁N₃O₃ requires 293.

### Example 333

### 6-(2-Phenyl-oxazol-4-yl)-4H-benzo[1,4]oxazin-3-one

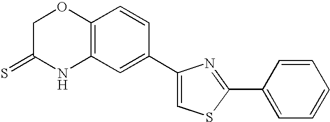

To a 40 mL vial are charged 6-(2-phenyl-thiazol-4-yl)-4H-benzo[1,4]oxazin-3-one (154 mg. 0.5 mmol), Lawesson's reagent (404 mg, 1 mmol) and tetrahydrofuran (3 mL). The reaction is heated to 80° C. for 20 min and then cooled to room temperature. The solvent is removed under reduced pressure the yellow residue is dissolved in DMSO and the product purified from the reaction mixture via preparative HPLC. ¹H NMR (400 MHz, DMSO-d₆) δ 12.85 (s, 1H), 8.01-8.04 (m, 3H), 7.84 (s, 1H), 7.70 (d, J=8 Hz, 1H), 7.54-7.56 (m, 4H), 7.01 (d, J=8 Hz, 1H), 4.90 (s, 2H). MS: (ES⁺) 325 m/z (M+1)⁺ C₁₇H₁₃N₂OS₂ requires 325.

### Example 334

### Functional Assay of Mineralocorticoid Receptor Antagonism

The MR antagonist activity of the compounds is determined in a mammalian two hybrid reporter system. The N-terminus of MR (MR-NT, sequence coding amino acid 1-597) is fused to the activation domain of the VP16 gene. The ligand binding domain of MR (MR-LBD, sequence encoding amino acid 672-984) is fused to the DNA binding domain of the yeast Gal4 gene. The MR gene is cloned from a human kidney cDNA library with PCR.

The assay is performed in 384 well plates. Briefly, 293T cells (ATCC) are transfected with expression vectors for Gal-4-MR-LBD and VP16-MR NT, and a luciferase reporter vector containing Gal4 binding sequence (pG5-Luc). Cells are plated in 384 well plates immediately after transfection (approximately 3×10⁴ cells/well in 50 μl medium). The medium is supplemented with 3% charcoal-dextran treated fetal bovine serum (Hyclone). Twenty four hours after transfection, compounds prepared in DMSO are transferred to the cells. The cells are then stimulated with 0.4 nM final concentration of aldosterone (Acros) and incubated at 37° C. for another 24 hours before the luciferase activity is assayed with 20 μl of Bright-Glo (Promega) using a luminometer (CLIPR). The expression of luciferase is used as an indicator of aldosterone-induced MR trans-activation. Each compound is tested in duplicates with 12-concentration titration. IC50 values (defined as the concentration of test compound required to antagonize 50% of aldosterone-induced MR activity) are determined from the dose-response curve.

### Example 335

### Functional Assay of Glucocorticoid Receptor Antagonism

The GR antagonist activity of the compounds is determined in a mammalian two hybrid reporter system. The ligand binding domain of GR (GR-LBD, sequence encoding amino acid 541-778) is fused to the DNA binding domain of the yeast Gal4 gene. The GR gene is cloned from a human lung cDNA library with PCR.

The assay is performed in 384 well plates: COS-7 cells (ATCC) are transfected with expression vectors for Gal-4-GR-LBD and a luciferase reporter vector containing Gal4 binding sequence (pG5-Luc). Cells are plated in 384 well plates immediately after transfection (approximately 8000 cells/well in 50 μl medium). The medium is supplemented with 3% charcoal-dextran treated fetal bovine serum (Hyclone). Twenty four hours after transfection, compounds prepared in DMSO are transferred to the cells. The cells are then stimulated with 10 nM final concentration of dexamethasone (Sigma) and incubated at 37° C. for another 24 hours before the luciferase activity is assayed with 20 μl of Bright-Glo (Promega) using a luminometer (CLIPR). The expression of luciferase is used as an indicator of dexamethasone-induced GR trans-activation. Each compound is tested in duplicates with a 12-concentration titration. IC50 values (defined as the concentration of test compound required to antagonize 50% of dexamethasone-induced GR activity) are determined from the dose-response curve.

### Example 336

### Functional Assay of Progesterone Receptor Antagonism

The PR antagonist activity of the compounds is determined by progesterone-induced alkaline phosphatase activity in the T-47D cell line (ATCC). In the T-47D breast cancer cells, progesterone specifically induces de novo synthesis of a membrane-associated alkaline phosphatase enzyme in a time and dose-dependent manner (Di Lorenzo et al., Cancer Research, 51: 4470-4475 (1991)). The alkaline phosphatase enzymatic activity can be measured with a chemiluminescent substrate, such as CSPD® (Applied Biosystems).

The assay is performed in 384 well plates. Briefly, T-47D cells are plated in 384 well plates at a density of approximately 2.5×10⁴ cells/well in 50 μl medium supplemented with 10% fetal bovine serum. Twenty four hours later, the medium is aspirated. New medium that is free of phenol red and serum is added to the cells. Compounds prepared in DMSO are transferred to the cells. The cells are then stimulated with 3 nM final concentration of progesterone (Sigma) and incubated at 37° C. for another 24 hours before the alkaline phosphatase is assayed with 25 μl of CSPD® (Applied Biosystems) using a luminometer (CLIPR). The expression of alkaline phosphatase is used as an indicator of progesterone-induced PR trans-activation. Each compound is tested in duplicates with a 12-concentration titration. IC50 values (defined as the concentration of test compound required to antagonize 50% of progesterone-induced PR activity) are determined from the dose-response curve.

### Example 337

### Functional Assay of Androgen Receptor Antagonism

The AR antagonist activity of the compounds is determined with the MDA-Kb2 cell line (ATCC), which stably expresses the MMTV luciferase reporter. The MMTV promoter is a mouse mammary tumor virus promoter that contains androgen receptor response elements. The MDA-kb2 cells was derived from the MDA-MB-453 cells, which has been shown to express high levels of functional, endogenous androgen receptor (Wilson et al., Toxicological Sciences, 66: 69-81 (2002)). Upon stimulation with AR ligands, such as dihydrotestosterone, the MMTV luciferase reporter can be activated.

The assay is performed in 384 well plates. Briefly, MDA-kb2 cells are plated in 384 well plates at a density of approximately 2.4×10⁴ cells/well in 50 μl medium. The medium is supplemented with 5% charcoal-dextran treated fetal bovine serum (Hyclone). Twenty four hours later, compounds prepared in DMSO are transferred to the cells. The cells are then stimulated with 0.3 nM final concentration of dihydrotestosterone (Sigma) and incubated at 37° C. for another 24 hours before the luciferase activity is assayed with 20 μl of Bright-Glo (Promega) using a luminometer (CLIPR). The expression of luciferase is used as an indicator of dihydrotestosterone-induced AR trans-activation. Each compound is tested in duplicates with a 12-concentration titration. IC50 values (defined as the concentration of test compound required to antagonize 50% of dihydrotestosterone-induced AR activity) are determined from the dose-response curve.

Compounds of Formula I, in free form or in pharmaceutically acceptable salt form, exhibit valuable pharmacological properties, for example, as indicated by the in vitro tests described in this application (Examples 141-144). The compounds of the invention preferably exhibit inhibitory activity for steroid hormone nuclear receptors with an IC50 in the range of 1×10⁻⁹ to 1×10⁻⁵M, preferably less than 500 nM, more preferably less than 250 nM. For example:

(i). acetic acid 3-methyl-4-[2-(3-oxo-3,4-dihydro-2H-benzo[1,4]oxazin-6-yl)-ethyl]-phenyl ester has an IC50 of less than 2 nM for MR;

(ii). 6-(2-o-tolyl-vinyl)-4H-benzo[1,4]oxazin-3-one has an IC50 of 54 nM and 138 nM for MR and AR, respectively;

(iii). Acetic acid 3-methyl-4-[2-(8-methyl-3-oxo-3,4-dihydro-2H-benzo[1,4]oxazin-6-yl)-ethyl]-phenyl ester has an IC50 of 1.3 nM and 210 nM for MR and GR, respectively;

(iv). 5-Methyl-6-m-tolyl-4H-benzo[1,4]oxazin-3-one has an IC50 of 47 nM and 22 nM for MR and PR, respectively; and

(v). 5-Methyl-6-[2-(2-trifluoromethyl-phenyl)-thiazol-4-yl]-4H-benzo[1,4]oxazin-3-one has an IC50 of 162 nM, 52 nM, >20 μM and >10 μM for MR, AR, PR and GR, respectively.

The compounds of the present invention are, therefore, useful for the treatment and/or prevention of diseases in which steroidal nuclear hormone receptor activity contributes to the pathology and/or symptomology of the disease.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

We claim:

1. A compound of Formula I: 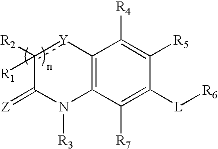
in which:
n is selected from 0, 1 and 2;
Z is selected from O and S;
Y is selected from O, S and NR₈; wherein R₈ is selected from hydrogen, C₁₋₆alkyl and halo-substituted-C₁₋₆alkyl;
L is selected from a bond, C₁₋₆alkylene, C₂₋₆alkenylene and C₂₋₆alkynylene;
wherein any alkylene can be cyclized and alkylene or alkenylene of L can optionally have a methylene replaced with C(O), O, S(O)₀₋₂, and NR₉; wherein R₉ is selected from hydrogen and C₁₋₆alkyl, halo-substituted-C₁₋₆alkyl, C₆₋₁₀aryl, C₅₋₁₀heteroaryl, C₃₋₁₂cycloalkyl and C₃₋₈heterocycloalkyl; and wherein any alkylene or alkenylene of L is optionally substituted by 1 to 3 radicals independently selected from —C(O)OR₉ and C₁₋₆alkyl;
R₁ and R₂ are independently selected from hydrogen, halo and C₁₋₆alkyl;
R₃ is selected from hydrogen, C₁₋₆alkyl, —C(O)R₁₅ and —S(O)₀₋₂R₁₅; wherein R₁₅ is selected from hydrogen, C₁₋₆alkyl, cyano, nitro and halo-substituted-C₁₋₆alkyl, C₆₋₁₀aryl and C₅₋₁₀heteroaryl; wherein any ary or heteroaryl of R₉ is optionally substituted with 1 to 3 halo radicals;
R₄ is selected from hydrogen, halo, cyano, R₆, C₁₋₆alkyl, C₁₋₆alkylthio, halo-substituted-C₁₋₆alkyl, halo-substituted-C₁₋₆alkoxy and halo-substituted-C₁₋₆alkylthio;
R₅ and R₇ are independently selected from hydrogen, halo, C₁₋₆alkyl, C₁₋₆alkoxy, C₁₋₆alkylthio, halo-substituted-C₁₋₆alkyl, halo-substituted-C₁₋₆alkoxy and halo-substituted-C₁₋₆alkylthio;
R₆ is selected from C₆₋₁₅aryl, C₅₋₁₂heteroaryl, C₃₋₁₂cycloalkyl and C₃₋₈heterocycloalkyl; wherein any aryl, heteroaryl, cycloalkyl or heterocycloalkyl of R₆ is optionally substituted with 1 to 3 radicals independently selected from halo, hydroxy, amino, cyano, nitro, C₁₋₆alkyl, cyano-C₁₋₆alkyl, hydroxy-C₁₋₆alkyl, C₁₋₆alkoxy, C₁₋₆alkthio, halo-substituted-C₁₋₆alkyl, halo-substituted-C₁₋₆alkoxy, 2,2,2-trifluoro-1-hydroxy-ethyl, —XNR₁₀R₁₀, —XC(O)NR₁₀R₁₀, —XNR₁₀C(O)R₁₀, —XNR₁₀C(O)OXR₁₁, —XOR₁₀, —XOC(O)R₁₀, —XC(O)R₁₀, —XC(O)OR₁₀, —XS(O)₀₋₂NR₁₀R₁₀ and —NR₁₀R₁₁ and R₁₁; wherein each X is independently selected from a bond, C₁₋₆alkylene, C₂₋₆alkenylene and C₂₋₆alkynylene; each R₁₀ is independently selected from hydrogen and C₁₋₆alkyl; and R₁₁ is selected from C₆₋₁₀aryl, C₆₋₁₀aryl-C₁₋₄alkoxy, C₅₋₁₀heteroaryl, C₃₋₁₂cycloalkyl and C₃₋₈heterocycloalkyl; wherein any aryl, heteroaryl, cycloalkyl or heterocycloalkyl of R₁₁ is optionally substituted with 1 to 3 radicals independently selected from halo, cyano, hydroxy, —NR₁₀R₁₀, —NR₁₀C(O)R₁₀, —NR₁₀S(O)₀₋₂R₁₀, —NR₁₀-benzyl, C₁₋₆alkoxy, C₁₋₆alkyl and halo-substituted-C₁₋₆alkyl; in which R₁₀ is as described above;
with the proviso that if n is equal to zero, R₆ is not represented by Formula II: 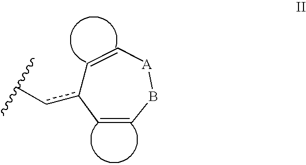
in which A and B are independently selected from O, S, C and NR₁₀; wherein R₁₀ is as described above; and the pharmaceutically acceptable salts, hydrates, solvates and isomers thereof.

2. The compound of claim 1 in which:
n is selected from 0 and 1;
Y is selected from O, S and NR₈; wherein R₈ is selected from hydrogen and C₁₋₆alkyl;
Z is selected from O and S;
L is selected from a bond, C₁₋₆alkylene, C₂₋₆alkenylene and C₂₋₆alkynylene; wherein any alkylene can be cyclized and alkylene or alkenylene of L can optionally have a methylene replaced with C(O), O, S(O)₀₋₂, and NR₉; wherein R₉ is selected from hydrogen and C₁₋₆alkyl, halo-substituted-C₁₋₆alkyl, C₆₋₁₀aryl, C₅₋₁₀heteroaryl, C₃₋₁₂cycloalkyl and C₃₋₈heterocycloalkyl; and wherein any alkylene or alkenylene of L is optionally substituted by 1 to 3 radicals independently selected from —C(O)OR₉ and C₁₋₆alkyl;
R₁ and R₂ are independently selected from hydrogen, halo and C₁₋₆alkyl;
R₃ is selected from hydrogen, C₁₋₆alkyl, —C(O)R₁₅ and —S(O)₀₋₂R₁₅; wherein R₁₅ is selected from hydrogen, C₁₋₆alkyl, cyano, nitro and halo-substituted-C₁₋₆alkyl, C₆₋₁₀aryl and C₅₋₁₀heteroaryl; wherein any ary or heteroaryl of R₉ is optionally substituted with 1 to 3 halo radicals;
R₄ is selected from hydrogen, halo, cyano, C₁₋₆alkyl and R₆;
R₅ and R₇ are independently selected from hydrogen, halo and C₁₋₆alkyl; and
R₆ is selected from C₆₋₁₅aryl, C₅₋₁₂heteroaryl, C₃₋₁₂cycloalkyl and C₃₋₈heterocycloalkyl; wherein any aryl, heteroaryl, cycloalkyl or heterocycloalkyl of R₆ is optionally substituted with 1 to 3 radicals independently selected from halo, hydroxy, amino, cyano, nitro, C₁₋₆alkyl, cyano-C₁₋₆alkyl, hydroxy-C₁₋₆alkyl, C₁₋₆alkoxy, C₁₋₆alkthio, halo-substituted-C₁₋₆alkyl, halo-substituted-C₁₋₆alkoxy, 2,2,2-trifluoro-1-hydroxy-ethyl, —XNR₁₀R₁₀, —XC(O)NR₁₀R₁₀, —XNR₁₀C(O)R₁₀, —XNR₁₀C(O)OXR₁₁, —XOR₁₀, —XOC(O)R₁₀, —XC(O)R₁₀, —XC(O)OR₁₀, —XS(O)₀₋₂NR₁₀R₁₀ and —NR₁₀R₁₁ and R₁₁; wherein each X is independently selected from a bond, C₁₋₆alkylene, C₂₋₆alkenylene and C₂₋₆alkynylene; each R₁₀ is independently selected from hydrogen and C₁₋₆alkyl; and R₁₁ is selected from C₆₋₁₀aryl, C₆₋₁₀aryl-C₁₋₄alkoxy, C₅₋₁₀heteroaryl, C₃₋₁₂cycloalkyl and C₃₋₈heterocycloalkyl; wherein any aryl, heteroaryl, cycloalkyl or heterocycloalkyl of R₁₁ is optionally substituted with 1 to 3 radicals independently selected from halo, cyano, hydroxy, —NR₁₀R₁₀, —NR₁₀C(O)R₁₀, —NR₁₀S(O)₀₋₂R₁₀, —NR₁₀-benzyl, C₁₋₆alkoxy, C₁₋₆alkyl and halo-substituted-C₁₋₆alkyl; in which R₁₀ is as described above.

3. The compound of claim 2 in which R₄ is selected from hydrogen, halo, methyl and R₆; and R₇ is selected from hydrogen and methyl.

4. The compound of claim 2 in which R₆ is selected from C₁₋₆alkyl, phenyl, thiazolyl, pyridinyl, indolyl, oxazolyl, Benzo[1,2,5]oxadiazole, 3,4-dihydro-2H-benzo[1,4]oxazine, 2,3-Dihydro-benzo[1,4]dioxine, 1H-indazolyl, 9H-thioxanthene, 6,11-dihydro-dibenzo[b,e]oxepine, 8H-indeno[1,2-d]thiazole, 5,6-dihydro-4H-cyclopentathiazole, 4,5,6,7-tetrahydro-benzothiazole, 4,5-dihydro-2-oxa-6-thia-1,3,8-triaza-as-indacene, 1,2,3,4-tetrahydro-isoquinoline, 4,5,6,7-tetrahydro-thieno[2,3-c]pyridine, naphthyl, thienyl, 1,2,3,4-tetrahydro-isoquinolinyl, 1,3-dihydro-isoindolyl, 3,4-dihydro-1H-isoquinolinyl, benzo[1,3]dioxolyl, benzo[b]furanyl, benzo[b]thienyl, benzo[1,2,5]oxadiazolyl, benzoxazolyl and 2,3-dihydro-benzo[1,4]dioxinyl; wherein R₁₀ is optionally substituted with 1 to 3 radicals independently selected from halo, methyl, trifluoromethyl, nitro, hydroxy, methyl-carbonyl-oxy, methoxy, cyano, ethyl, acetyl, methoxy-carbonyl, amino, amino-sulfonyl, methyl-carbonyl-methyl, dimethyl-amino, dimethylamino-sulfonyl, hydroxy-methyl and cyano-methyl.

5. The compound of claim 1 selected from: 6-(2-o-tolyl-vinyl)-4H-benzo[1,4]oxazin-3-one; 6-(2,2-Diphenyl-vinyl)-4H-benzo[1,4]oxazin-3-one; 6-[2-(4-Methoxy-phenyl)-vinyl]-4H-benzo[1,4]oxazin-3-one; 6-[2-(2-Ethyl-phenyl)-vinyl]-4H-benzo[1,4]oxazin-3-one; 6-[2-(2-Methylsulfanyl-phenyl)-vinyl]-4H-benzo[1,4]oxazin-3-one; 4-[2-(3-Oxo-3,4-dihydro-2H-benzo[1,4]oxazin-6-yl)-vinyl]-benzonitrile; 6-[2-(2,4-Dimethyl-phenyl)-vinyl]-4H-benzo[1,4]oxazin-3-one; 4-Methoxy-3-[2-(3-oxo-3,4-dihydro-2H-benzo[1,4]oxazin-6-yl)-vinyl]-benzonitrile; 6-[2-(6-Methoxy-naphthalen-2-yl)-vinyl]-4H-benzo[1,4]oxazin-3-one; 3-[2-(3-Oxo-3,4-dihydro-2H-benzo[1,4]oxazin-6-yl)-vinyl]-benzaldehyde; 8-Fluoro-6-(2-o-tolyl-vinyl)-4H-benzo[1,4]oxazin-3-one; 3-Methyl-4-[2-(3-oxo-3,4-dihydro-2H-benzo[1,4]oxazin-6-yl)-vinyl]-benzoic acid methyl ester; 6-(2-Pyridin-3-yl-vinyl)-4H-benzo[1,4]oxazin-3-one; 3-[2-(3-Oxo-3,4-dihydro-2H-benzo[1,4]oxazin-6-yl)-vinyl]-benzenesulfonamide; 6-[2-(3-Nitro-phenyl)-vinyl]-4H-benzo[1,4]oxazin-3-one; 6-{2-[4-(2-Oxo-propyl)-phenyl]-vinyl}-4H-benzo[1,4]oxazin-3-one; 6-(3-Phenyl-propenyl)-4H-benzo[1,4]oxazin-3-one; 6-[2-(4-Methyl-thiophen-3-yl)-vinyl]-4H-benzo[1,4]oxazin-3-one; 6-(2-Benzo[1,2,5]oxadiazol-5-yl-vinyl)-4H-benzo[1,4]oxazin-3-one; Acetic acid 3-methyl-4-[2-(8-methyl-3-oxo-3,4-dihydro-2H-benzo[1,4]oxazin-6-yl)-vinyl]-phenyl ester; 6-[2-(2-Methoxy-phenyl)-vinyl]-8-methyl-4H-benzo[1,4]oxazin-3-one; 6-[2-(4-Dimethylamino-phenyl)-vinyl]-4H-benzo[1,4]oxazin-3-one; 6-[2-(4-Hydroxy-phenyl)-vinyl]-8-methyl-4H-benzo[1,4]oxazin-3-one; 8-Methyl-6-[2-(3-nitro-phenyl)-vinyl]-4H-benzo[1,4]oxazin-3-one; 8-Methyl-6-[2-(4-methyl-thiophen-3-yl)-vinyl]-4H-benzo[1,4]oxazin-3-one; 3-(3-Oxo-3,4-dihydro-2H-benzo[1,4]oxazin-6-yl)-2-phenyl-acrylic acid methyl ester; 6-[2-(3-Nitro-phenyl)-vinyl]-4H-benzo[1,4]oxazin-3-one; 6-Styryl-4H-benzo[1,4]oxazin-3; 6-[2-(3-Trifluoromethyl-phenyl)-vinyl]-4H-benzo[1,4]oxazin-3-one; 6-(2-m-Tolyl-vinyl)-4H-benzo[1,4]oxazin-3-one; 2-[2-(3-Oxo-3,4-dihydro-2H-benzo[1,4]oxazin-6-yl)-vinyl]-4-trifluoromethyl-benzenesulfonamide; {3-[2-(3-Oxo-3,4-dihydro-2H-benzo[1,4]oxazin-6-yl)-vinyl]-phenyl}-acetonitrile; 6-[2-(2,3-Dimethyl-phenyl)-vinyl]-4H-benzo[1,4]oxazin-3-one; 6-[2-(2-Trifluoromethyl-phenyl)-vinyl]-4H-benzo[1,4]oxazin-3-one; 6-[2-(2,4-Bis-trifluoromethyl-phenyl)-vinyl]-4H-benzo[1,4]oxazin-3-one; 6-[2-(4-Trifluoromethoxy-phenyl)-vinyl]-4H-benzo[1,4]oxazin-3-one; Acetic acid 4-acetoxy-3-[2-(3-oxo-3,4-dihydro-2H-benzo[1,4]oxazin-6-yl)-vinyl]-phenyl ester; 4-[2-(3-Oxo-3,4-dihydro-2H-benzo[1,4]oxazin-6-yl)-vinyl]-3-trifluoromethyl-benzenesulfonamide; 4-[2-(3-Oxo-3,4-dihydro-2H-benzo[1,4]oxazin-6-yl)-vinyl]-benzoic acid methyl ester; 3-Fluoro-4-[2-(3-oxo-3,4-dihydro-2H-benzo[1,4]oxazin-6-yl)-vinyl]-benzenesulfonamide; 6-[2-(4-Acetyl-phenyl)-vinyl]-4H-benzo[1,4]oxazin-3-one; {4-[2-(3-Oxo-3,4-dihydro-2H-benzo[1,4]oxazin-6-yl)-vinyl]-phenyl}-acetonitrile; 6-[2-(8-Hydroxymethyl-naphthalen-1-yl)-vinyl]-4H-benzo[1,4]oxazin-3-one; 6-[2-(2-Fluoro-5-methyl-phenyl)-vinyl]-4H-benzo[1,4]oxazin-3-one; 6-[2-(4-Methyl-3,4-dihydro-2H-benzo[1,4]oxazin-7-yl)-vinyl]-4H-benzo[1,4]oxazin-3-one; 8-Fluoro-6-(2-m-tolyl-vinyl)-4H-benzo[1,4]oxazin-3-one; 3-Methyl-4-[2-(3-oxo-3,4-dihydro-2H-benzo[1,4]oxazin-6-yl)-vinyl]-benzamide; Acetic acid 3-[2-(3-oxo-3,4-dihydro-2H-benzo[1,4]oxazin-6-yl)-vinyl]-phenyl ester; Acetic acid 3,5-dimethyl-4-[2-(3-oxo-3,4-dihydro-2H-benzo[1,4]oxazin-6-yl)-vinyl]-phenyl ester; Acetic acid 2-fluoro-4-[2-(3-oxo-3,4-dihydro-2H-benzo[1,4]oxazin-6-yl)-vinyl]-phenyl ester; Acetic acid 5-[2-(3-oxo-3,4-dihydro-2H-benzo[1,4]oxazin-6-yl)-vinyl]-1H-indol-3-yl ester; 6-[2-(4-Hydroxy-2,6-dimethyl-phenyl)-vinyl]-4H-benzo[1,4]oxazin-3-one; N-{3-Methyl-4-[2-(3-oxo-3,4-dihydro-2H-benzo[1,4]oxazin-6-yl)-vinyl]-phenyl}-acetamide; 6-[2-(6-Methoxy-pyridin-2-yl)-vinyl]-4H-benzo[1,4]oxazin-3-one; 6-[2-(3-Methyl-thiophen-2-yl)-vinyl]-4H-benzo[1,4]oxazin-3-one; 4-Methyl-2-[2-(3-oxo-3,4-dihydro-2H-benzo[1,4]oxazin-6-yl)-vinyl]-benzaldehyde; 6-[2-(2,3-Dihydro-benzo[1,4]dioxin-6-yl)-vinyl]-4H-benzo[1,4]oxazin-3-one; 8-Methyl-6-[2-(6-methyl-pyridin-3-yl)-vinyl]-4H-benzo[1,4]oxazin-3-one; 3-Methyl-4-[2-(8-methyl-3-oxo-3,4-dihydro-2H-benzo[1,4]oxazin-6-yl)-vinyl]-benzoic acid methyl ester; 8-Methyl-6-[2-(4-methyl-pyridin-3-yl)-vinyl]-4H-benzo[1,4]oxazin-3-one; 6-[2-(4-Hydroxy-3-methyl-phenyl)-vinyl]-8-methyl-4H-benzo[1,4]oxazin-3-one; 6-[2-(1H-Indol-5-yl)-vinyl]-4H-benzo[1,4]oxazin-3-one; Acetic acid 4-[2-(7-fluoro-3-oxo-3,4-dihydro-2H-benzo[1,4]oxazin-6-yl)-vinyl]-phenyl; 8-Methyl-6-(2-pyridin-3-yl-vinyl)-4H-benzo[1,4]oxazin-3-one; acetic acid 4-[2-(3-oxo-3,4-dihydro-2H-benzo[1,4]oxazin-6-yl)-vinyl]-phenyl ester; 6-[2-(4-Hydroxy-2-methyl-phenyl)-vinyl]-8-methyl-4H-benzo[1,4]oxazin-3-one; 6-[2-(4-Hydroxy-phenyl)-vinyl]-4H-benzo[1,4]oxazin-3-one; 8-Fluoro-6-styryl-4H-benzo[1,4]oxazin-3-one; 6-[2-(2-Methoxy-phenyl)-vinyl]-4H-benzo[1,4]oxazin-3-one; 8-Methyl-6-[2-(3-nitro-phenyl)-vinyl]-4H-benzo[1,4]oxazin-3-one; 8-Methyl-6-styryl-4H-benzo[1,4]oxazin-3-one; 6-[2-(4-Trifluoromethyl-phenyl)-vinyl]-4H-benzo[1,4]oxazin-3-one; 6-Phenethyl-4H-benzo[1,4]oxazin-3-one; 6-(2-o-tolyl-ethyl)-4H-benzo[1,4]oxazin-3-one; 6-[2-(2-Trifluoromethyl-phenyl)-ethyl]-4H-benzo[1,4]oxazin-3-one; 6-[2-(4-Hydroxy-phenyl)-ethyl]-4H-benzo[1,4]oxazin-3-one; Acetic acid 4-[2-(8-fluoro-3-oxo-3,4-dihydro-2H-benzo[1,4]oxazin-6-yl)-ethyl]-phenyl ester; 6-(3-Phenyl-propyl)-4H-benzo[1,4]oxazin-3-one; 5-Methyl-6-phenethyl-4H-benzo[1,4]oxazin-3-one; 6-[2-(4-Methoxy-phenyl)-ethyl]-4H-benzo[1,4]oxazin-3-one; 6-(2-p-Tolyl-ethyl)-4H-benzo[1,4]oxazin-3-one; 8-Fluoro-6-[2-(2-trifluoromethyl-phenyl)-ethyl]-4H-benzo[1,4]oxazin-3-one; Acetic acid 3,5-dimethyl-4-[2-(3-oxo-3,4-dihydro-2H-benzo[1,4]oxazin-6-yl)-ethyl]-phenyl ester; Acetic acid 2-fluoro-4-[2-(3-oxo-3,4-dihydro-2H-benzo[1,4]oxazin-6-yl)-ethyl]-phenyl ester; 6-[2-(3-Fluoro-4-hydroxy-phenyl)-ethyl]-4H-benzo[1,4]oxazin-3-one; 6-(2-Benzofuran-5-yl-ethyl)-4H-benzo[1,4]oxazin-3-one; 7-Methyl-6-phenethyl-4H-benzo[1,4]oxazin-3-one; 6-[2-(4-Hydroxy-2-methyl-phenyl)-ethyl]-8-methyl-4H-benzo[1,4]oxazin-3-one; Acetic acid 3-[2-(3-oxo-3,4-dihydro-2H-benzo[1,4]oxazin-6-yl)-ethyl]-phenyl ester; Acetic acid 3-methyl-4-[2-(3-oxo-3,4-dihydro-2H-benzo[1,4]oxazin-6-yl)-ethyl]-phenyl ester; 8-Methyl-6-(2-o-tolyl-ethyl)-4H-benzo[1,4]oxazin-3-one; Acetic acid 3-methyl-4-[2-(8-methyl-3-oxo-3,4-dihydro-2H-benzo[1,4]oxazin-6-yl)-ethyl]-phenyl ester; 8-Methyl-6-phenethyl-4H-benzo[1,4]oxazin-3-one; 3,N,N-Trimethyl-4-[2-(3-oxo-3,4-dihydro-2H-benzo[1,4]oxazin-6-yl)-ethyl]-benzenesulfonamide; 6-[2-(4-Dimethylamino-phenyl)-ethyl]-4H-benzo[1,4]oxazin-3-one; 6-[2-(4-Hydroxy-phenyl)-ethyl]-8-methyl-4H-benzo[1,4]oxazin-3-one; 6-[2-(2-Methoxy-phenyl)-ethyl]-8-methyl-4H-benzo[1,4]oxazin-3-one; 8-Methyl-6-[2-(4-methyl-thiophen-3-yl)-ethyl]-4H-benzo[1,4]oxazin-3-one; 3-(3-Oxo-3,4-dihydro-2H-benzo[1,4]oxazin-6-yl)-2-phenyl-propionic acid methyl ester; {3-[2-(3-Oxo-3,4-dihydro-2H-benzo[1,4]oxazin-6-yl)-ethyl]-phenyl}-acetonitrile; 6-[2-(3,4-Dimethyl-phenyl)-ethyl]-4H-benzo[1,4]oxazin-3-one; 6-[2-(2,3-Dimethyl-phenyl)-ethyl]-4H-benzo[1,4]oxazin-3-one; 6-[2-(2,4-Dimethyl-phenyl)-ethyl]-4H-benzo[1,4]oxazin-3-one; 6-(2-Biphenyl-3-yl-ethyl)-4H-benzo[1,4]oxazin-3-one; N,N-Dimethyl-4-[2-(3-oxo-3,4-dihydro-2H-benzo[1,4]oxazin-6-yl)-ethyl]-benzenesulfonamide; 6-[2-(4-Hydroxy-3-methyl-phenyl)-ethyl]-8-methyl-4H-benzo[1,4]oxazin-3-one; Acetic acid 2-methyl-4-[2-(8-methyl-3-oxo-3,4-dihydro-2H-benzo[1,4]oxazin-6-yl)-ethyl]-phenyl ester; 6-[10,11-dihydro-dibenzo[a,d]cyclohepten-5-ylmethyl]-4H-benzo[1,4]oxazin-3-one; 6-[10,11-dihydro-dibenzo[a,d]cyclohepten-5-ylidenemethyl]-8-fluoro-4H-benzo[1,4]oxazin-3-one; 6-[10,11-dihydro-dibenzo[a,d]cyclohepten-5-ylidenemethyl]-8-methyl-4H-benzo[1,4]oxazin-3-one; 6-[10,11-dihydro-dibenzo[a,d]cyclohepten-4-hydroxy-5-ylidenemethyl]-8-methyl-4H-benzo[1,4]oxazin-3-one; 6-[10,11-dihydro-dibenzo[a,d]cyclohepten-5-ylidenemethyl]-8-trifluoromethyl-4H-benzo[1,4]oxazin-3-one; 6-[2-(4-Methoxy-phenyl)-ethyl]-4H-benzo[1,4]oxazin-3-one; 6-(2-p-Tolyl-ethyl)-4H-benzo[1,4]oxazin-3-one; 6-[2-(2-Ethyl-phenyl)-ethyl]-4H-benzo[1,4]oxazin-3-one; 8-Fluoro-6-[2-(2-trifluoro-methyl-phenyl)-ethyl]-4H-benzo[1,4]oxazin-3-one; 6-[2-(2-Methoxy-phenyl)-ethyl]-4H-benzo[1,4]oxazin-3-one; Acetic acid 3,5-dimethyl-4-[2-(3-oxo-3,4-dihydro-2H-benzo[1,4]oxazin-6-yl)-ethyl]-phenyl ester; Acetic acid 2-fluoro-4-[2-(3-oxo-3,4-dihydro-2H-benzo[1,4]oxazin-6-yl)-ethyl]-phenyl ester; Acetic acid 3-[2-(3-oxo-3,4-dihydro-2H-benzo[1,4]oxazin-6-yl)-ethyl]-phenyl ester; 6-[2-(4-Trifluoromethyl-phenyl)-ethyl]-4H-benzo[1,4]oxazin-3-one; 6-Naphthalen-2-ylmethyl-4H-benzo[1,4]oxazin-3-one; 6-Phenyl-4H-benzo[1,4]oxazin-3-one; 6-Benzofuran-2-yl-4H-benzo[1,4]oxazin-3-one; 6-Benzo[b]thiophen-3-yl-4H-benzo[1,4]oxazin-3-one; 6-Benzo[1,3]dioxol-5-yl-4H-benzo[1,4]oxazin-3-one; 6-m-Tolyl-4H-benzo[1,4]oxazin-3-one; 8-Fluoro-6-phenyl-4H-benzo[1,4]oxazin-3-one; 6-Benzofuran-5-yl-4H-benzo[1,4]oxazin-3-one; 8-Fluoro-6-m-tolyl-4H-benzo[1,4]oxazin-3-one; 8-Methyl-6-m-tolyl-4H-benzo[1,4]oxazin-3-one; 6-Benzo[1,3]dioxol-5-yl-8-methyl-4H-benzo[1,4]oxazin-3-one; 5-Methyl-6-m-tolyl-4H-benzo[1,4]oxazin-3-one; 5-m-Tolyl-3H-benzooxazol-2-one; 6-(2,3-Dihydro-benzo[1,4]dioxin-6-yl)-4H-benzo[1,4]oxazin-3-one; 3-(3-Oxo-3,4-dihydro-2H-benzo[1,4]oxazin-6-yl)-benzonitrile; 6-(5-Methyl-thiophen-2-yl)-4H-benzo[1,4]oxazin-3-one; 6-(1H-Indol-5-yl)-4H-benzo[1,4]oxazin-3-one; 6-(2,2-Difluoro-benzo[1,3]dioxol-5-yl)-8-methyl-4H-benzo[1,4]oxazin-3-one; 6-(3-Hydroxymethyl-phenyl)-8-methyl-4H-benzo[1,4]oxazin-3-one; 8-Fluoro-6-(2-methyl-1H-indol-5-yl)-4H-benzo[1,4]oxazin-3-one; 6-(3-Chloro-4-fluoro-phenyl)-8-methyl-4H-benzo[1,4]oxazin-3-one; 6-(4-Fluoro-3-methyl-phenyl)-8-methyl-4H-benzo[1,4]oxazin-3-one; 8-Fluoro-6-(1H-indol-5-yl)-4H-benzo[1,4]oxazin-3-one; 8-Chloro-6-(3-chloro-4-fluoro-phenyl)-4H-benzo[1,4]oxazin-3-one; 6-(1H-Indol-5-yl)-8-methyl-4H-benzo[1,4]oxazin-3-one; 6-(4-Hydroxymethyl-phenyl)-8-methyl-4H-benzo[1,4]oxazin-3-one; 6-Benzofuran-5-yl-8-methyl-4H-benzo[1,4]oxazin-3-one; 6-(3-Chloro-phenyl)-8-methyl-4H-benzo[1,4]oxazin-3-one; 7-Fluoro-6-m-tolyl-4H-benzo[1,4]oxazin-3-one; 6-(3-Chloro-phenyl)-7-fluoro-4H-benzo[1,4]oxazin-3-one; 7-Fluoro-6-(4-fluoro-phenyl)-4H-benzo[1,4]oxazin-3-one; 4-(7-Fluoro-3-oxo-3,4-dihydro-2H-benzo[1,4]oxazin-6-yl)-benzonitrile; [3-(7-Fluoro-3-oxo-3,4-dihydro-2H-benzo[1,4]oxazin-6-yl)-phenyl]-acetonitrile; 7-Fluoro-6-o-tolyl-4H-benzo[1,4]oxazin-3-one; 7-Fluoro-6-p-tolyl-4H-benzo[1,4]oxazin-3-one; 8-Methyl-6-(4-trifluoromethyl-phenyl)-4H-benzo[1,4]oxazin-3-one; 5-(3-Chloro-phenyl)-3H-benzooxazol-2-one; 8-Methyl-6-m-tolyl-4H-benzo[1,4]oxazin-3-one; 8-Methyl-6-thiophen-3-yl-4H-benzo[1,4]oxazin-3-one; 6-(5-Pyridin-3-yl-thiophen-2-yl)-4H-benzo[1,4]oxazin-3-one; 3-(8-Methyl-3-oxo-3,4-dihydro-2H-benzo[1,4]oxazin-6-yl)-benzonitrile; 6-(1H-Indol-5-yl)-4H-benzo[1,4]oxazin-3-one; 2-Fluoro-4-(8-methyl-3-oxo-3,4-dihydro-2H-benzo[1,4]oxazin-6-yl)-benzaldehyde; 4-(8-Methyl-3-oxo-3,4-dihydro-2H-benzo[1,4]oxazin-6-yl)-benzonitrile; 2-Methyl-4-(3-oxo-3,4-dihydro-2H-benzo[1,4]oxazin-6-yl)-benzonitrile; 2-Methyl-4-(8-methyl-3-oxo-3,4-dihydro-2H-benzo[1,4]oxazin-6-yl)-benzonitrile; 8-Methyl-6-(3-trifluoromethoxy-phenyl)-4H-benzo[1,4]oxazin-3-one; 6-Benzo[b]thiophen-5-yl-8-methyl-4H-benzo[1,4]oxazin-3-one; 6-(1H-Indazol-5-yl)-8-methyl-4H-benzo[1,4]oxazin-3-one; 6-(1H-Indol-6-yl)-8-methyl-4H-benzo[1,4]oxazin-3-one; 6-Benzyl-4H-benzo[1,4]oxazin-3-one; 6-Phenyl-4H-benzo[1,4]thiazin-3-one; 8-Chloro-6-m-tolyl-4H-benzo[1,4]oxazin-3-one; 6-[10,11]-dihydro-dibenzo[a,d]cyclohepten-5-ylidenemethyl]-4H-benzo[1,4]oxazin-3-one; 6-[10,11-dihydro-dibenzo[a,d]cyclohepten-5-ylidenemethyl]-8-fluoro-4H-benzo[1,4]oxazin-3-one; 6-[10,11]-dihydro-dibenzo[a,d]cyclohepten-5-ylidenemethyl]-8-methyl-4H-benzo[1,4]oxazin-3-one; 6-[10,11-dihydro-dibenzo[a,d]cyclohepten-4-benzyloxy-5-ylidenemethyl]-4H-benzo[1,4]oxazin-3-one (Z isomer); 6-[10,11-dihydro-dibenzo[a,d]cyclohepten-4-benzyloxy-5-ylidenemethyl]-4H-benzo[1,4]oxazin-3-one (E isomer); 6-[10,11-dihydro-dibenzo[a,d]cyclohepten-4-benzyloxy-5-ylidenemethyl]-8-methyl-4H-benzo[1,4]oxazin-3-one (Z isomer); 6-[10,11-dihydro-dibenzo[a,d]cyclohepten-4-benzyloxy-5-ylidenemethyl]-8-methyl-4H-benzo[1,4]oxazin-3-one (E isomer); 6-[(10,11-dihydro-dibenzo[a,d]cyclohepten-5-ylidene)ethyl]-8-methyl-4H-benzo[1,4]oxazin-3-one; 6-[10,11-dihydro-dibenzo[a,d]cyclohepten-4-hydroxy-5-ylidenemethyl]-8-methyl-4H-benzo[1,4]oxazin-3-one (E isomer); 6-[10,11-dihydro-dibenzo[a,d]cyclohepten-5-ylidenemethyl]-4H-benzo[1,4]thioxazin-3-one; 6-[10,11-dihydro-dibenzo[a,d]cyclohepten-5-ylidenemethyl]-4,4-dimethyl-benzo[1,4]oxazin-3-one; 6-((9H-thioxanthen-9-ylidene)methyl)-8-methyl-2H-benzo[b][1,4]oxazin-3(4H)-one; 6-[4-fluoro-8-methoxy-6H-dibenzo[b,e]oxepin-11-ylidenemethyl]-8-methyl-4H-benzo[1,4]oxazin-3-one; 7-m-tolylquinoxalin-2(1H)-one; 6-(2-Phenyl-thiazol-4-yl)-4H-benzo[1,4]oxazin-3-one; 8-Methyl-6-(2-pyridin-3-yl-thiazol-4-yl)-4H-benzo[1,4]oxazin-3-one; 6-[2-(3-Fluoro-phenyl)-thiazol-4-yl]-4H-benzo[1,4]oxazin-3-one; 6-[2-(3-Amino-phenyl)-thiazol-4-yl]-4H-benzo[1,4]oxazin-3-one; 5-Methyl-6-(2-pyridin-3-yl-thiazol-4-yl)-4H-benzo[1,4]oxazin-3-one; 5-Methyl-6-(2-phenyl-thiazol-4-yl)-4H-benzo[1,4]oxazin-3-one; 6-[2-(4-Fluoro-phenyl)-thiazol-4-yl]-5-methyl-4H-benzo[1,4]oxazin-3-one; 6-(2-Ethyl-thiazol-4-yl)-5-methyl-4H-benzo[1,4]oxazin-3-one; 6-(2-Benzo[1,3]dioxol-5-yl-thiazol-4-yl)-4H-benzo[1,4]oxazin-3-one; 6-[2-(2,3-Dihydro-benzo[1,4]dioxin-6-yl)-thiazol-4-yl]-4H-benzo[1,4]oxazin-3-one; 6-(2′-Methyl-[2,4′]bithiazolyl-4-yl)-4H-benzo[1,4]oxazin-3-one; 6-[2-(6-Methyl-pyridin-3-yl)-thiazol-4-yl]-4H-benzo[1,4]oxazin-3-one; 6-(2-Thiophen-3-yl-thiazol-4-yl)-4H-benzo[1,4]oxazin-3-one; [4-(3-Oxo-3,4-dihydro-2H-benzo[1,4]oxazin-6-yl)-thiazol-2-yl]-acetonitrile; 6-[2-(2-Trifluoromethyl-phenyl)-thiazol-4-yl]-4H-benzo[1,4]oxazin-3-one; 8-Methyl-6-(2-phenyl-thiazol-4-yl)-4H-benzo[1,4]oxazin-3-one; 6-(2-Ethyl-thiazol-4-yl)-8-methyl-4H-benzo[1,4]oxazin-3-one; 6-[2-(3-Hydroxy-phenyl)-thiazol-4-yl]-8-methyl-4H-benzo[1,4]oxazin-3-one; 6-(4-Phenyl-thiazol-2-yl)-4H-benzo[1,4]oxazin-3-one; 6-(4-Pyridin-3-yl-thiazol-2-yl)-4H-benzo[1,4]oxazin-3-one; 6-[2-(3-Amino-phenyl)-thiazol-4-yl]-8-methyl-4H-benzo[1,4]oxazin-3-one; 6-[2-(2,3-Dihydro-benzofuran-5-yl)-thiazol-4-yl]-8-methyl-4H-benzo[1,4]oxazin-3-one; 6-[2-(3-Amino-phenyl)-thiazol-4-yl]-5-methyl-4H-benzo[1,4]oxazin-3-one; 5-Methyl-6-[2-(2-trifluoromethyl-phenyl)-thiazol-4-yl]-4H-benzo[1,4]oxazin-3-one; 5-Methyl-6-[2-(6-methyl-pyridin-3-yl)-thiazol-4-yl]-4H-benzo-[1,4]oxazin-3-one; 5-Methyl-6-(2-thiophen-3-yl-thiazol-4-yl)-4H-benzo[1,4]oxazin-3-one; 6-[2-(2,3-Dihydro-benzo[1,4]dioxin-6-yl)-thiazol-4-yl]-5-methyl-4H-benzo[1,4]-oxazin-3-one; 8-Methyl-6-(4-pyridin-3-yl-thiazol-2-yl)-4H-benzo[1,4]oxazin-3-one; 8-Methyl-6-(4-thiophen-3-yl-thiazol-2-yl)-4H-benzo[1,4]oxazin-3-one; 8-Methyl-6-(2-thio-phen-3-yl-thiazol-4-yl)-4H-benzo[1,4]oxazin-3-one; 4-Acetyl-6-(2-thiophen-3-yl-thiazol-4-yl)-4H-benzo[1,4]oxazin-3-one; 8-Fluoro-6-(2-thiophen-3-yl-thiazol-4-yl)-4H-benzo-[1,4]oxazin-3-one; -[2-(2-Amino-pyridin-3-yl)-thiazol-4-yl]-4H-benzo[1,4]oxazin-3-one; 8-Fluoro-6-(2-pyridin-3-yl-thiazol-4-yl)-4H-benzo[1,4]oxazin-3-one; 8-Chloro-6-(2-pyridin-3-yl-thiazol-4-yl)-4H-benzo[1,4]oxazin-3-one; 6-[4-(3-Methoxy-phenyl)-thiazol-2-yl]-4H-benzo[1,4]oxazin-3-one; 6-[4-(6-Methyl-pyridin-3-yl)-thiazol-2-yl]-4H-benzo-[1,4]oxazin-3-one; 6-[2-(Methyl-phenyl-amino)-thiazol-4-yl]-4H-benzo[1,4]oxazin-3-one; 6-(2-Ethyl-thiazol-4-yl)-4H-benzo[1,4]oxazin-3-one; 6-(2,5-Dimethyl-thiazol-4-yl)-4H-benzo[1,4]oxazin-3-one; 6-(2-Pyridin-2-yl-thiazol-4-yl)-4H-benzo[1,4]oxazin-3-one; 6-(2-m-Tolyl-thiazol-4-yl)-4H-benzo[1,4]oxazin-3-one; 6-[2-(4-Hydroxy-phenyl)-thiazol-4-yl]-4H-benzo[1,4]oxazin-3-one; 6-(2-p-Tolyl-thiazol-4-yl)-4H-benzo[1,4]oxazin-3-one; 6-(2-Thiophen-2-yl-thiazol-4-yl)-4H-benzo[1,4]oxazin-3-one; 6-[2-(2-Hydroxy-phenyl)-thiazol-4-yl]-4H-benzo[1,4]oxazin-3-one; 6-[2-(3-Hydroxy-phenyl)-thiazol-4-yl]-4H-benzo[1,4]oxazin-3-one; 6-[2-(2-Fluoro-phenyl)-thiazol-4-yl]-4H-benzo[1,4]oxazin-3-one; 6-[2-(4-Fluoro-phenyl)-thiazol-4-yl]-4H-benzo[1,4]oxazin-3-one; 6-[2-(3-Chloro-phenyl)-thiazol-4-yl]-4H-benzo[1,4]oxazin-3-one; 6-[2-(4-Chloro-phenyl)-thiazol-4-yl]-4H-benzo[1,4]oxazin-3-one; 6-[2-(3-Trifluoromethyl-phenyl)-thiazol-4-yl]-4H-benzo[1,4]oxazin-3-one; 6-[2-(2,3-Dihydro-benzofuran-5-yl)-thiazol-4-yl]-4H-benzo[1,4]oxazin-3-one; [4-(3-Oxo-3,4-dihydro-2H-benzo[1,4]oxazin-6-yl)-thiazol-2-ylmethyl]-carbamic acid benzyl ester; 6-[2-(4-Fluoro-phenyl)-thiazol-4-yl]-8-methyl-4H-benzo[1,4]oxazin-3-one; 6-[2-(6-Methoxy-pyridin-3-yl)-thiazol-4-yl]-4H-benzo[1,4]oxazin-3-one; 6-[2-(2,3-Dihydro-benzofuran-5-yl)-thiazol-4-yl]-5-methyl-4H-benzo[1,4]oxazin-3-one; 5-Methyl-6-(2′-methyl-[2,4′]bithiazolyl-4-yl)-4H-benzo[1,4]oxazin-3-one; 6-[2-(2-Fluoro-phenyl)-thiazol-4-yl]-5-methyl-4H-benzo[1,4]oxazin-3-one; 6-[2-(4-Fluoro-phenyl)-thiazol-4-yl]-5-methyl-4H-benzo[1,4]oxazin-3-one; 5-Methyl-6-[2-(4-trifluoromethyl-pyridin-3-yl)-thiazol-4-yl]-4H-benzo[1,4]oxazin-3-one; 6-(4-Thiophen-3-yl-thiazol-2-yl)-4H-benzo[1,4]oxazin-3-one; 6-[2-(2-Chloro-pyridin-3-yl)-thiazol-4-yl]-4H-benzo[1,4]oxazin-3-one; 6-[4-(4-Fluoro-phenyl)-thiazol-2-yl]-4H-benzo[1,4]oxazin-3-one; 8-Methyl-6-[2-(6-methyl-pyridin-3-yl)-thiazol-4-yl]-4H-benzo[1,4]oxazin-3-one; 6-[4-(4-Chloro-phenyl)-thiazol-2-yl]-4H-benzo[1,4]oxazin-3-one; 6-[4-(4-Difluoromethoxy-phenyl)-thiazol-2-yl]-4H-benzo[1,4]oxazin-3-one; 6-(4-Benzo[1,3]dioxol-5-yl-thiazol-2-yl)-4H-benzo[1,4]oxazin-3-one; 6-[4-(2-Trifluoromethyl-phenyl)-thiazol-2-yl]-4H-benzo[1,4]oxazin-3-one; 6-[2-(6-Amino-pyridin-3-yl)-thiazol-4-yl]-8-methyl-4H-benzo[1,4]oxazin-3-one; 6-(8H-Indeno[1,2-d]thiazol-2-yl)-4H-benzo[1,4]oxazin-3-one; 6-[2-(5-Methyl-pyridin-3-yl)-thiazol-4-yl]-4H-benzo[1,4]oxazin-3-one; 6-[4-(4-Methoxy-phenyl)-thiazol-2-yl]-4H-benzo[1,4]oxazin-3-one; 6-[4-(3-Bromo-phenyl)-thiazol-2-yl]-4H-benzo[1,4]oxazin-3-one; 5-[2-(3-Oxo-3,4-dihydro-2H-benzo[1,4]oxazin-6-yl)-thiazol-4-yl]-nicotinonitrile; 6-[2-(3-Dimethylamino-phenyl)-thiazol-4-yl]-8-methyl-4H-benzo[1,4]oxazin-3-one; 6-(5-Acetyl-4-methyl-thiazol-2-yl)-8-methyl-4H-benzo[1,4]oxazin-3-one; 6-(5,6-Dihydro-4H-cyclopentathiazol-2-yl)-8-methyl-4H-benzo[1,4]oxazin-3-one; 6-(4,5,6,7-Tetrahydro-benzothiazol-2-yl)-4H-benzo[1,4]oxazin-3-one; 6-(4-Trifluoromethyl-thiazol-2-yl)-4H-benzo[1,4]oxazin-3-one; 6-[2-(2-Amino-pyridin-3-yl)-thiazol-4-yl]-8-fluoro-4H-benzo[1,4]oxazin-3-one; 6-(4-Hydroxymethyl-thiazol-2-yl)-8-methyl-4H-benzo[1,4]oxazin-3-one; 6-(4,5-Dihydro-2-oxa-6-thia-1,3,8-triaza-as-indacen-7-yl)-4H-benzo[1,4]oxazin-3-one; 6-[2-(6-Amino-pyridin-2-yl)-thiazol-4-yl]-4H-benzo[1,4]oxazin-3-one; 6-[2-(1H-Indol-4-yl)-thiazol-4-yl]-8-methyl-4H-benzo[1,4]oxazin-3-one; 6-[2-(1H-Indazol-5-yl)-thiazol-4-yl]-4H-benzo[1,4]oxazin-3-one; 6-[2-(1H-Indazol-5-yl)-thiazol-4-yl]-8-methyl-4H-benzo[1,4]oxazin-3-one; 6-(2-Pyrazin-2-yl-thiazol-4-yl)-4H-benzo[1,4]oxazin-3-one; 6-[2-(2-Amino-pyridin-3-yl)-thiazol-4-yl]-5-methyl-4H-benzo[1,4]oxazin-3-one; 6-[2-(2-Amino-pyridin-3-yl)-thiazol-4-yl]-8-methyl-4H-benzo[1,4]oxazin-3-one; 5,8-Dimethyl-6-(2-pyridin-3-yl-thiazol-4-yl)-4H-benzo[1,4]oxazin-3-one; 6-[2-(5-Amino-pyridin-3-yl)-thiazol-4-yl]-4H-benzo[1,4]oxazin-3-one; 8-Fluoro-6-(4-pyridin-3-yl-thiazol-2-yl)-4H-benzo[1,4]oxazin-3-one; 7-(4-(thiophen-3-yl)thiazol-2-yl)quinoxalin-2(1H)-one; 3,4-dihydro-7-(4-(thiophen-3-yl)thiazol-2-yl)quinoxalin-2(1H)-one; 6-(2-(5-amino-2-methylphenyl)thiazol-4-yl)-2H-benzo[b][1,4]oxazin-3(4H)-one; 6-(2-(3-amino-4-fluorophenyl)thiazol-4-yl)-2H-benzo[b][1,4]oxazin-3(4H)-one; 6-(2-(2,6-dichloro-3-nitrophenyl)thiazol-4-yl)-2H-benzo[b][1,4]oxazin-3(4H)-one; 6-(2-(3-amino-4-hydroxyphenyl)thiazol-4-yl)-2H-benzo[b][1,4]oxazin-3(4H)-one; 6-(2-(3-amino-4-chlorophenyl)thiazol-4-yl)-2H-benzo[b][1,4]oxazin-3(4H)-one; 6-(2-(3-amino-4-methylphenyl)thiazol-4-yl)-8-methyl-2H-benzo[b][1,4]oxazin-3(4H)-one; 6-(2-(3-amino-2-methylphenyl)thiazol-4-yl)-2H-benzo[b][1,4]oxazin-3(4H)-one; 6-(2-(3-amino-4-methylphenyl)thiazol-4-yl)-5-methyl-2H-benzo[b][1,4]oxazin-3(4H)-one; 6-(2-(3-(ethylamino)phenyl)thiazol-4-yl)-8-methyl-2H-benzo[b][1,4]oxazin-3(4H)-one; N-(3-(4-(3,4-dihydro-8-methyl-3-oxo-2H-benzo[b]-[1,4]oxazin-6-yl)thiazol-2-yl)phenyl)acetamide; N-(3-(4-(3,4-dihydro-8-methyl-3-oxo-2H-benzo[b]-[1,4]oxazin-6-yl)thiazol-2-yl)phenyl)sulfonamide; 6-(2-(3-(benzylamino)phenyl)thiazol-4-yl)-8-methyl-2H-benzo[b][1,4]oxazin-3(4H)-one; 6-(2-(3-amino-4-fluorophenyl)thiazol-4-yl)-5-methyl-2H-benzo[b][1,4]oxazin-3(4H)-one; 6-(2-(3-amino-4-fluorophenyl)thiazol-4-yl)-8-methyl-2H-benzo[b][1,4]oxazin-3(4H)-one; 6-(2-(3-amino-4-methylphenyl)thiazol-4-yl)-5,8-dimethyl-2H-benzo[b][1,4]oxazin-3(4H)-one; 6-(2-(3-amino-5-fluorophenyl)thiazol-4-yl)-2H-benzo[b][1,4]oxazin-3(4H)-one; 6-(2-(3-amino-5-fluorophenyl)thiazol-4-yl)-8-methyl-2H-benzo[b][1,4]oxazin-3(4H)-one; 6-(2-(3-amino-5-fluorophenyl)thiazol-4-yl)-8-fluoro-2H-benzo[b][1,4]oxazin-3(4H)-one; 6-(2-(3-amino-5-fluorophenyl)thiazol-4-yl)-8-chloro-2H-benzo[b][1,4]oxazin-3(4H)-one; 6-(2-(3-amino-5-fluorophenyl)thiazol-4-yl)-5-methyl-2H-benzo[b][1,4]oxazin-3(4H)-one; 6-(2-(3-amino-5-fluorophenyl)thiazol-4-yl)-5,8-dimethyl-2H-benzo[b][1,4]oxazin-3(4H)-one; 6-[2-(4-Hydroxy-2-methyl-phenyl)-vinyl]-4H-benzo[1,4]oxazin-3-one; 6-[2-(4-Hydroxy-3-methyl-phenyl)-vinyl]-4H-benzo[1,4]oxazin-3-one; 6-[2-(3-Fluoro-4-hydroxy-phenyl)-vinyl]-4H-benzo[1,4]oxazin-3-one; 6-[2-(3-Hydroxy-phenyl)-ethyl]-4H-benzo[1,4]oxazin-3-one; 6-[2-(4-Hydroxy-2-methyl-phenyl)-ethyl]-4H-benzo[1,4]oxazin-3-one; 6-[2-(4-Hydroxy-3-methyl-phenyl)-vinyl]-8-methyl-4H-benzo[1,4]oxazin-3-one; 6-(3,4-Dihydro-1H-isoquinolin-2-yl)-4H-benzo[1,4]oxazin-3-one; 6-(3,4-Dihydro-1H-isoquinolin-2-yl)-8-methyl-4H-benzo[1,4]oxazin-3-one; 6-(4,7-Dihydro-5H-thieno[2,3-c]pyridin-6-yl)-8-methyl-4H-benzo[1,4]oxazin-3-one; 6-(3,4-Dihydro-1H-isoquinolin-2-yl)-8-fluoro-4H-benzo[1,4]oxazin-3-one; 8-Chloro-6-(3,4-dihydro-1H-isoquinolin-2-yl)-4H-benzo[1,4]oxazin-3-one; 6-(dibenzylamino)-2H-benzo[b]-[1,4]oxazin-3(4H)-one; 3-Oxo-6-(2-pyridin-3-yl-thiazol-4-yl)-3,4-dihydro-2H-benzo[1,4]oxazine-8-carbonitrile; 6-(2-Pyridin-3-yl-oxazol-5-yl)-4H-benzo[1,4]oxazin-3-one; 6-(2-Phenyl-oxazol-4-yl)-4H-benzo[1,4]oxazin-3-one; 4-Methanesulfonyl-6-(2-phenyl-thiazol-4-yl)-4H-benzo[1,4]oxazin-3-one; 4-Acetyl-6-[4-(3-bromo-phenyl)-thiazol-2-yl]-4H-benzo[1,4]oxazin-3-one; 8-Methyl-6-[3-(2,2,2-trifluoro-1-hydroxy-ethyl)-phenyl]-4H-benzo[1,4]oxazin-3-one; 6-[3-Chloro-5-(1-hydroxy-ethyl)-phenyl]-8-methyl-4H-benzo[1,4]oxazin-3-one; 8-Methyl-6-(3-pyrazol-1-ylmethyl-phenyl)-4H-benzo[1,4]oxazin-3-one; 6-[3-(3-Trifluoromethyl-phenyl)-acryloyl]-4H-benzo[1,4]oxazin-3-one; 4-[3-(3-Oxo-3,4-dihydro-2H-benzo[1,4]oxazin-6-yl)-5-phenyl-4,5-dihydro-pyrazol-1-yl]-benzonitrile; 6-(1-Phenyl-1H-pyrazol-3-yl)-4H-benzo[1,4]oxazin-3-one; 6-(1,5-Diphenyl-1H-pyrazol-3-yl)-4H-benzo[1,4]oxazin-3-one; 6-(2-Phenyl-oxazol-4-yl)-4H-benzo[1,4]oxazin-3-one; and 6-(3-phenyl-1,2,4-oxadiazol-5-yl)-2H-benzo[b][1,4]oxazin-3(4H)-one.

6. A pharmaceutical composition comprising a therapeutically effective amount of a compound of claim 1 in combination with a pharmaceutically acceptable excipient.

7. A method for treating a disease in an animal in which modulation of steroid nuclear hormone receptor activity can prevent, inhibit or ameliorate the pathology and/or symptomology of the disease, which method comprises administering to the animal a therapeutically effective amount of a compound of claim 1.

8. The use of a compound of claim 1 in the manufacture of a medicament for treating a disease in an animal in which aberrant steroid nuclear hormone receptor activity contributes to the pathology and/or symptomology of the disease.
